(19) United States
(12) Patent Application Publication
(10) Pub. No.: US 2023/0310337 A1
(43) Pub. Date: Oct. 5, 2023

(54) DEVICES AND METHODS FOR THE TREATMENT OF SKIN DEPIGMENTATION

(71) Applicant: TEVIDO BIODEVICES, INC., Austin, TX (US)

(72) Inventors: Lindsay SULZER, Austin, TX (US); Derek HERNANDEZ, Austin, TX (US)

(21) Appl. No.: 18/003,152

(22) PCT Filed: Apr. 23, 2021
(86) PCT No.: PCT/US2021/028904

§ 371 Date: Dec. 22, 2022

Related U.S. Application Data
(60) Provisional application No. 63/043,652, filed on Jun. 24, 2020.

(51) Int. Cl. A61K 9/70; A61L 24/10

(52) U.S. Cl. CPC A61K 9/7038; A61L 24/106; A61L 15/32

(57) ABSTRACT

A cellularized patch for treating a scar or skin condition of a subject The cellularized patch and methods of use thereof are advantageous in skin graft procedures performed, for example, to treat a subject having a skin condition comprising skin hypopigmentation or depigmentation, such as vitiligo. In some cases, human melanocytes are cultured ex vivo and seeded onto a transfer patch before being applied to a subject.

### GOVERNMENT SUPPORT

This invention was made with Government support under Grant Number IIP-1431019 awarded by the National Science Foundation. The Government has certain rights in this invention.

### FIELD OF THE TECHNOLOGY

The present disclosure generally relates to cellularized patch devices and methods of use thereof useful in treating a subject having a scar or skin condition comprising skin hypopigmentation or depigmentation, such as vitiligo.

### BACKGROUND

Permanent depigmentation of the skin is prevalent and is caused by a wide range of local and systemic conditions. Melanocytes produce melanin, which is primarily responsible for skin pigmentation in an individual. Depigmentation of the skin can be the result of loss of melanocytes.

For individuals with a complete loss of pigmentation in a portion of skin, such as those suffering from vitiligo, there are limited options to restore lost pigmentation in the skin.

Current devices and methods for restoring pigmentation in skin perform poorly when grafting is needed on curved skin surfaces and hard-to-treat areas, such as around arms, knees, or elbows of a subject. For example, current devices and methods often result in low efficiency of cell transfer to a treatment site of a subject and can fail to achieve homogeneous graft coverage and growth, which often leads to mismatch or undesired heterogeneity of skin pigmentation. Thus, there is a long-felt and unmet need for an effective treatment of depigmentation of the skin, especially in portions of the skin having geometries that make grafting difficult.

### SUMMARY

The present disclosure generally relates to devices and methods useful in treating a skin condition in a patient, or a symptom of a skin condition in a patient, that affects pigmentation of the skin of the patient. The devices and methods of the present disclosure are particularly useful for the treatment of hypopigmentation of the skin, depigmentation of the skin, and skin scarring, for example, as a result of wound healing or disease. In some cases, the depigmentation is caused by a disease, such as vitiligo.

Two major challenges for the repigmentation of a portion of a subject's skin are (1) maintaining a desired spatial distribution of cells (e.g., pigment-producing cells, such as melanocytes) during and after application of the cells to the portion of the subject's body and (2) ensuring that cells (e.g., pigment-producing cells) applied to the subject's body incorporate into the subject's body efficiently. Some existing skin grafting strategies involve transplanting autologous skin cells (e.g., cells that express melanin, such as melanocytes)) into the depigmented portion (e.g., target area) of the skin; however, many of these strategies result in poor survival of transferred cells (e.g., less than 60%). Non-cultured autologous melanocyte transplanting strategies (e.g., wherein an autologous melanocyte is isolated and applied without proliferative expansion in culture) are also only expected to result in repigmentation of about 50% of a given vitiligo lesion area 6 months after transplant. That is, in many cases existing systems and methods for repigmentation of skin result in uneven or undesired distribution of pigmentation at a target area of a subject after treatment. In some cases, this results from a lack of control over the distribution of pigment-producing cells during and after transfer to the target area of the subject and can necessitate additional interventions (e.g., additional applications of pigment-producing cells) to obtain a desired distribution of pigmentation at the target area (e.g., a more even distribution of pigmentation at the target area). Additionally, current skin grafting strategies often result in low pigmentation efficiency and/or incomplete treatment of areas of a subject's body where the skin is rounded or curved, such as the surfaces of knees, elbows, and arms.

The devices and methods disclosed herein overcome obstacles associated with treating skin depigmentation, such as limited quantities of cellular reagents, challenges in achieving a desired distribution of pigment-producing cells at a target area, and skin surface geometries (e.g., curved or rounded surfaces) that make it difficult to apply cellular suspensions by providing methods for purification, expansion, and incorporation of specific cell populations (e.g., primary human melanocytes or melanocytes in combination with other epidermal cells such as keratinocytes) into transplant systems (e.g., a patch device disclosed herein) capable of efficiently transferring purified and expanded melanocytes (or, optionally, a combination of expanded melanocytes and keratinocytes) to a wide range of target skin areas of a subject, including those that prove difficult for current technologies, including knees, elbows, and arms.

Provided herein is a cellularized patch device for treating subjects having skin hypopigmentation or depigmentation, the cellularized patch device comprising: a gel substrate having a first surface and a second surface; a cellular component disposed within the gel substrate, the cellular component comprising a population of cells, wherein at least 80 percent of the population of cells is disposed within a distance from the second surface of no more than 50 percent of the height of the gel substrate. In some embodiments, at least 80 percent of the population of cells is disposed within a distance from the second surface of no more than 30 percent of the height of the gel substrate. In some embodiments, at least 80 percent of the population of cells is disposed within a distance from the second surface of no more than 10 percent of the height of the gel substrate.

Provided herein is a cellularized patch device, comprising: a gel substrate having a first surface and a second surface, and comprising from 10 mg/mL to 15 mg/mL fibrin and 2 U/mL thrombin; a cellular component comprising a population of cells, the population of cells comprising a plurality of human melanocytes and wherein the population of cells is disposed within the gel substrate; and an adhesive.

In some embodiments, the population of cells is at least 90% primary human melanocytes. In some embodiments, a concentration of human melanocytes in the device is from 50,000 cells/cm² to 350,000 cells/cm². In some embodiments, the human melanocytes are applied to the first surface of the gel substrate during gel substrate formation. In some embodiments, the gel substrate comprises 10 mg/mL fibrin.

In some embodiments, the cellularized patch device further comprises an adhesive. In some embodiments, the adhesive is applied to the second surface of the gel substrate during gel substrate formation. In some embodiments, the adhesive comprises thrombin. In some embodiments, a concentration of the thrombin in the adhesive is from 1 U/mL to 10 U/mL. In some embodiments the concentration of the thrombin in the adhesive is 2 U/mL. In some embodiments, the adhesive comprises fibrin. In some embodiments, a concentration of the fibrin in the adhesive is 5 mg/mL. In some embodiments, the adhesive further comprises hyaluronic acid. In some embodiments, a concentration of the hyaluronic acid in the adhesive is 1 mg/mL.

In some embodiments, the cellularized patch device further comprises a backing component. In some embodiments, the backing component is coupled to the first surface of the gel substrate. In some embodiments, the backing component is flexible. In some embodiments, the backing component comprises a fibrin cap. In some embodiments, the fibrin cap comprises fibrin. In some embodiments, the fibrin cap comprises 15 mg/mL of fibrin. In some embodiments, the fibrin cap comprises at least 3 mg/mL of fibrin, at least 5 mg/mL of fibrin, at least 6 mg/mL of fibrin, at least 7 mg/mL of fibrin, at least 8 mg/mL of fibrin, at least 9 mg/mL of fibrin, at least 10 mg/mL of fibrin, at least 11 mg/mL of fibrin, at least 12 mg/mL of fibrin, at least 13 mg/mL of fibrin, at least 14 mg/mL of fibrin, at least 15 mg/mL of fibrin, at least 16 mg/mL of fibrin, at least 17 mg/mL of fibrin, at least 18 mg/mL of fibrin, at least 19 mg/mL of fibrin, at least 20 mg/mL of fibrin, at least 25 mg/mL of fibrin, at least 30 mg/mL of fibrin, at least 17.5 mg/mL of fibrin, at least 22.5 mg/mL of fibrin, at least 17.5 mg/mL of fibrin, at least 35 mg/mL of fibrin, at least 3 mg/mL to at most 50 mg/mL of fibrin, at least 5 mg/mL to at most 40 mg/mL of fibrin, at least 7 mg/mL to at most 35 mg/mL of fibrin, at least 9 mg/mL to at least 30 mg/mL of fibrin, at least 10 mg/mL to at least 50 mg/mL of fibrin, at least 10 mg/mL to at least 40 mg/mL of fibrin, at least 10 mg/mL to at least 30 mg/mL of fibrin, at least 10 mg/mL to at least 20 mg/mL of fibrin, at least 10 mg/mL to at least 100 mg/mL of fibrin, at least 10 mg/mL to at least 75 mg/mL of fibrin, at least 10 mg/mL to at least 60 mg/mL of fibrin, at least 10 mg/mL to at least 25 mg/mL of fibrin, at least 15 mg/mL to at least 50 mg/mL of fibrin, at least 15 mg/mL to at least 100 mg/mL of fibrin, at least 15 mg/mL to at least 45 mg/mL of fibrin, at least 15 mg/mL to at least 200 mg/mL of fibrin, at least 15 mg/mL to at least 75 mg/mL of fibrin, at least 1 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, about 3 mg/mL of fibrin, about 5 mg/mL of fibrin, about 6 mg/mL of fibrin, about 7 mg/mL of fibrin, about 8 mg/mL of fibrin, about 9 mg/mL of fibrin, about 10 mg/mL of fibrin, about 11 mg/mL of fibrin, about 12 mg/mL of fibrin, about 13 mg/mL of fibrin, about 14 mg/mL of fibrin, about 15 mg/mL of fibrin, about 16 mg/mL of fibrin, about 17 mg/mL of fibrin, about 18 mg/mL of fibrin, about 19 mg/mL of fibrin, about 20 mg/mL of fibrin, about 25 mg/mL of fibrin, about 30 mg/mL of fibrin, about 17.5 mg/mL of fibrin, about 22.5 mg/mL of fibrin, and/or about 17.5 mg/mL of fibrin, about 35 mg/mL of fibrin. In some embodiments, the fibrin cap comprises thrombin. In some embodiments, the fibrin cap comprises from 1 U/mL to 10 U/mL thrombin. In some embodiments, the fibrin cap comprises 2 U/mL of thrombin. In some embodiments, the fibrin cap comprises hyaluronic acid. In some embodiments, the fibrin cap comprises from 0.5 mg/mL to 1.5 mg/mL hyaluronic acid. In some embodiments, the backing component comprises a silicone dressing.

Provided herein is a method of fabricating a cellularized patch device, comprising: isolating a plurality of primary human melanocytes; mixing isolated primary human melanocytes with fibrinogen and thrombin to obtain a mixture capable of forming a gel substrate, wherein concentration of the fibrinogen in the mixture is from 10 mg/mL to 15 mg/mL and concentration of the thrombin in the mixture is 2 U/mL; forming the gel substrate having a first surface and a second surface; applying a 10 μL to 50 μL droplet of an adhesive to the second surface. In some embodiments, the method further comprises incubating the gel substrate for 20-30 minutes at room temperature after the mixing step. In some embodiments, wherein the gel substrate is placed in a mold during the incubating step. In some embodiments, the method further comprises applying a droplet of an adhesive to the second surface. In some embodiments, the method further comprises applying a 10 μL to 50 μL droplet of an adhesive to the second surface. In some embodiments, the droplet has a volume of 50 μL. In some embodiments, the droplet has a volume of 10 μL. In some embodiments, the adhesive comprises thrombin. In some embodiments, the concentration of the thrombin in the adhesive is from 1 U/mL to 10 U/mL. In some embodiments, the concentration of the thrombin in the adhesive is 2 U/mL. In some embodiments, the adhesive comprises fibrin. In some embodiments, the concentration of the fibrin in the adhesive is 5 mg/mL. In some embodiments, the adhesive comprises hyaluronic acid. In some embodiments, the concentration of the hyaluronic acid in the adhesive is 1 mg/mL

In some embodiments, the method further comprising providing a backing component. In some embodiments, the method further comprises coupling the backing component to the first surface of the gel substrate. In some embodiments, the backing component is flexible. In some embodiments, the backing component comprises a fibrin cap. In some embodiments, the fibrin cap comprises fibrin. In some embodiments, the fibrin cap comprises at least 15 mg/mL of fibrin. In some embodiments, the fibrin cap comprises at least 3 mg/mL of fibrin, at least 5 mg/mL of fibrin, at least 6 mg/mL of fibrin, at least 7 mg/mL of fibrin, at least 8 mg/mL of fibrin, at least 9 mg/mL of fibrin, at least 10 mg/mL of fibrin, at least 11 mg/mL of fibrin, at least 12 mg/mL of fibrin, at least 13 mg/mL of fibrin, at least 14 mg/mL of fibrin, at least 15 mg/mL of fibrin, at least 16 mg/mL of fibrin, at least 17 mg/mL of fibrin, at least 18 mg/mL of fibrin, at least 19 mg/mL of fibrin, at least 20 mg/mL of fibrin, at least 25 mg/mL of fibrin, at least 30 mg/mL of fibrin, at least 17.5 mg/mL of fibrin, at least 22.5 mg/mL of fibrin, at least 17.5 mg/mL of fibrin, at least 35 mg/mL of fibrin, at least 3 mg/mL to at most 50 mg/mL of fibrin, at least 5 mg/mL to at most 40 mg/mL of fibrin, at least 7 mg/mL to at most 35 mg/mL of fibrin, at least 9 mg/mL to at least 30 mg/mL of fibrin, at least 10 mg/mL to at least 50 mg/mL of fibrin, at least 10 mg/mL to at least 40 mg/mL of fibrin, at least 10 mg/mL to at least 30 mg/mL of fibrin, at least 10 mg/mL to at least 20 mg/mL of fibrin, at least 10 mg/mL to at least 100 mg/mL of fibrin, at least 10 mg/mL to at least 75 mg/mL of fibrin, at least 10 mg/mL to at least 60 mg/mL of fibrin, at least 10 mg/mL to at least 25 mg/mL of fibrin, at least 15 mg/mL to at least 50 mg/mL of fibrin, at least 15 mg/mL to at least 100 mg/mL of fibrin, at least 15 mg/mL to at least 45 mg/mL of fibrin, at least 15 mg/mL to at least 200 mg/mL of fibrin, at least 15 mg/mL to at least 75 mg/mL of fibrin, at least 1 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, about 3 mg/mL of fibrin, about 5 mg/mL of fibrin, about 6 mg/mL of fibrin, about 7 mg/mL of fibrin, about 8 mg/mL of fibrin, about 9 mg/mL of fibrin, about 10 mg/mL of fibrin, about 11 mg/mL of fibrin, about 12 mg/mL of fibrin, about 13 mg/mL of fibrin, about 14 mg/mL of fibrin, about 15 mg/mL of fibrin, about 16 mg/mL of fibrin, about 17 mg/mL of fibrin, about 18 mg/mL of fibrin, about 19 mg/mL of fibrin, about 20 mg/mL of fibrin, about 25 mg/mL of fibrin, about 30 mg/mL of fibrin, about 17.5 mg/mL of fibrin, about 22.5 mg/mL of fibrin, and/or about 17.5 mg/mL of fibrin, about 35 mg/mL of fibrin. In some embodiments, the fibrin cap comprises thrombin. In some embodiments, the fibrin cap comprises from 1 U/mL to 10 U/mL thrombin. In some embodiments, the fibrin cap comprises 2 U/mL of thrombin. In some embodiments, the fibrin cap comprises hyaluronic acid. In some embodiments, the fibrin cap comprises from 0.5 mg/mL to 1.5 mg/mL hyaluronic acid. In some embodiments, the backing component comprises a silicone dressing. In some embodiments, isolating the plurality of primary human melanocytes comprises enzymatic digestion. In some embodiments, isolating the plurality of primary human melanocytes comprises dissecting an epidermis of a skin sample from the subject from a dermis of the skin sample. In some embodiments, the plurality of primary human melanocytes are isolated without a mechanical dissection step.

Provided herein is a method of treating a skin condition of a subject in need thereof, comprising: fabricating a patch device comprising: (i) a cellular component comprising a population of cells, the population of cells comprising a plurality of human melanocytes, (ii) a gel substrate having a first surface and a second surface, and comprising from 10 mg/mL to 15 mg/mL fibrin and 2 U/mL thrombin, and (iii) an adhesive applied to the second surface of the gel substrate; and applying the second surface to a target tissue in a treatment area of a subject. Provided herein is a method of treating a skin condition of a subject in need thereof, comprising: fabricating a patch device comprising: (i) a gel substrate having a first surface and a second surface, and comprising from 10 mg/mL to 15 mg/mL fibrin and 2 U/mL thrombin, and (ii) a cellular component disposed within the gel substrate, the cellular component comprising a population of cells, wherein at least 80 percent of the population of cells is disposed within a distance from the second surface of no more than 50 percent of the height of the gel substrate; and applying the second surface to a target tissue in a treatment area of a subject. In some embodiments, at least 80 percent of the population of cells is disposed within a distance from the second surface of no more than 30 percent of the height of the gel substrate. In some embodiments, wherein at least 80 percent of the population of cells is disposed within a distance from the second surface of no more than 10 percent of the height of the gel substrate. In some embodiments, a method further comprises debriding the treatment area before applying the second surface of the patch device to the target tissue. In some embodiments, a method further comprises applying a pressure to the patch device oriented normal to the target tissue while the second surface of the patch device is applied to the target. In some embodiments, the pressure is applied to the patch device for a time of less than 1 minute. In some embodiments, the pressure is applied to the patch device for a time of from 1 minute to 72 hours. In some embodiments, the pressure is applied to the patch device for a time of 24 hours to 48 hours. In some embodiments, the method further comprises repeating the applying step. In some embodiments, the applying step is repeated using a second patch device comprising (i) a cellular component comprising a plurality of human melanocytes. (ii) a gel substrate having a first surface and a second surface, and comprising from 10 mg/mL to 15 mg/mL fibrin and 2 U/mL thrombin, and (iii) an adhesive applied to the second surface of the gel substrate. In some embodiments, a backing component is coupled to the first surface of the gel substrate. In some embodiments, a method further comprises removing the backing component from the first surface of the patch device during the applying step. In some embodiments, a method further comprises removing the backing component from the first surface of the patch device after the applying step. In some embodiments, the subject has vitiligo. In some embodiments, the target area includes one or more scars. In some embodiments, the target area comprises a portion of a scarred area. In some embodiments, a method further comprises controlling the spatial distribution of cells within the patch device. In some embodiments, a method further comprises delivering pigmented cells to the target area of the subject's skin. In some embodiments, a method further comprises maintaining the spatial distribution of the pigmented cells within the patch device during application of the patch device to the target area. In some embodiments, a method further comprises controlling the spatial distribution of the pigmented cells during transfer of the pigmented cells from the patch device to the target area.

Provided herein is a method of transferring pigment-producing cells to a target area of a surface of skin of a subject comprising: delivering a patch device comprising the pigment-producing cells to the target area of the surface of the skin of the subject; applying perpendicular pressure to the patch device in contact with the target area of the skin of the subject; and transferring at least 80% of the pigment-producing cells from the patch device to the target area of the surface of the skin. In some embodiments applying the patch device is effective to repigment the target area of the surface of the skin to at least 90%, at least 95%, at least 97%, or at least 99%.

Provided herein is a method of maintaining spatial distribution of pigment-producing cells on a target area of a surface of skin of a subject, the method comprising: providing a patch device having a three-dimensional gel substrate comprising pigment-producing cells, the gel substrate having a spatial distribution of the cells of from 75,000 cells/cm² to 325,000 cells/cm² in an x-y plane of the gel substrate, the x-y plane is at most 500 micrometers thick; applying the patch device to the target area of the surface of the skin; and delivering the pigment-producing cells to the target area of the surface of the skin, the target area of the surface of the skin having a spatial distribution of the pigment-producing cells of 75,000 cells/cm² to 325,000 cells/cm². Provided herein is a method of maintaining spatial distribution of pigment-producing cells on a target area of a surface of skin of a subject, the method comprising: providing a patch device having a three-dimensional gel substrate comprising pigment-producing cells, the gel substrate having a spatial distribution of the cells of about 230,000 cells/cm² in an x-y plane of the gel substrate, the x-y plane is at most 500 micrometers thick; applying the patch device to the target area of the surface of the skin; and delivering the pigment-producing cells to the target area of the surface of the skin, the target area of the surface of the skin having a spatial distribution of the pigment-producing cells of 75,000 cells/cm² to 230,000 cells/cm². Provided herein is a method of maintaining spatial distribution of pigment-producing cells on a target area of a surface of skin of a subject, the method comprising: providing a patch device having a three-dimensional gel substrate comprising pigment-producing cells, the gel substrate having a spatial distribution of the cells of about from 200,000 cells/cm² to 350,000 cells/cm² in an x-y plane of the gel substrate, the x-y plane is at most 500 micrometers thick; applying the patch device to the target area of the surface of the skin; and delivering the pigment-producing cells to the target area of the surface of the skin, the target area of the surface of the skin having a spatial distribution of the pigment-producing cells of 75,000 cells/cm² to 350,000 cells/cm². Provided herein is a method of maintaining spatial distribution of pigment-producing cells on a target area of a surface of skin of a subject, the method comprising: providing a patch device having a three-dimensional gel substrate comprising pigment-producing cells, the gel substrate having a spatial distribution of the cells of about from 125,000 cells/cm² to 250,000 cells/cm² in an x-y plane of the gel substrate, the x-y plane is at most 500 micrometers thick; applying the patch device to the target area of the surface of the skin; and delivering the pigment-producing cells to the target area of the surface of the skin, the target area of the surface of the skin having a spatial distribution of the pigment-producing cells of 75,000 cells/cm² to 250,000 cells/cm². In some embodiments, the cells are melanocytes.

Provided herein is a method of treating a subject with a skin pigmentation comprising administering to a target area of a surface of skin of the subject a patch device having a gel substrate comprising pigmented cells, wherein administration of the patch device is effective to repigment the target area of the surface of the skin to at least 80% of a reference surface of the skin, as measured by reflectance spectroscopy. In some embodiments, a method of treating a subject with a skin pigmentation comprising administering to a target area of a surface of skin of the subject a patch device having a gel substrate comprising pigmented cells, wherein administration of the patch device is effective to repigment the target area of the surface of the skin, as determined by visual inspection. In some embodiments, the repigmentation of the target area is determined by measuring the melanin index of the target area. In some embodiments, the repigmentation of the reference surface is determined by measuring the melanin index of the reference surface. In some embodiments, administration of the patch device is effective to repigment the target area of the surface of the skin to at least 90%, at least 95%, at least 97%, or at least 99%.

Provided herein is a method of treating a subject with skin depigmentation comprising administering to a target area of skin of the subject a patch device having a gel substrate comprising pigment-producing cells, wherein administration of the patch device is effective to transfer the pigment-producing cells to the target surface of the skin more evenly than an alternative repigmentation treatment, as determined using reflectance spectroscopy. In some embodiments, the alternative repigmentation treatment method comprises administration of cells in a non-viscous suspension, in a viscous suspension, using a rigid stamp, using a bandage, or using a tape. In some embodiments, the patch device used in a method disclosed herein is a cellularized patch device disclosed herein. In some embodiments, the patch device used in a method disclosed herein is fabricated using the method disclosed herein. In some embodiments, the patch device further comprises a backing component. In some embodiments, the backing component is a silicone dressing.

In some embodiments, a method disclosed herein further comprising culturing at least a portion of the population of cells. In some embodiments, a method disclosed herein further comprising culturing at least a portion of the population of cells for at least 5 passages. In some embodiments, a method disclosed herein further comprising culturing at least a portion of the plurality of melanocytes. In some cases, a method disclosed herein further comprising culturing at least a portion of the population of melanocytes for at least 5 passages. In some embodiments, at least a portion of the population of cells has been cultured. In some embodiments, at least a portion of the population of cells has been cultured for at least 5 passages.

Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows a schematic of a patch device during application, in accordance with embodiments.

FIG. 1B shows a schematic of a patch device after application, in accordance with embodiments.

FIG. 1C shows a schematic of a patch device, in accordance with embodiments.

FIG. 2 shows bright phase images of cells in multiple planes of a patch device, in accordance with embodiments.

FIG. 3 shows immunostaining results of primary human melanocyte cultures, in accordance with embodiments (scale bar indicates 1 mm, arrows indicate cells staining positive for K-14, positive staining without arrows indicates cells staining positive for TRP-1, asterisks indicate nuclear staining (DAPI) of cells not staining positively for K-14 or TRP-1 (which cells may include immune cells, Merkel cells, Langerhans, and/or fibroblasts)).

FIG. 4A shows calcein staining of human melanocytes in a fibrin gel, in accordance with embodiments.

FIG. 4B shows calcein staining of the human melanocytes after transfer from a fibrin gel to a plastic surface, in accordance with embodiments.

FIGS. 5A and 5B show calcein staining of human melanocytes 4 days after transfer, in accordance with embodiments.

FIG. 5C shows calcein staining of human melanocytes before removal of patch device backing component 5 days after transfer to a plastic surface, in accordance with embodiments.

FIG. 5D shows calcein staining of the human melanocytes of FIG. 5C after removal of patch device backing component 5 days after transfer to a plastic surface, in accordance with embodiments.

FIGS. 6A and 6B show digital camera images of pigmentation of human melanocytes 7 days after transfer to a plastic surface, in accordance with embodiments.

FIGS. 6C and 6D show digital camera images of pigmentation of the human melanocytes in FIGS. 6A and 6B, respectively, 10 days after transfer.

FIG. 7 shows steps of a method for fabrication and use of a patch device, in accordance with embodiments.

FIG. 8A and FIG. 8B show a method for isolating human melanocytes using a shave biopsy instrument, in accordance with embodiments.

FIG. 9A shows pigmentation of primary human melanocytes cultured for 1, 6, 13, and 20 days.

FIG. 9B shows pigmentation of the primary human melanocytes of FIG. 9A after five additional population doublings plus 1, 6, or 13 days in culture. These cells covered 20 times the surface area of those in FIG. 9A.

FIG. 9C shows a quantification of the results in FIG. 9A and FIG. 9B by measuring transmitted intensity, which is inversely related to the amount of pigment.

FIG. 10 shows a graph illustrating the effect of cell density at the time of subculture on melanin concentration in cultured human melanocytes, in accordance with embodiments.

FIG. 11 shows spatial distribution of cell densities for human melanocytes cultured on flat or angled (left side of well elevated) culture surfaces, in accordance with embodiments.

FIG. 12A shows a top view schematic of a system for fabricating a patch device, in accordance with embodiments.

FIG. 12B shows a side view schematic of the system of FIG. 12A, in accordance with embodiments.

FIG. 13A shows formation of a patch device mold, in accordance with embodiments.

FIG. 13B shows formation of a gel substrate using the fabricated patch device mold of FIG. 13A, in accordance with embodiments.

FIG. 13C shows formation of a patch device comprising a silicone mesh backing component using patch device mold, in accordance with embodiments.

FIG. 13D shows a side view of the patch device system shown in FIG. 13C during patch device formation, in accordance with embodiments.

FIG. 13E shows removal of the fabricated patch device shown in FIG. 13C from the mold, in accordance with embodiments.

FIG. 13F shows application of adhesive to second surface of patch device, in accordance with embodiments.

FIG. 13G shows application of the patch device shown in FIG. 13F to a target area, in accordance with embodiments.

FIG. 14 shows pigmented melanocytes in target area, in accordance with embodiments.

### DETAILED DESCRIPTION

Disclosed herein are patch devices and methods of use thereof, which are especially suited for the treatment of a skin condition affecting the skin pigmentation of a subject in need thereof (e.g., subjects having a skin condition such as scarring, hypopigmentation, or depigmentation, such as vitiligo).

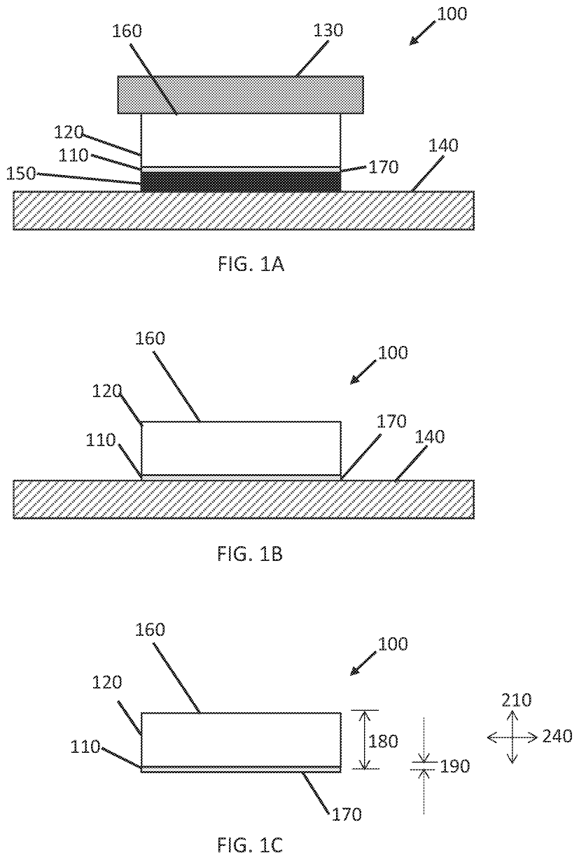

In many embodiments, a patch device comprises a cellular component 110 disposed within a gel substrate 120 of the patch device 100 (e.g., as shown in FIGS. 1A, 1B, and 1C). The cellular component of a patch device comprises a population of cells, in many embodiments. In many cases, the cellular component comprises a population of pigment-producing cells, such as melanocytes. In many cases, cells of the cellular component are useful in treating a skin condition, such as hypopigmentation or depigmentation. In many embodiments, a cellular component comprising a plurality of melanocytes is transferred to a target area (e.g., a hypopigmented or depigmented area) of a subject's skin, using a patch device disclosed herein, for the purpose of repigmenting the target area. In many cases, gel substrate 120 is a hydrogel. Gel substrate 120 comprises fibrin, in many embodiments. In many embodiments, a method of fabricating gel substrate 120 comprises mixing fibrinogen and thrombin to produce fibrin. Gel substrate 120 comprises a first surface 160 and a second surface 170, in some cases. In many cases, second surface 170 of gel substrate 120 is substantially opposite first surface 160 of gel substrate 120. In some cases, a target area of a subject in need of treating (e.g., for a skin condition such as hypopigmentation or depigmentation) is contacted by second surface 160 of gel substrate 120 during treatment, for example, to facilitate transfer of all or a portion of the cells comprising cellular component 110 from the patch device 100 comprising gel substrate 120 to the target area.

In many cases, the gel substrate 120 of patch device 100 maintains the spatial distribution of cells of the cellular component (e.g., melanocytes of a cellular component 110 disposed within the gel substrate 120) during transfer of the cells to a target area of a subject being treated with the patch device. Control over the spatial distribution of the cells comprising the cellular component 110 within gel substrate 120 is achieved by controlling the rate at which gel substrate 120 is formed during fabrication and the physical properties of the final gel substrate (e.g., the mechanical stiffness, and/or the deformability of the gel substrate), in many cases. In some cases, the rate at which gel substrate 120 is formed (e.g., by the enzymatic digestion of fibrinogen to fibrin using thrombin) and the properties of the final gel substrate can be controlled by combining fibrinogen and thrombin in specific ratios at a temperature in a particular range, as described herein. In many cases, controlling spatial distribution of cells within gel substrate 120 (e.g., by controlling the rate at which gel substrate 120 is formed and the properties of the final gel substrate) comprises controlling the spatial distribution of cells within the gel substrate along the vertical dimension (e.g., along the z-axis 210) of the patch device. In some embodiments, a substantial portion of the cells of the cellular component of the cells of the cellular component are disposed in a region of the gel substrate that is at most 20%, at most 10%, or at most 5% of the gel substrate's height 180 from a second surface 170 of the gel substrate (e.g., wherein second surface 170 is brought into contact with a target area of a subject during use of the patch device). For non-limiting example, about 90% of the cells of the cellular component of the gel substrate are within about 10% of the gel's height closest to the second surface 170 in embodiments comprising 10 mg/mL fibrinogen and 2 Units/mL thrombin, about 50% to about 90% of the cells of the cellular component of the gel substrate are within about 10% of the gel's height closest to the second surface 170 in embodiments comprising 15 mg/mL fibrinogen and 2 Units/mL thrombin, about 50% of the cells of the cellular component of the gel substrate are within about 10% of the gel's height closest to the second surface 170 for embodiments comprising 20 mg/mL fibrinogen and 4 Units/mL thrombin, about 50% or about 60% of the cells of the cellular component of the gel substrate are within about 10% of the gel's height closest to the second surface 170 for embodiments comprising 15 mg/mL fibrinogen and 4 Units/mL thrombin, and/or about 50% to about 60% cells of the cellular component of the gel substrate are within about 10% of the gel's height closest to the second surface 170 for embodiments comprising 10 mg/mL fibrinogen and 10 Units/mL thrombin. In some cases, fabricating patch device 100 such that a substantial portion of the cellular component (e.g., 50%-100%, 75%-100%, 80%-100% or 90%-100% of the cellular component) is disposed at a distance of at most 50%, at most 20%, at most 10%, or at most 5% of height 180 results in efficient transfer of cells to the target site.

In many embodiments, the gel substrate maintains the spatial distribution of cells (e.g., pigment-producing cells of the cellular component, such as melanocytes) of a patch device with respect to the length and width of the patch device (e.g., along the x-axis 220 and y-axis 230, respectively, of a cross-sectional x-y plane 240) of the patch device. In many cases, using a patch device 100 comprising gel substrate 120 to transfer cells (e.g., pigment-producing cells such as melanocytes) to a target area of a subject's body (e.g., a hypopigmented or depigmented portion of the subject's skin), as disclosed herein, results in improved control over the spatial distribution of the transferred cells relative to existing technologies that do not maintain spatial distribution of cells (e.g., before cell transfer or during cell transfer). In many embodiments, the spatial distribution of cells of the cellular component 110 of patch device 100 in the vertical dimension (e.g., along the z-axis 210) of the patch device is maintained (e.g., before and/or during transfer of cells to a target area of a subject) by fabricating a gel substrate as disclosed herein. In many cases, maintaining the spatial distribution of cells comprising cellular component 110 (e.g., before and/or during transfer of cells to a target area of a subject) improves the efficiency of transfer of cells from a patch device to a target area of a subject.

In many cases, the distribution of cells of cellular component 110 is determined with respect to an x-y plane 240 of patch device 100. For example, the distribution of cells of cellular component 110 is determined in an x-y plane 240 of gel substrate 120 that is a distance 190 from second surface 170 of patch device 100, in some embodiments. In some cases, distance 190 is 5 percent to 50 percent, 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, 10 percent to 5 percent, or less than 5 percent of a height 180 of the gel substrate. In some embodiments, distance 190 is 50 percent, 45 percent, 40 percent, 35 percent, 30 percent, 25 percent, 20 percent, 15 percent, 10 percent, or 5 percent of a height 180 of the gel substrate. In some embodiments, distance 190 is at least 50 percent, 45 percent, 40 percent, 35 percent, 30 percent, 25 percent, 20 percent, 15 percent, or 10 percent of a height 180 of the gel substrate. In some embodiments, distance 190 is at most 50 percent, at most 45 percent, 40 percent, 35 percent, 30 percent, 25 percent, 20 percent, 15 percent, 10 percent, or 5 percent of a height 180 of the gel substrate. In many cases, the x-y plane in which the distribution of cells of cellular component 110 is determined is substantially parallel to second surface of 170. In some embodiments, the distribution of cells of cellular component 110 is determined in the x-y plane of second surface 170. Determination of distribution of cells of cellular component 110 may be accomplished, for example, by imaging the cellular component of the patch device 100 and placing a grid (at least 3×3) over such image, calculating the percentage of cell coverage in each square, and determining an average number of cells in each square and standard deviation for such average for the patch device, wherein the standard deviation is less than 25% of the average, 20% of the average, less than 15% of the average, less than 10% of the average, or less than 5% of the average. When the devices are created using the methods and teachings herein, the distribution of cells of cellular component 110 may be accomplished, and is reproducible and accurate, by imaging the cellular component of the patch device 100 and placing a grid (3×3, 4×3, 4×4, 4×5, 5×5, 5×6, 6×6, or up to 20×20) over such image, calculating the percentage of cell coverage in at least 5 squares of the grid and determining an average number of cells in each square and standard deviation for such average for the patch device, wherein the standard deviation is less than 25% of the average, 20% of the average, less than 15% of the average, less than 10% of the average, or less than 5% of the average. In some embodiments, the at least 5 squares include a square in an upper left quadrant, a square in an upper right quadrant, a square in a lower left quadrant, a square in a lower right quadrant, and a square in a center section (covering the center of the patch device).

In some cases, a distribution and/or number of cells of cellular component 110 in an x-y plane 240 of patch device 100 disposed within a certain distance from the second surface 170 of the gel substrate 100 is determined using light microscopy. For example, focus of the light microscopy can be tuned in order to determine the number of cells of cellular component 110 that are in focus e.g. cells that are in focus can be the cells that disposed within a certain distance from the second surface 170 of the gel substrate 100.

In some cases, a distribution of cells of cellular component 110 in an x-y plane 240 of patch device 100 is determined using microscopy. For example, a distribution of cells within gel substrate 120 (e.g., a distribution of cells within an x-y plane 240 of gel substrate 120) is determined by evaluating the patch device 100 or a portion thereof using transmitted light microscopy or confocal microscopy, in many cases. In some cases, a distribution of cells within gel substrate 120 (e.g., a distribution of cells within an x-y plane 240 of gel substrate 120) is determined by evaluating visible light microscopy or fluorescence microscopy. In some cases, cells are labeled with a marker prior to evaluation of distribution of cells within gel substrate 120, for example, to aid in quantification of cell number and/or distribution. In some cases, a marker used to label (e.g., stain) cells is a fluorescent marker (e.g., a marker that emits a fluorescent signal when excited by an excitatory wavelength of light, such as a visible light, an infrared light, a fluorescent light, or an ultraviolet light). In some cases, cells or cellular component 110 are labeled after patch device 100 or a portion thereof (e.g., gel substrate 120 comprising cellular component 110) has been manufactured (e.g., fabricated or formed). In some cases, cells of cellular component 110 are labeled before patch device 100 or a portion thereof (e.g., gel substrate 120) has been manufactured (e.g., before the cells of cellular component 110 have been added during the manufacture of patch device 100 or a portion thereof). In some cases, determining the distribution of cells within a patch device or portion thereof comprises labeling (e.g., staining) the cells with a viability dye (e.g., calcein AM). In some cases, a nuclear stain (e.g., 4′,6-diamido-2-phenylindole. Hoechst 33258, Hoechst 33342, or Hoechst 34580) is used to label cells. In some cases, a distribution or density of cells in an x-y plane 240 of patch device 100 is determined by dividing the number of cells or average number of cells counted in the x-y plane by the area of the x-y plane analyzed.

In some embodiments, an x-y plane 240 of patch device 100 or a portion thereof (e.g., an area of gel substrate 120 in x-y plane 240 in which cell distribution is evaluated) comprises 10,000 cells/cm² to 300,000 cells/cm². In some embodiments, an x-y plane 240 of patch device 100 or a portion thereof (e.g., gel substrate 120) comprises 10,000 cells/cm² to 50,000 cells/cm², 10,000 cells/cm² to 75,000 cells/cm², 10,000 cells/cm² to 100,000 cells/cm², 10,000 cells/cm² to 325,000 cells/cm², 10,000 cells/cm² to 150,000 cells/cm², 10,000 cells/cm² to 200,000 cells/cm², 50,000 cells/cm² to 75,000 cells/cm², 50,000 cells/cm² to 300,000 cells/cm², 50,000 cells/cm² to 200,000 cells/cm², 50,000 cells/cm² to 250,000 cells/cm², 50,000 cells/cm² to 250,000 cells/cm², 75,000 cells/cm² to 300,000 cells/cm², 75,000 cells/cm² to 325,000 cells/cm², 75,000 cells/cm² to 250,000 cells/cm², 75,000 cells/cm² to 300,000 cells/cm², 100,000 cells/cm² to 325,000 cells/cm², 100,000 cells/cm² to 250,000 cells/cm², 100,000 cells/cm² to 300,000 cells/cm², 125,000 cells/cm² to 350,000 cells/cm², 125,000 cells/cm² to 300,000 cells/cm², or 150,000 cells/cm² to 300,000 cells/cm². In some embodiments, an x-y plane 240 of patch device 100 or a portion thereof (e.g., gel substrate 120) comprises 10,000 cells/cm², 50,000 cells/cm², 75,000 cells/cm², 100,000 cells/cm², 125,000 cells/cm², 150,000 cells/cm², 175,000 cells/cm², 200,000 cells/cm², 225,000 cells/cm², 230,000 cells/cm², 250,000 cells/cm², 275,000 cells/cm², or 300,000 cells/cm². In some embodiments, an x-y plane 240 of patch device 100 or a portion thereof (e.g., gel substrate 120) comprises at least 10,000 cells/cm², 50,000 cells/cm², 75,000 cells/cm², 100,000 cells/cm², 125,000 cells/cm², 150,000 cells/cm², 175,000 cells/cm², 200,000 cells/cm², 225,000 cells/cm², 250,000 cells/cm², 275,000 cells/cm², 300,000 cells/cm², 325,000 cells/cm², or 350,000 cells/cm². In some embodiments, an x-y plane 240 of patch device 100 or a portion thereof (e.g., gel substrate 120) comprises at most 50,000 cells/cm², 75,000 cells/cm², 100,000 cells/cm², 125,000 cells/cm², 150,000 cells/cm², 175,000 cells/cm², 200,000 cells/cm², 225,000 cells/cm², 250,000 cells/cm², 275,000 cells/cm², or 300,000 cells/cm². In some embodiments the cells are melanocytes.

Manufacturing (e.g., fabricating or forming) a patch device 100 with a distribution of cells in an x-y plane 240 that is within an optimal distribution range disclosed herein confers the unexpected advantages of improving gel degradation rate and increasing efficiency of transfer on the patch device and methods of using the patch device, in many cases. In some cases, a patch device 100 having a cell distribution in an x-y plane 240 that is within an optimal distribution range disclosed herein improves the rate of gel degradation of the patch device by increasing the rate of gel degradation. In some embodiments, an optimal distribution of cells in an x-y plane 240 of patch device 100 or a portion thereof (e.g., an area of gel substrate 120 in x-y plane 240 in which cell distribution is evaluated) comprises 75,000 cells/cm² to 325,000 cells/cm². In some embodiments, an optimal distribution of cells in an x-y plane 240 of patch device 100 or a portion thereof (e.g., gel substrate 120) comprises 75,000 cells/cm² to 280,000 cells/cm², 75,000 cells/cm² to 285,000 cells/cm², 75,000 cells/cm² to 290,000 cells/cm², 75,000 cells/cm² to 295,000 cells/cm², 75,000 cells/cm² to 300,000 cells/cm², 75,000 cells/cm² to 305,000 cells/cm², 75,000 cells/cm² to 310,000 cells/cm², 75,000 cells/cm² to 315,000 cells/cm², 75,000 cells/cm² to 320,000 cells/cm², 75,000 cells/cm² to 325,000 cells/cm², 80,000 cells/cm² to 285,000 cells/cm², 80,000 cells/cm² to 290,000 cells/cm², 80,000 cells/cm² to 295,000 cells/cm², 80,000 cells/cm² to 300,000 cells/cm², 80,000 cells/cm² to 305,000 cells/cm², 80,000 cells/cm² to 310,000 cells/cm², 80,000 cells/cm² to 315,000 cells/cm², 80,000 cells/cm² to 320,000 cells/cm², 80,000 cells/cm² to 325,000 cells/cm², 85,000 cells/cm² to 290,000 cells/cm², 85.000 cells/cm² to 295,000 cells/cm², 85.000 cells/cm² to 300,000 cells/cm², 85,000 cells/cm² to 305,000 cells/cm², 85,000 cells/cm² to 310,000 cells/cm², 85,000 cells/cm² to 315,000 cells/cm², 85.000 cells/cm² to 320,000 cells/cm², 85.000 cells/cm² to 325,000 cells/cm², 90,000 cells/cm² to 295,000 cells/cm², 90,000 cells/cm² to 300,000 cells/cm², 90,000 cells/cm² to 305,000 cells/cm², 90,000 cells/cm² to 310,000 cells/cm², 90,000 cells/cm² to 315,000 cells/cm², 90,000 cells/cm² to 320,000 cells/cm², 90,000 cells/cm² to 325,000 cells/cm², 95,000 cells/cm² to 300,000 cells/cm², 295,000 cells/cm² to 305,000 cells/cm², 95,000 cells/cm² to 310,000 cells/cm², 95,000 cells/cm² to 315,000 cells/cm², 95,000 cells/cm² to 320.000 cells/cm², 95,000 cells/cm² to 325,000 cells/cm², 100,000 cells/cm² to 305,000 cells/cm², 100,000 cells/cm² to 310,000 cells/cm², 100,000 cells/cm² to 315,000 cells/cm², 100,000 cells/cm² to 320,000 cells/cm², 100,000 cells/cm² to 325,000 cells/cm², 105,000 cells/cm² to 310,000 cells/cm², 105,000 cells/cm² to 115,000 cells/cm², 305,000 cells/cm² to 120,000 cells/cm², 105,000 cells/cm² to 325,000 cells/cm², 110,000 cells/cm² to 315,000 cells/cm², 110,000 cells/cm² to 320.000 cells/cm², 110,000 cells/cm² to 325,000 cells/cm², 115,000 cells/cm² to 320,000 cells/cm², 115,000 cells/cm² to 325,000 cells/cm², or 120,000 cells/cm² to 325,000 cells/cm². In some embodiments, an optimal distribution of cells in an x-y plane 240 of patch device 100 or a portion thereof (e.g., gel substrate 120) comprises 75,000 cells/cm², 80,000 cells/cm², 85,000 cells/cm², 90,000 cells/cm², 95,000 cells/cm², 100,000 cells/cm², 105,000 cells/cm², 110,000 cells/cm², 115,000 cells/cm², 120,000 cells/cm², 125,000 cells/cm², 130,000 cells/cm², 140,000 cells/cm², 145,000 cells/cm², 150,000 cells/cm², 155,000 cells/cm², 160,000 cells/cm², 165,000 cells/cm², 170,000 cells/cm², 175,000 cells/cm², 180,000 cells/cm². 185,000 cells/cm², 190,000 cells/cm², 195,000 cells/cm², 200,000 cells/cm², 205,000 cells/cm², 210,000 cells/cm², 215,000 cells/cm², 220,000 cells/cm², 225,000 cells/cm², 230,000 cells/cm², 235,000 cells/cm², 240,000 cells/cm², 245,000 cells/cm², 250,000 cells/cm², 255,000 cells/cm², 260,000 cells/cm², 265,000 cells/cm², 270,000 cells/cm², 275,000 cells/cm², 280,000 cells/cm², 285,000 cells/cm², 290,000 cells/cm², 295,000 cells/cm², or 300,000 cells/cm². In some embodiments, an optimal distribution of cells in an x-y plane 240 of patch device 100 or a portion thereof (e.g., gel substrate 120) comprises at least 75,000 cells/cm², 80,000 cells/cm², 85,000 cells/cm², 90,000 cells/cm², 95,000 cells/cm², 100,000 cells/cm², 105,000 cells/cm², 110,000 cells/cm², 115,000 cells/cm², 120,000 cells/cm², 125,000 cells/cm², 130,000 cells/cm², 135,000 cells/cm², 140,000 cells/cm², 145,000 cells/cm², 150,000 cells/cm², 155,000 cells/cm², 160,000 cells/cm², 165,000 cells/cm², 170,000 cells/cm², 175,000 cells/cm², 180,000 cells/cm², 185,000 cells/cm², 190,000 cells/cm², 195,000 cells/cm², 200,000 cells/cm², 205,000 cells/cm², 210,000 cells/cm², 215,000 cells/cm², 220,000 cells/cm², 225,000 cells/cm², 230,000 cells/cm², 235,000 cells/cm², 240,000 cells/cm², 245,000 cells/cm², 250,000 cells/cm², 255,000 cells/cm², 260,000 cells/cm², 265,000 cells/cm², 270,000 cells/cm², 275,000 cells/cm², 280,000 cells/cm², 285,000 cells/cm², 290,000 cells/cm², 295,000 cells/cm², 300,000 cells/cm², 305,000 cells/cm², 310,000 cells/cm², 315,000 cells/cm², or 320.000 cells/cm². In some embodiments, an optimal distribution of cells in an x-y plane 240 of patch device 100 or a portion thereof (e.g., gel substrate 120) comprises at most 80,000 cells/cm², 85,000 cells/cm², 90,000 cells/cm², 95,000 cells/cm², 100,000 cells/cm², 105,000 cells/cm², 110,000 cells/cm², 115,000 cells/cm², 120,000 cells/cm², 125,000 cells/cm², 130,000 cells/cm², 135,000 cells/cm², 140,000 cells/cm², 145,000 cells/cm², 150,000 cells/cm², 155,000 cells/cm², 160,000 cells/cm², 165,000 cells/cm², 170,000 cells/cm², 175,000 cells/cm². 180,000 cells/cm², 185,000 cells/cm², 190,000 cells/cm², 195,000 cells/cm², 200,000 cells/cm², 205,000 cells/cm², 210,000 cells/cm², 215,000 cells/cm², 220,000 cells/cm², 225,000 cells/cm², 230,000 cells/cm², 235,000 cells/cm², 240,000 cells/cm², 245,000 cells/cm², 250,000 cells/cm², 255,000 cells/cm², 260,000 cells/cm², 265,000 cells/cm², 270,000 cells/cm², 275,000 cells/cm², 280,000 cells/cm², 285,000 cells/cm², 290,000 cells/cm², 295,000 cells/cm², 300,000 cells/cm², 305,000 cells/cm², 310,000 cells/cm², 315,000 cells/cm², 320,000 cells/cm², or 325,000 cells/cm². In some embodiments, the cells are melanocytes.

In many cases, the thickness of an x-y plane 240 in which cell distribution (e.g., number of cells per unit area in the plane) is evaluated is 1 micrometer to 2,000 micrometers. In many cases, the thickness of an x-y plane 240 in which cell distribution (e.g., number of cells per unit area in the plane) is evaluated is 1 micrometer to 10 micrometers, 1 micrometer to 20 micrometers, 1 micrometer to 40 micrometers, 1 micrometer to 60 micrometers, 1 micrometer to 80 micrometers, 1 micrometer to 100 micrometers, 1 micrometer to 200 micrometers, 1 micrometer to 500 micrometers, 1 micrometer to 1,000 micrometers, 1 micrometer to 2,000 micrometers, 10 micrometers to 20 micrometers, 10 micrometers to 40 micrometers, 10 micrometers to 60 micrometers, 10 micrometers to 80 micrometers, 10 micrometers to 100 micrometers, 10 micrometers to 200 micrometers, 10 micrometers to 500 micrometers, 10 micrometers to 1,000 micrometers, 10 micrometers to 2,000 micrometers, 20 micrometers to 40 micrometers, 20 micrometers to 60 micrometers, 20 micrometers to 80 micrometers, 20 micrometers to 100 micrometers, 20 micrometers to 200 micrometers, 20 micrometers to 500 micrometers, 20 micrometers to 1,000 micrometers, 20 micrometers to 2,000 micrometers, 40 micrometers to 60 micrometers, 40 micrometers to 80 micrometers, 40 micrometers to 100 micrometers, 40 micrometers to 200 micrometers, 40 micrometers to 500 micrometers, 40 micrometers to 1,000 micrometers, 40 micrometers to 2,000 micrometers, 60 micrometers to 80 micrometers, 60 micrometers to 100 micrometers, 60 micrometers to 200 micrometers, 60 micrometers to 500 micrometers, 60 micrometers to 1,000 micrometers, 60 micrometers to 2,000 micrometers, 80 micrometers to 100 micrometers, 80 micrometers to 200 micrometers, 80 micrometers to 500 micrometers, 80 micrometers to 1,000 micrometers, 80 micrometers to 2,000 micrometers, 100 micrometers to 200 micrometers, 100 micrometers to 500 micrometers, 100 micrometers to 1,000 micrometers, 100 micrometers to 2,000 micrometers, 200 micrometers to 500 micrometers, 200 micrometers to 1,000 micrometers, 200 micrometers to 2,000 micrometers, 500 micrometers to 1,000 micrometers, 500 micrometers to 2,000 micrometers, or 1,000 micrometers to 2,000 micrometers. In many cases, the thickness of an x-y plane 240 in which cell distribution (e.g., number of cells per unit area in the plane) is evaluated is 1 micrometer, 10 micrometers, 20 micrometers, 40 micrometers, 60 micrometers, 80 micrometers, 100 micrometers, 200 micrometers, 500 micrometers, 1,000 micrometers, or 2,000 micrometers. In many cases, the thickness of an x-y plane 240 in which cell distribution (e.g., number of cells per unit area in the plane) is evaluated is at least 1 micrometer, 10 micrometers, 20 micrometers, 40 micrometers, 60 micrometers, 80 micrometers, 100 micrometers, 200 micrometers, 500 micrometers, 1,000 micrometers, or at least 2,000 micrometers. In many cases, the thickness of an x-y plane 240 in which cell distribution (e.g., number of cells per unit area in the plane) is evaluated is at most 10 micrometers, 20 micrometers, 40 micrometers, 60 micrometers, 80 micrometers, 100 micrometers, 200 micrometers, 500 micrometers, 1,000 micrometers, or 2,000 micrometers.

In many cases, the thickness of an x-y plane 240 in which cell distribution (e.g., number of cells per unit area in the plane) is 75,000 cells/cm² to 325,000 cells/cm² is 1 micrometer to 2,000 micrometers. In many cases, the thickness of an x-y plane 240 in which cell distribution (e.g., number of cells per unit area in the plane) is at least any of the minimum concentrations (cells/cm²) noted herein, is within any of the concentration ranges (cells/cm²) noted herein, or is at most any of the maximum concentrations (cells/cm²) is 1 micrometer to 2,000 micrometers. In many cases, the thickness of an x-y plane 240 in which cell distribution (e.g., number of cells per unit area in the plane) is 75,000 cells/cm² to 325,000 cells/cm² is 1 micrometer to 10 micrometers, 1 micrometer to 20 micrometers, 1 micrometer to 40 micrometers, 1 micrometer to 60 micrometers, 1 micrometer to 80 micrometers, 1 micrometer to 100 micrometers, 1 micrometer to 200 micrometers, 1 micrometer to 500 micrometers, 1 micrometer to 1,000 micrometers, 1 micrometer to 2,000 micrometers, 10 micrometers to 20 micrometers, 10 micrometers to 40 micrometers, 10 micrometers to 60 micrometers, 10 micrometers to 80 micrometers, 10 micrometers to 100 micrometers, 10 micrometers to 200 micrometers, 10 micrometers to 500 micrometers, 10 micrometers to 1,000 micrometers, 10 micrometers to 2,000 micrometers, 20 micrometers to 40 micrometers, 20 micrometers to 60 micrometers, 20 micrometers to 80 micrometers, 20 micrometers to 100 micrometers, 20 micrometers to 200 micrometers, 20 micrometers to 500 micrometers, 20 micrometers to 1,000 micrometers, 20 micrometers to 2,000 micrometers, 40 micrometers to 60 micrometers, 40 micrometers to 80 micrometers, 40 micrometers to 100 micrometers, 40 micrometers to 200 micrometers, 40 micrometers to 500 micrometers, 40 micrometers to 1,000 micrometers, 40 micrometers to 2,000 micrometers, 60 micrometers to 80 micrometers, 60 micrometers to 100 micrometers, 60 micrometers to 200 micrometers, 60 micrometers to 500 micrometers, 60 micrometers to 1,000 micrometers, 60 micrometers to 2,000 micrometers, 80 micrometers to 100 micrometers, 80 micrometers to 200 micrometers, 80 micrometers to 500 micrometers, 80 micrometers to 1,000 micrometers, 80 micrometers to 2,000 micrometers, 100 micrometers to 200 micrometers, 100 micrometers to 500 micrometers, 100 micrometers to 1,000 micrometers, 100 micrometers to 2,000 micrometers, 200 micrometers to 500 micrometers, 200 micrometers to 1,000 micrometers, 200 micrometers to 2,000 micrometers, 500 micrometers to 1,000 micrometers, 500 micrometers to 2,000 micrometers, or 1,000 micrometers to 2,000 micrometers. In many cases, the thickness of an x-y plane 240 in which cell distribution (e.g., number of cells per unit area in the plane) is at least any of the minimum concentrations (cells/cm²) noted herein, is within any of the concentration ranges (cells/cm²) noted herein, or is at most any of the maximum concentrations (cells/cm²) is 1 micrometer to 10 micrometers, 1 micrometer to 20 micrometers, 1 micrometer to 40 micrometers, 1 micrometer to 60 micrometers, 1 micrometer to 80 micrometers, 1 micrometer to 100 micrometers, 1 micrometer to 200 micrometers, 1 micrometer to 500 micrometers, 1 micrometer to 1,000 micrometers, 1 micrometer to 2,000 micrometers, 10 micrometers to 20 micrometers, 10 micrometers to 40 micrometers, 10 micrometers to 60 micrometers, 10 micrometers to 80 micrometers, 10 micrometers to 100 micrometers, 10 micrometers to 200 micrometers, 10 micrometers to 500 micrometers, 10 micrometers to 1,000 micrometers, 10 micrometers to 2,000 micrometers, 20 micrometers to 40 micrometers, 20 micrometers to 60 micrometers, 20 micrometers to 80 micrometers, 20 micrometers to 100 micrometers, 20 micrometers to 200 micrometers, 20 micrometers to 500 micrometers, 20 micrometers to 1,000 micrometers, 20 micrometers to 2,000 micrometers, 40 micrometers to 60 micrometers, 40 micrometers to 80 micrometers, 40 micrometers to 100 micrometers, 40 micrometers to 200 micrometers, 40 micrometers to 500 micrometers, 40 micrometers to 1,000 micrometers, 40 micrometers to 2,000 micrometers, 60 micrometers to 80 micrometers, 60 micrometers to 100 micrometers, 60 micrometers to 200 micrometers, 60 micrometers to 500 micrometers, 60 micrometers to 1,000 micrometers, 60 micrometers to 2,000 micrometers, 80 micrometers to 100 micrometers, 80 micrometers to 200 micrometers, 80 micrometers to 500 micrometers, 80 micrometers to 1,000 micrometers, 80 micrometers to 2,000 micrometers, 100 micrometers to 200 micrometers, 100 micrometers to 500 micrometers, 100 micrometers to 1,000 micrometers, 100 micrometers to 2,000 micrometers, 200 micrometers to 500 micrometers, 200 micrometers to 1,000 micrometers, 200 micrometers to 2,000 micrometers, 500 micrometers to 1,000 micrometers, 500 micrometers to 2,000 micrometers, or 1,000 micrometers to 2,000 micrometers. In many cases, the thickness of an x-y plane 240 in which cell distribution (e.g., number of cells per unit area in the plane) is 75,000 cells/cm² to 325,000 cells/cm² is 1 micrometer, 10 micrometers, 20 micrometers, 40 micrometers, 60 micrometers, 80 micrometers, 100 micrometers, 200 micrometers, 500 micrometers, 1,000 micrometers, or 2,000 micrometers. In many cases, the thickness of an x-y plane 240 in which cell distribution (e.g., number of cells per unit area in the plane) is 75,000 cells/cm² to 325,000 cells/cm² is at least 1 micrometer, 10 micrometers, 20 micrometers, 40 micrometers, 60 micrometers, 80 micrometers, 100 micrometers, 200 micrometers, 500 micrometers, 1,000 micrometers, or at least 2,000 micrometers. In many cases, the thickness of an x-y plane 240 in which cell distribution (e.g., number of cells per unit area in the plane) is 75,000 cells/cm² to 325,000 cells/cm² is at most 10 micrometers. In many cases, the thickness of an x-y plane 240 in which cell distribution (e.g., number of cells per unit area in the plane) is 75,000 cells/cm² to 325,000 cells/cm² is at most 20 micrometers. In many cases, the thickness of an x-y plane 240 in which cell distribution (e.g., number of cells per unit area in the plane) is 75,000 cells/cm² to 325,000 cells/cm² is at most 40 micrometers. In many cases, the thickness of an x-y plane 240 in which cell distribution (e.g., number of cells per unit area in the plane) is 75,000 cells/cm² to 325,000 cells/cm² is at most 60 micrometers. In many cases, the thickness of an x-y plane 240 in which cell distribution (e.g., number of cells per unit area in the plane) is 75,000 cells/cm² to 325,000 cells/cm² is at most 80 micrometers. In many cases, the thickness of an x-y plane 240 in which cell distribution (e.g., number of cells per unit area in the plane) is 75,000 cells/cm² to 325,000 cells/cm² is at most 100 micrometers. In many cases, the thickness of an x-y plane 240 in which cell distribution (e.g., number of cells per unit area in the plane) is 75,000 cells/cm² to 325,000 cells/cm² is at most 200 micrometers. In many cases, the thickness of an x-y plane 240 in which cell distribution (e.g., number of cells per unit area in the plane) is 75,000 cells/cm² to 325,000 cells/cm² is at most 500 micrometers. In many cases, the thickness of an x-y plane 240 in which cell distribution (e.g., number of cells per unit area in the plane) is 75,000 cells/cm² to 325,000 cells/cm² is at most 1,000 micrometers. In many cases, the thickness of an x-y plane 240 in which cell distribution (e.g., number of cells per unit area in the plane) is 75,000 cells/cm² to 325,000 cells/cm² is at most 2,000 micrometers.

In many cases, the area of an x-y plane 240 in which cell distribution (e.g., number of cells per unit area in the plane) is evaluated is 0.001 square millimeters to 400 square millimeters. In many cases, the area of an x-y plane 240 in which cell distribution (e.g., number of cells per unit area in the plane) is evaluated is 0.001 square millimeters to 0.01 square millimeters, 0.001 square millimeters to 0.1 square millimeters, 0.001 square millimeters to 1 square millimeter, 0.001 square millimeters to 10 square millimeters, 0.001 square millimeters to 100 square millimeters, 0.001 square millimeters to 200 square millimeters, 0.001 square millimeters to 400 square millimeters, 0.01 square millimeters to 0.1 square millimeters, 0.01 square millimeters to 1 square millimeter, 0.01 square millimeters to 10 square millimeters, 0.01 square millimeters to 100 square millimeters, 0.01 square millimeters to 200 square millimeters, 0.01 square millimeters to 400 square millimeters, 0.1 square millimeters to 1 square millimeter, 0.1 square millimeters to 10 square millimeters, 0.1 square millimeters to 100 square millimeters, 0.1 square millimeters to 200 square millimeters, 0.1 square millimeters to 400 square millimeters, 1 square millimeter to 10 square millimeters, 1 square millimeter to 100 square millimeters, 1 square millimeter to 200 square millimeters, 1 square millimeter to 400 square millimeters, 10 square millimeters to 100 square millimeters, 10 square millimeters to 200 square millimeters, 10 square millimeters to 400 square millimeters, 100 square millimeters to 200 square millimeters, 100 square millimeters to 400 square millimeters, or 200 square millimeters to 400 square millimeters. In many cases, the area of an x-y plane 240 in which cell distribution (e.g., number of cells per unit area in the plane) is evaluated is 0.001 square millimeters, 0.01 square millimeters, 0.1 square millimeters, 1 square millimeter, 10 square millimeters, 100 square millimeters, 200 square millimeters, or 400 square millimeters. In many cases, the area of an x-y plane 240 in which cell distribution (e.g., number of cells per unit area in the plane) is evaluated is at least 0.001 square millimeters, 0.01 square millimeters, 0.1 square millimeters, 1 square millimeter, 10 square millimeters, 100 square millimeters, 200 square millimeters, or at least 400 square millimeters. In many cases, the area of an x-y plane 240 in which cell distribution (e.g., number of cells per unit area in the plane) is evaluated is at most 0.01 square millimeters, 0.1 square millimeters, 1 square millimeter, 10 square millimeters, 100 square millimeters, 200 square millimeters, or 400 square millimeters.

In some embodiments, treating a patient with patch device 100 results in a spatial distribution of transferred cells (e.g., a population of cells comprising pigment-producing cells of cellular component 110) of 75,000 cells/cm² to 325,000 cells/cm² at the target area of the skin of the subject. In some embodiments, treating a patient with patch device 100 results in a spatial distribution of transferred cells (e.g., a population of cells comprising pigment-producing cells of cellular component 110) of at least any of the minimum concentrations (cells/cm²) noted herein, of within any of the concentration ranges (cells/cm²) noted herein, or of at most any of the maximum concentrations (cells/cm²) at the target area of the skin of the subject. In some embodiments, treating a patient with patch device 100 results in a spatial distribution of transferred cells (e.g., a population of cells comprising pigment-producing cells of cellular component 110) of 75,000 cells/cm² to 80,000 cells/cm², 75,000 cells/cm² to 85,000 cells/cm², 75,000 cells/cm² to 90,000 cells/cm², 75,000 cells/cm² to 95,000 cells/cm², 75,000 cells/cm² to 100,000 cells/cm², 75,000 cells/cm² to 105,000 cells/cm², 75,000 cells/cm² to 110,000 cells/cm², 75,000 cells/cm² to 115,000 cells/cm², 75,000 cells/cm² to 120,000 cells/cm², 75,000 cells/cm² to 325,000 cells/cm², 80,000 cells/cm² to 85,000 cells/cm², 80,000 cells/cm² to 90,000 cells/cm², 80,000 cells/cm² to 95,000 cells/cm², 80,000 cells/cm² to 100,000 cells/cm², 80,000 cells/cm² to 105,000 cells/cm², 80,000 cells/cm² to 110,000 cells/cm², 80,000 cells/cm² to 115,000 cells/cm², 80,000 cells/cm² to 120,000 cells/cm², 80,000 cells/cm² to 325,000 cells/cm², 85,000 cells/cm² to 90,000 cells/cm², 85,000 cells/cm² to 95,000 cells/cm², 85.000 cells/cm² to 100,000 cells/cm², 85.000 cells/cm² to 105,000 cells/cm², 85.000 cells/cm² to 110,000 cells/cm², 85,000 cells/cm² to 115,000 cells/cm², 85,000 cells/cm² to 120,000 cells/cm², 85,000 cells/cm² to 325,000 cells/cm², 90.00) cells/cm² to 95,000 cells/cm², 90,000 cells/cm² to 100,000 cells/cm², 90,000 cells/cm² to 105,000 cells/cm², 90,000 cells/cm² to 110,000 cells/cm², 90,000 cells/cm² to 115,000 cells/cm², 90,000 cells/cm² to 120,000 cells/cm², 90.00) cells/cm² to 325,000 cells/cm², 95,000 cells/cm² to 100,000 cells/cm², 95,000 cells/cm² to 105,000 cells/cm², 95,000 cells/cm² to 110,000 cells/cm², 95,000 cells/cm² to 115,000 cells/cm², 95,000 cells/cm² to 120,000 cells/cm², 95,000 cells/cm² to 325,000 cells/cm², 100,000 cells/cm² to 105,000 cells/cm², 100,000 cells/cm² to 110,000 cells/cm², 100,000 cells/cm² to 115,000 cells/cm², 100,000 cells/cm² to 120,000 cells/cm², 100,000 cells/cm² to 325,000 cells/cm², 105,000 cells/cm² to 110,000 cells/cm², 105,000 cells/cm² to 115,000 cells/cm², 105,000 cells/cm² to 120,000 cells/cm², 105,000 cells/cm² to 325,000 cells/cm², 110,000 cells/cm² to 115,000 cells/cm², 110,000 cells/cm² to 120,000 cells/cm², 110,000 cells/cm² to 325,000 cells/cm², 115,000 cells/cm² to 120,000 cells/cm², 115,000 cells/cm² to 325,000 cells/cm², or 120,000 cells/cm² to 325,000 cells/cm² at the target area of the skin of the subject. In some embodiments, treating a patient with patch device 100 results in a spatial distribution of transferred cells (e.g., a population of cells comprising pigment-producing cells of cellular component 110) of 75,000 cells/cm², 80,000 cells/cm², 85.000 cells/cm², 90,000 cells/cm², 95,000 cells/cm², 100,000 cells/cm², 105,000 cells/cm², 110,000 cells/cm², 115,000 cells/cm², 120,000 cells/cm², or 325,000 cells/cm² at the target area of the skin of the subject. In some embodiments, treating a patient with patch device 100 results in a spatial distribution of transferred cells (e.g., a population of cells comprising pigment-producing cells of cellular component 110) of at least 75,000 cells/cm², 80,000 cells/cm², 85,000 cells/cm², 90,000 cells/cm², 95,000 cells/cm², 100,000 cells/cm², 105,000 cells/cm², 110,000 cells/cm², 115,000 cells/cm², 120,000 cells/cm², or 325,000 cells/cm² at the target area of the skin of the subject. In some embodiments, treating a patient with patch device 100 results in a spatial distribution of transferred cells (e.g., a population of cells comprising pigment-producing cells of cellular component 110) of at most 80,000 cells/cm². 85,000 cells/cm², 90,000 cells/cm², 95,000 cells/cm², 100,000 cells/cm², 105,000 cells/cm², 110,000 cells/cm², 115,000 cells/cm², 120,000 cells/cm², or 325,000 cells/cm² at the target area of the skin of the subject.

The mechanical stiffness (e.g., Young's modulus in compression) of a gel substrate 120 can be increased by changing the composition of the gel substrate. For example, increasing the concentration of fibrinogen that is mixed with a given amount of thrombin can increase the mechanical stiffness of the final patch device. In some cases, increasing the concentration of thrombin that is mixed with a given amount of thrombin can increase the mechanical stiffness of the final patch device. In some cases (e.g., at high thrombin concentrations, such as concentrations greater than about 20 U/mL to about 50 U/mL), further increasing thrombin concentration mixed with a given concentration of fibrinogen (e.g., 5 mg/mL to 15 mg/mL fibrinogen) can decrease mechanical stiffness of the final patch device. In some embodiments, gel substrate 120 has a Young's modulus in compression of 0.1 kPa to 20 kPa. In some cases, the mechanical stiffness of a gel substrate 120 can be tuned by changing the composition of the gel substrate to target a specific tissue (e.g., a portion of a knee, elbow, or arm).

In some embodiments, gel substrate 120 has a Young's modulus in compression of 0.1 kPa to 0.5 kPa, 0.1 kPa to 1 kPa, 0.1 kPa to 2 kPa, 0.1 kPa to 5 kPa, 0.1 kPa to 6 kPa, 0.1 kPa to 7.5 kPa, 0.1 kPa to 8.5 kPa, 0.1 kPa to 10 kPa, 0.1 kPa to 14 kPa, 0.1 kPa to 17 kPa, 0.1 kPa to 20 kPa, 0.5 kPa to 1 kPa, 0.5 kPa to 2 kPa, 0.5 kPa to 5 kPa, 0.5 kPa to 6 kPa, 0.5 kPa to 7.5 kPa, 0.5 kPa to 8.5 kPa, 0.5 kPa to 10 kPa, 0.5 kPa to 14 kPa, 0.5 kPa to 17 kPa, 0.5 kPa to 20 kPa, 1 kPa to 2 kPa, 1 kPa to 5 kPa, 1 kPa to 6 kPa, 1 kPa to 7.5 kPa, 1 kPa to 8.5 kPa, 1 kPa to 10 kPa, 1 kPa to 14 kPa, 1 kPa to 17 kPa, 1 kPa to 20 kPa, 2 kPa to 5 kPa, 2 kPa to 6 kPa, 2 kPa to 7.5 kPa, 2 kPa to 8.5 kPa, 2 kPa to 10 kPa, 2 kPa to 14 kPa, 2 kPa to 17 kPa, 2 kPa to 20 kPa, 5 kPa to 6 kPa, 5 kPa to 7.5 kPa, 5 kPa to 8.5 kPa, 5 kPa to 10 kPa, 5 kPa to 14 kPa, 5 kPa to 17 kPa, 5 kPa to 20 kPa, 6 kPa to 7.5 kPa, 6 kPa to 8.5 kPa, 6 kPa to 10 kPa, 6 kPa to 14 kPa, 6 kPa to 17 kPa, 6 kPa to 20 kPa, 7.5 kPa to 8.5 kPa, 7.5 kPa to 10 kPa, 7.5 kPa to 14 kPa, 7.5 kPa to 17 kPa, 7.5 kPa to 20 kPa, 8.5 kPa to 10 kPa, 8.5 kPa to 14 kPa, 8.5 kPa to 17 kPa, 8.5 kPa to 20 kPa, 10 kPa to 14 kPa, 10 kPa to 17 kPa, 10 kPa to 20 kPa, 14 kPa to 17 kPa, 14 kPa to 20 kPa, or 17 kPa to 20 kPa. In some embodiments, gel substrate has a Young's modulus in compression of 0.1 kPa, 0.5 kPa, 1 kPa, 2 kPa, 5 kPa, 6 kPa, 7.5 kPa, 8.5 kPa, 10 kPa, 14 kPa, 17 kPa, or 20 kPa. In some embodiments, gel substrate has a Young's modulus in compression of at least 0.1 kPa, 0.5 kPa, 1 kPa, 2 kPa, 5 kPa, 6 kPa, 7.5 kPa, 8.5 kPa, 10 kPa, 14 kPa, or 17 kPa. In some embodiments, gel substrate has a Young's modulus in compression of at most 0.5 kPa, 1 kPa, 2 kPa, 5 kPa, 6 kPa, 7.5 kPa, 8.5 kPa, 10 kPa, 14 kPa, 17 kPa, or 20 kPa.

In some embodiments, gel substrate has a Young's modulus in compression of 0.1 kPa to 20 kPa, wherein the gel substrate is formed using 10 mg/mL fibrinogen and 2 U/mL thrombin. In some embodiments, gel substrate has a Young's modulus in compression of 0.1 kPa to 0.5 kPa, 0.1 kPa to 1 kPa, 0.1 kPa to 2 kPa, 0.1 kPa to 5 kPa, 0.1 kPa to 6 kPa, 0.1 kPa to 7.5 kPa, 0.1 kPa to 8.5 kPa, 0.1 kPa to 10 kPa, 0.1 kPa to 14 kPa, 0.1 kPa to 17 kPa, 0.1 kPa to 20 kPa, 0.5 kPa to 1 kPa, 0.5 kPa to 2 kPa, 0.5 kPa to 5 kPa, 0.5 kPa to 6 kPa, 0.5 kPa to 7.5 kPa, 0.5 kPa to 8.5 kPa, 0.5 kPa to 10 kPa, 0.5 kPa to 14 kPa, 0.5 kPa to 17 kPa, 0.5 kPa to 20 kPa, 1 kPa to 2 kPa, 1 kPa to 5 kPa, 1 kPa to 6 kPa, 1 kPa to 7.5 kPa, 1 kPa to 8.5 kPa, 1 kPa to 10 kPa, 1 kPa to 14 kPa, 1 kPa to 17 kPa, 1 kPa to 20 kPa, 2 kPa to 5 kPa, 2 kPa to 6 kPa, 2 kPa to 7.5 kPa, 2 kPa to 8.5 kPa, 2 kPa to 10 kPa, 2 kPa to 14 kPa, 2 kPa to 17 kPa, 2 kPa to 20 kPa, 5 kPa to 6 kPa, 5 kPa to 7.5 kPa, 5 kPa to 8.5 kPa, 5 kPa to 10 kPa, 5 kPa to 14 kPa, 5 kPa to 17 kPa, 5 kPa to 20 kPa, 6 kPa to 7.5 kPa, 6 kPa to 8.5 kPa, 6 kPa to 10 kPa, 6 kPa to 14 kPa, 6 kPa to 17 kPa, 6 kPa to 20 kPa, 7.5 kPa to 8.5 kPa, 7.5 kPa to 10 kPa, 7.5 kPa to 14 kPa, 7.5 kPa to 17 kPa, 7.5 kPa to 20 kPa, 8.5 kPa to 10 kPa, 8.5 kPa to 14 kPa, 8.5 kPa to 17 kPa, 8.5 kPa to 20 kPa, 10 kPa to 14 kPa, 10 kPa to 17 kPa, 10 kPa to 20 kPa, 14 kPa to 17 kPa, 14 kPa to 20 kPa, or 17 kPa to 20 kPa, wherein the gel substrate is formed using 10 mg/mL fibrinogen and 2 U/mL thrombin. In some embodiments, gel substrate has a Young's modulus in compression of 0.1 kPa, 0.5 kPa, 1 kPa, 2 kPa, 5 kPa, 6 kPa, 7.5 kPa, 8.5 kPa, 10 kPa, 14 kPa, 17 kPa, or 20 kPa, wherein the gel substrate is formed using 10 mg/mL fibrinogen and 2 U/mL thrombin. In some embodiments, gel substrate has a Young's modulus in compression of at least 0.1 kPa, 0.5 kPa, 1 kPa, 2 kPa, 5 kPa, 6 kPa, 7.5 kPa, 8.5 kPa, 10 kPa, 14 kPa, or 17 kPa, wherein the gel substrate is formed using 10 mg/mL fibrinogen and 2 U/mL thrombin. In some embodiments, gel substrate has a Young's modulus in compression of at most 0.5 kPa, 1 kPa, 2 kPa, 5 kPa, 6 kPa, 7.5 kPa, 8.5 kPa, 10 kPa, 14 kPa, 17 kPa, or 20 kPa, wherein the gel substrate is formed using 10 mg/mL fibrinogen and 2 U/mL thrombin.

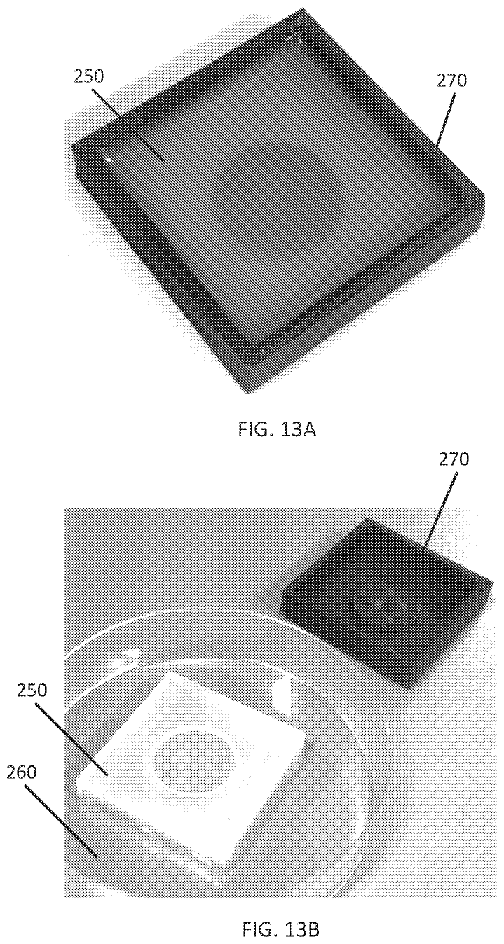

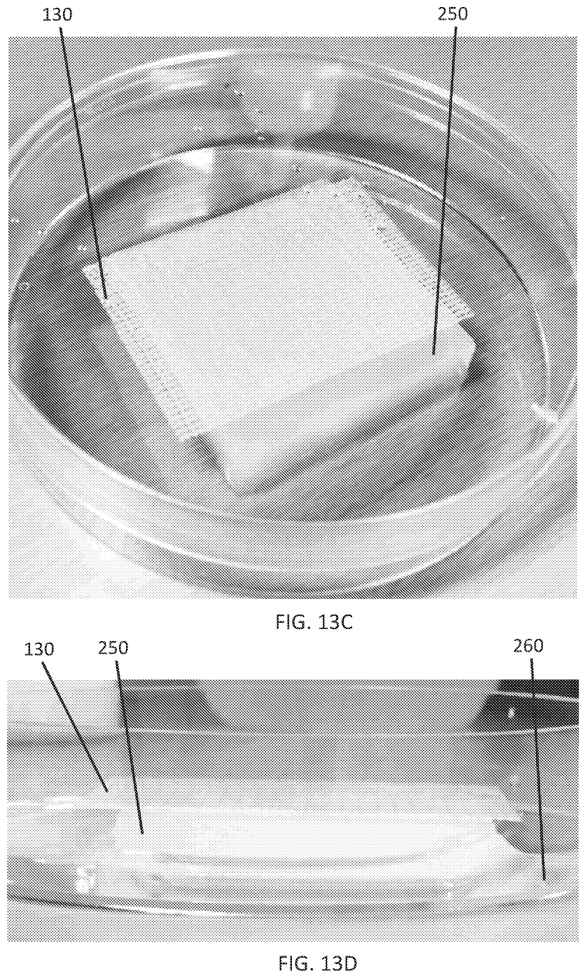

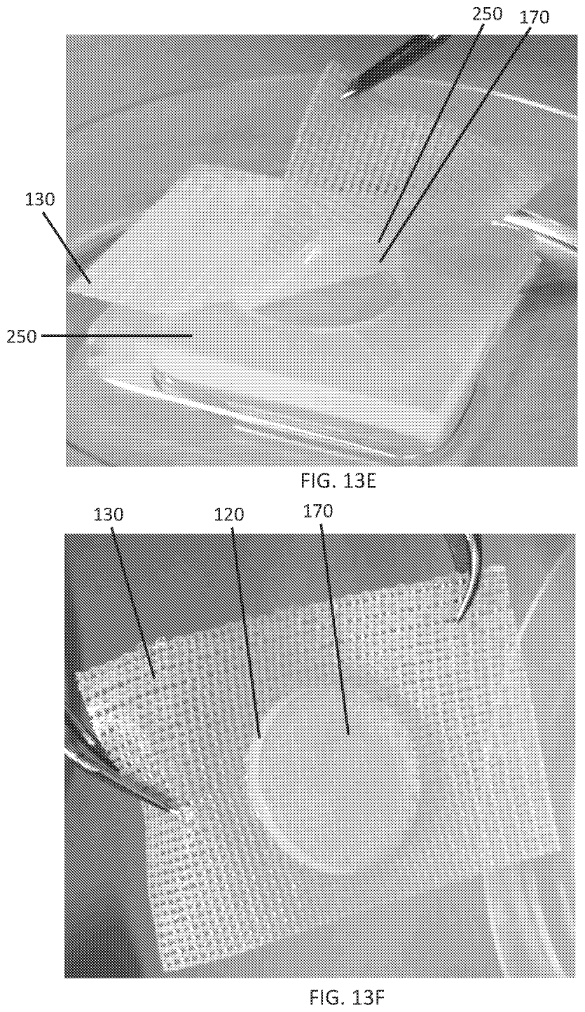

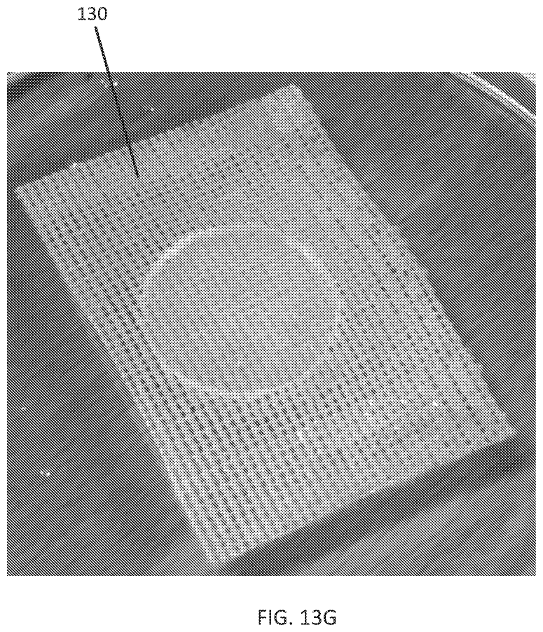

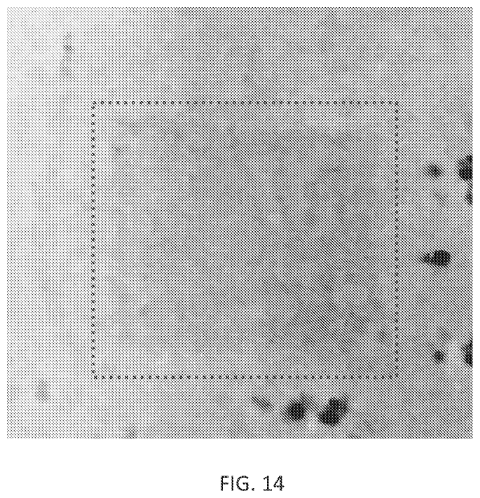

Patch devices disclosed herein comprise a backing component 130 coupled to the gel substrate 120, in many embodiments. In many cases, a patch device comprising a backing component 130 improves the ability of a practitioner to manipulate patch device 100 (e.g., before or during application of the patch device to a target area of a subject). In some embodiments, a practitioner (e.g., a technician or physician) removes a patch device from a mold 250 or container by handling the backing component 130 of the patch device (e.g., using forceps or tweezers), as shown in FIG. 13E. In some cases, a practitioner can manipulate the patch device by grasping the backing component (e.g., using forceps or tweezers) for the addition of an adhesive to second surface 170 or for placement of the patch device at a target area, for example, as shown in FIG. 13F. FIG. 13G shows a patch device comprising a backing component 130 that has been manipulated into position at a target area and applied to the target area such that the second surface of the patch device is in contact with the target area and the backing component 130 is facing up. In some cases, the backing component 130 can be removed (e.g., manually removed) from a gel substrate 120 after the patch device is applied to a target area of a subject. In many cases, a backing component 130 of a patch device 100 has a higher stiffness than that of a gel substrate 120 to which it is coupled. A backing component 130 of a patch device is formed by enzymatically digesting fibrinogen into fibrin using thrombin, in some embodiments. In some cases, backing component comprises a polymer (e.g., a polymeric sheet or dressing, such as a silicone mesh). In some cases, the mechanical stiffness of a backing component of a patch device is 50% to 100%, 100% to 200%, 200% to 300%, 300% to 400%, or more than 400% higher than that of the gel substrate of the patch device. In some cases, the mechanical stiffness of a backing component 130 of a patch device 100 is 10 percent to 400 percent higher than the mechanical stiffness of the gel substrate 120 of the patch device. In some cases, the mechanical stiffness of a backing component of a patch device is 10 percent to 50 percent, 10 percent to 100 percent, 10 percent to 200 percent, 10 percent to 300 percent, 10 percent to 400 percent, 50 percent to 100 percent, 50 percent to 200 percent, 50 percent to 300 percent, 50 percent to 400 percent, 100 percent to 200 percent, 100 percent to 300 percent, 100 percent to 400 percent, 200 percent to 300 percent, 200 percent to 400 percent, or 300 percent to 400 percent higher than the mechanical stiffness of the gel substrate 120 of the patch device. In some cases, the mechanical stiffness of a backing component of a patch device is 10 percent, 50 percent, 100 percent, 200 percent, 300 percent, or 400 percent higher than the mechanical stiffness of the gel substrate 120 of the patch device. In some cases, the mechanical stiffness of a backing component of a patch device is at least 10 percent, 50 percent, 100 percent, 200 percent, 300 percent, or 400 percent higher than the mechanical stiffness of the gel substrate 120 of the patch device. In some cases, the mechanical stiffness of a backing component of a patch device is at most 50 percent, 100 percent, 200 percent, 300 percent, or 400 percent higher than the mechanical stiffness of the gel substrate 120 of the patch device.

Increasing the density of fibrin in a backing component of a patch device increases the mechanical stiffness of the backing component, in many cases. In some cases, fabricating a patch device having a backing component with a higher mechanical stiffness than the gel substrate to which it is attached allows the patch device to be manipulated more easily than a similar patch device lacking a backing component (e.g., by providing a surface that can be grasped firmly during application, for example, using forceps).

Patch devices disclosed herein comprise an adhesive, in many embodiments. An adhesive applied to a surface of a gel substrate (e.g., a surface (e.g., second surface 170) of gel substrate 120 in contact with a target area of a subject during treatment with the patch device) aids in holding the patch device in place relative to a target area of the subject in need of treating. In many cases, the adhesive is a fibrin glue or hyaluronic acid. In some cases, a force perpendicular to the surface of the target area is beneficial to the transfer of cells of the cellular component of a patch device to the target area. In some embodiments, a bandage (e.g., a Tegaderm® bandage) is placed over the patch device after the patch device is applied to the target area, for example, to apply or increase a force exerted on the target area by the patch device that is perpendicular to the target area's surface.

### I. Systems and Devices

Disclosed herein, in some embodiments, are devices and systems useful for treating skin depigmentation, skin hypopigmentation, and/or skin wounds in a subject. In some cases, the systems of the present disclosure are exemplified in FIG. 1A and FIG. 1B as an integrated device. In some cases, the device comprises a patch device that is adhesive to a surface, such as the surface of a subject's skin (e.g., a target area of the subject's skin in need of treating). The patch devices of the present disclosure comprise a gel substrate (e.g., hydrogel). In some cases, the gel substrate comprises a cellular component that comprises melanocytes that were purified and expanded ex vivo. The patch device comprises an adhesive that enables the patch device to adhere to a surface (e.g., a surface of the skin), in some embodiments.

A. Cellular Components

Disclosed herein, the systems and devices, such as those exemplified in FIG. 1A and FIG. 1B, comprise a cellular component 110. In some cases, including embodiments exemplified in FIG. 1A, patch device 100 comprises a cellular component 110. A cellular component 110 of patch device 100 comprises a plurality of cells, in many cases. In many cases, the identity and number of cells used to fabricate patch device 100 depends on the application for which patch device 100 is to be used. In some embodiments described herein, patch devices 100 comprising melanin-producing cells (e.g., melanocytes) are especially well-suited for applications in which matching skin pigmentation to a target location of a subject is intended, for example, in the treatment a skin condition characterized by hypopigmentation or depigmentation, such as vitiligo.

A patch device 100 disclosed herein comprises a cellular component 110, in many cases. In many embodiments, all or a portion of cellular component 110 of patch device 100 is transferred to a target area of a subject's skin, e.g., for the purpose of treating a skin condition such as hypopigmentation or depigmentation. For example, all or a portion of a population of primary melanocytes comprising cellular component 110 of patch device 100 is transferred to a target area of a subject's body in need of treating (e.g., a depigmented portion of the patient's skin) for the purpose of treating the target area, in some embodiments.

A cellular component 110 of a patch device 100 comprises a population of cells, in many embodiments. In many cases, a cellular component 110 comprises at least one cell. In many cases, a cellular component 110 comprises a plurality of cells. In some cases, cellular component 110 comprises no more than 1 type of cells. In some cases, cellular component 110 comprises two, three, four, five, six, seven, eight, nine, ten, or more than ten different types of cells. In some cases, the cellular component comprises at least two, at least three, at least four, at least five, at least six, at least seven, at least eight, at least nine, or at least ten different types of cells.

In many cases, cellular component 110 comprises at least one pigment-producing cell (e.g., melanocyte) or at least one precursor cell capable of giving rise to a pigment-producing cell (e.g., at least one pluripotent, multipotent, or unipotent precursor cell). In some cases, one or more pigment-producing cells (e.g., melanocytes) of cellular component 110 are derived from an organism (e.g., primary pigment-producing cells, such as primary melanocytes). In many cases, pigment-producing cells, including those derived from an organism, improve the repigmentation of a target area of a subject's body treated with patch device 100 (e.g., a subject's hypopigmented or depigmented skin tissue).

In some cases, cellular component 110 comprises one or more keratinocytes. In some cases, one or more keratinocyte of cellular component 110 are primary keratinocytes. In some cases, inclusion of at least one keratinocyte (e.g., a plurality of keratinocytes) in cellular component 110 improves barrier function of skin formed as a result of treatment with patch device 100. In some cases, the cellular component 110 comprises one or more fibroblasts.

In some cases, the cellular component comprises one or more cells selected from the group consisting of a melanocyte, a keratinocyte, and a fibroblast. In some cases, the cellular component comprise any two cells selected from the group consisting of melanocytes, keratinocytes, and fibroblasts. In some cases, the cellular component comprises melanocytes, keratinocytes, and fibroblasts. In some cases, all of the cells of patch device 100 are the same cell type. Patch devices 100 comprising cells that are all of the same cell type is easier to manufacture (e.g., fabricate) than patch devices 100 comprising multiple isolated cell types, in some cases, as derivation (e.g., isolation and/or differentiation) of additional cell types can increase cost and time associated with patch device manufacture. In some embodiments, the one or more cells in the cellular component are derived from a stem cell, such as a mesenchymal stem cell. Examples of stem cells include embryonic stem cells, induced pluripotent stem cells, bone marrow-derived stem cells, mesenchymal stem cells, and adipose-derived progenitor cells.

In some cases, cellular component 110 comprises a plurality of cells (e.g., primary melanocytes and/or primary keratinocytes) that have been purified and expanded ex vivo. In many cases, ex vivo expansion greatly increases the skin surface area that can be treated using one preparation of cells (e.g., one skin sample or one isolated population of primary melanocytes and/or primary keratinocytes isolated therefrom), for example, to treat skin hypopigmentation or depigmentation. In some cases, primary melanocytes from a relatively small pigmented portion of a subject's skin are expanded and used to treat a much larger area of depigmented skin. The ratio of the area of skin used to obtain melanocytes for use in the methods and devices herein to the area of skin treated with the expanded melanocytes is 1:5, in some cases. In some cases, the ratio of the area of skin used to obtain melanocytes to the area of skin treated with the expanded melanocytes is 1:1.1 to 1:125. In some cases, the ratio of the area of skin used to obtain melanocytes to the area of skin treated with the expanded melanocytes is 1:1.1 to 1:2, 1:1.1 to 1:3, 1:1.1 to 1:4, 1:1.1 to 1:5, 1:1.1 to 1:6, 1:1.1 to 1:7, 1:1.1 to 1:8, 1:1.1 to 1:9, 1:1.1 to 1:10, 1:1.1 to 1:20, 1:2 to 1:3, 1:2 to 1:4, 1:2 to 1:5, 1:2 to 1:6, 1:2 to 1:7, 1:2 to 1:8, 1:2 to 1:9, 1:2 to 1:10, 1:2 to 1:20, 1:3 to 1:4, 1:3 to 1:5, 1:3 to 1:6, 1:3 to 1:7, 1:3 to 1:8, 1:3 to 1:9, 1:3 to 1:10, 1:3 to 1:20, 1:4 to 1:5, 1:4 to 1:6, 1:4 to 1:7, 1:4 to 1:8, 1:4 to 1:9, 1:4 to 1:10, 1:4 to 1:20, 1:5 to 1:6, 1:5 to 1:7, 1:5 to 1:8, 1:5 to 1:9, 1:5 to 1:10, 1:5 to 1:20, 1:6 to 1:7, 1:6 to 1:8, 1:6 to 1:9, 1:6 to 1:10, 1:6 to 1:20, 1:7 to 1:8, 1:7 to 1:9, 1:7 to 1:10, 1:7 to 1:20, 1:8 to 1:9, 1:8 to 1:10, 1:8 to 1:20, 1:9 to 1:10, 1:9 to 1:20, 1:10 to 1:20, 1:1 to 1:25, 1:1 to 1:30, 1.1 to 1:40, 1:1 to 1:50, 1:1 to 1:60, 1:1 to 1:70, 1:1 to 1:80, 1:1 to 1:90, 1:1 to 1:100, 1:1 to 1:125, more than 1:1, more than 1:2, more than 1:3, more than 1:4, more than 1:5, more than 1:6, more than 1:7, more than 1:8, more than 1:9, more than 1:10, more than 1:15, more than 1:20, more than 1:15, more than 1:30, more than 1:35, more than 1:40, more than 1:50, more than 1:60, more than 1:70, more than 1:80, more than 1:90, more than 1:100, about 1:1, about 1:2, about 1:3, about 1:4, about 1:5, about 1:6, about 1:7, about 1:8, about 1:9, about 1:10, about 1:15, about 1:20, about 1:15, about 1:30, about 1:35, about 1:40, about 1:50, about 1:60, about 1:70, about 1:80, about 1:90, about 1:100, or greater than 1:20. In some cases, the ratio of the area of skin used to obtain melanocytes to the area of skin treated with the expanded melanocytes is 1:1.1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, or 1:100. In some cases, the ratio of the area of skin used to obtain melanocytes to the area of skin treated with the expanded melanocytes is at least 1:1.1, 1:2, 1:3, 1:4, 1:5, 1:6, 1:7, 1:8, 1:9, 1:10, 1:15, 1:20, or 1:25. In some cases, the ratio of the area of skin used to obtain melanocytes to the area of skin treated with the expanded melanocytes is at most 1:2, 1:3, 1:4, 1:5, 1:6, 1:7, 1:8, 1:9, 1:10, 1:10, 1:15, 1:20, 1:25, 1:50, 1:70, 1:80, 1:90, 1:100, or 1:125. In particular, the patch devices and methods disclosed herein are particularly useful in the treatment of vitiligo and other causes of hypopigmentation or depigmentation, including but not limited to scarred skin tissue.

In some embodiments, melanocytes cells comprise 50 percent to 99.5 percent of a cellular component 110 of a patch device 100. In some embodiments, melanocytes cells comprise 50 percent to 75 percent, 50 percent to 80 percent, 50 percent to 85 percent, 50 percent to 90 percent, 50 percent to 95 percent, 50 percent to 99 percent, 50 percent to 99.5 percent, 75 percent to 80 percent, 75 percent to 85 percent, 75 percent to 90 percent, 75 percent to 95 percent, 75 percent to 99 percent, 75 percent to 99.5 percent, 80 percent to 85 percent, 80 percent to 90 percent, 80 percent to 95 percent, 80 percent to 99 percent, 80 percent to 99.5 percent, 85 percent to 90 percent, 85 percent to 95 percent, 85 percent to 99 percent, 85 percent to 99.5 percent, 90 percent to 95 percent, 90 percent to 99 percent, 90 percent to 99.5 percent, 95 percent to 99 percent, 95 percent to 99.5 percent, or 99 percent to 99.5 percent. In some embodiments, melanocytes cells comprise 50 percent, 75 percent, 80 percent, 85 percent, 90 percent, 95 percent, 99 percent, or 99.5 percent of a cellular component 110 of a patch device 100. In some embodiments, melanocytes cells comprise at least 50 percent, 75 percent, 80 percent, 85 percent, 90 percent, 95 percent, or 99 percent of a cellular component 110 of a patch device 100. In some embodiments, melanocytes cells comprise at most 75 percent, 80 percent, 85 percent, 90 percent, 95 percent, 99 percent, or 99.5 percent of a cellular component 110 of a patch device 100. In some embodiments, human primary melanocytes cells comprise 50 percent to 99.5 percent of a cellular component 110 of a patch device 100. In some embodiments, human primary melanocytes cells comprise 50 percent to 75 percent, 50 percent to 80 percent, 50 percent to 85 percent, 50 percent to 90 percent, 50 percent to 95 percent, 50 percent to 99 percent, 50 percent to 99.5 percent, 75 percent to 80 percent, 75 percent to 85 percent, 75 percent to 90 percent, 75 percent to 95 percent, 75 percent to 99 percent, 75 percent to 99.5 percent, 80 percent to 85 percent, 80 percent to 90 percent, 80 percent to 95 percent, 80 percent to 99 percent, 80 percent to 99.5 percent, 85 percent to 90 percent, 85 percent to 95 percent, 85 percent to 99 percent, 85 percent to 99.5 percent, 90 percent to 95 percent, 90 percent to 99 percent, 90 percent to 99.5 percent, 95 percent to 99 percent, 95 percent to 99.5 percent, or 99 percent to 99.5 percent of a cellular component 110 of a patch device 100. In some embodiments, human primary melanocytes cells comprise 50 percent, 75 percent, 80 percent, 85 percent, 90 percent, 95 percent, 99 percent, or 99.5 percent of a cellular component 110 of a patch device 100. In some embodiments, human primary melanocytes cells comprise at least 50 percent, 75 percent, 80 percent, 85 percent, 90 percent, 95 percent, or 99 percent of a cellular component 110 of a patch device 100. In some embodiments, human primary melanocytes cells comprise at most 75 percent, 80 percent, 85 percent, 90 percent, 95 percent, 99 percent, or 99.5 percent of a cellular component 110 of a patch device 100.

In some cases, patch device 100 comprises more than one cell type (e.g., a plurality of cell types comprising a first cell type and a second cell type). For example, patch device 100 can comprise a melanocyte and a keratinocyte. In some cases, patch device 100 comprises 1, 2, 3, 4, 5, 6, 7, 8, 9, 10, 11, 12, 13, 14, 15, 16, 17, 18, 19, 20, or more than 20 different types of cells. The properties of patch device 100 are tunable based on the identities and ratios of cell types that it comprises. For example, increasing the ratio of keratinocytes to melanocytes in a patch device can increase the barrier function of an area of skin treated with the patch device. In some embodiments, the ratio of a first cell type of patch device 100 to a second cell type of patch device 100 is from 1:0.001 to 1:1,000. In some embodiments, the ratio of a first cell type of patch device 100 to a second cell type of patch device 100 is 1:1,000 to 1:100, 1:1,000 to 1:20, 1:1,000 to 1:10, 1:1,000 to 1:5, 1:1,000 to 1:2, 1:1,000 to 1:1, 1:1,000 to 1:0.5, 1:1,000 to 1:0.1, 1:1,000 to 1:0.05, 1:1,000 to 1:0.01, 1:1,000 to 1:0.001, 1:100 to 1:20, 1:100 to 1:10, 1:100 to 1:5, 1:100 to 1:2, 1:100 to 1:1, 1:100 to 1:0.5, 1:100 to 1:0.1, 1:100 to 1:0.05, 1:100 to 1:0.01, 1:100 to 1:0.001, 1:20 to 1:10, 1:20 to 1:5, 1:20 to 1:2, 1:20 to 1:1, 1:20 to 1:0.5, 1:20 to 1:0.1, 1:20 to 1:0.05, 1:20 to 1:0.01, 1:20 to 1:0.001, 1:10 to 1:5, 1:10 to 1:2, 1:10 to 1:1, 1:10 to 1:0.5, 1:10 to 1:0.1, 1:10 to 1:0.05, 1:10 to 1:0.01, 1:10 to 1:0.001, 1:5 to 1:2, 1:5 to 1:1, 1:5 to 1:0.5, 1:5 to 1:0.1, 1:5 to 1:0.05, 1:5 to 1:0.01, 1:5 to 1:0.001, 1:2 to 1:1, 1:2 to 1:0.5, 1:2 to 1:0.1, 1:2 to 1:0.05, 1:2 to 1:0.01, 1:2 to 1:0.001, 1:1 to 1:0.5, 1:1 to 1:0.1, 1:1 to 1:0.05, 1:1 to 1:0.01, 1:1 to 1:0.001, 1:0.5 to 1:0.1, 1:0.5 to 1:0.05, 1:0.5 to 1:0.01, 1:0.5 to 1:0.001, 1:0.1 to 1:0.05, 1:0.1 to 1:0.01, 1:0.1 to 1:0.001, 1:0.05 to 1:0.01, 1:0.05 to 1:0.001, or 1:0.01 to 1:0.001. In some embodiments, the ratio of a first cell type of patch device 100 to a second cell type of patch device 100 is 1:1,000, 1:100, 1:20, 1:10, 1:5, 1:2, 1:1, 1:0.5, 1:0.1, 1:0.05, 1:0.01, or 1:0.001. In some embodiments, the ratio of a first cell type of patch device 100 to a second cell type of patch device 100 is at least 1:1,000, 1:100, 1:20, 1:10, 1:5, 1:2, 1:1, 1:0.5, 1:0.1, 1:0.05, or 1:0.01. In some embodiments, the ratio of a first cell type of patch device 100 to a second cell type of patch device 100 is at most 1:100, 1:20, 1:10, 1:5, 1:2, 1:1, 1:0.5, 1:0.1, 1:0.05, 1:0.01, or 1:0.001. In some cases, a first cell type of patch device 100 is a melanocyte. In some cases, a second cell type of patch device 100 is a keratinocyte. In some cases, a first cell type of patch device 100 is a melanocyte and a second cell type of patch device 100 is a keratinocyte. For example, patch device 100 comprises a 1:10 ratio of melanocytes to keratinocytes in some cases. In some cases, patch device 100 comprises a fibroblast.

In some embodiments, the one or more cells in the cellular component are derived from a subject directly or indirectly. In some cases, the one or more cells are obtained directly by tissue biopsy using suitable methods. In some embodiments, the one or more cells are obtained from a subject in need of treatment with the systems or devices (e.g., a patch device) disclosed herein. In such cases, the one or more cells are “autologous.” In some embodiments, the one or more cells is obtained from a donor. In such cases, the one or more cells are “allogenic.”

The source of cells used in patch device 100 can affect the performance of the patch device. Cells used in patch device 100 are primary cells, in many cases. Allogeneic cells and/or syngeneic cells are used as a cellular component of patch device 100 in many embodiments. For example, human cells from a donor are used in a patch device 100 to be used for a subject, in many cases. In some cases, autologous cells are used as a cellular component of patch device 100. Patch devices 100 comprising allogeneic and/or autologous cells offer the best immunocompatibility (e.g., as a result of identical or substantially identical major histocompatibility complex (MHC)/human leukocyte antigen (HLA) match with the subject/recipient), in many cases. Patch devices 100 with good immunocompatibility for a given subject generally leads to better graft incorporation and procedure success. Xenogenic cells are used as a cellular component of patch device 100 in some cases. In some embodiments, autologous cells comprise 50 percent to 100 percent of the cellular component of a patch device 100. In some embodiments, autologous cells comprise 50 percent to 75 percent, 50 percent to 80 percent, 50 percent to 85 percent, 50 percent to 90 percent, 50 percent to 95 percent, 50 percent to 99 percent, 50 percent to 99.5 percent, 75 percent to 80 percent, 75 percent to 85 percent, 75 percent to 90 percent, 75 percent to 95 percent, 75 percent to 99 percent, 75 percent to 99.5 percent, 80 percent to 85 percent, 80 percent to 90 percent, 80 percent to 95 percent, 80 percent to 99 percent, 80 percent to 99.5 percent, 85 percent to 90 percent, 85 percent to 95 percent, 85 percent to 99 percent, 85 percent to 99.5 percent, 90 percent to 95 percent, 90 percent to 99 percent, 90 percent to 99.5 percent, 95 percent to 99 percent, 95 percent to 99.5 percent, or 99 percent to 99.5 percent of the cellular component of a patch device 100. In some embodiments, autologous cells comprise 50 percent, 75 percent, 80 percent, 85 percent, 90 percent, 95 percent, 99 percent, or 99.5 percent of the cellular component of a patch device 100. In some embodiments, autologous cells comprise at least 50 percent, 75 percent, 80 percent, 85 percent, 90 percent, 95 percent, or 99 percent of the cellular component of a patch device 100. In some embodiments, autologous cells comprise at most 75 percent, 80 percent, 85 percent, 90 percent, 95 percent, 99 percent, or 99.5 percent of the cellular component of a patch device 100.

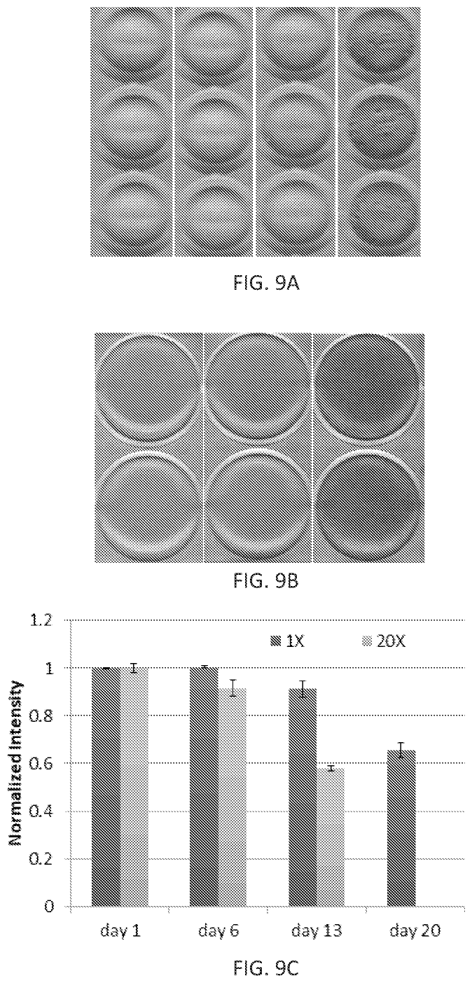

Fabrication of patch device 100 using primary cells of a higher passage allows coverage of a larger site, in many cases. In many cases, fabrication of patch device 100 using primary cells of a higher passage decreases the time required for pigmentation of a target site, in many cases. FIGS. 9A, 9B, and 9C show experimental data illustrating that higher passage melanocytes exhibit faster time to pigmentation, in some embodiments. For example, bars labeled “20×” in FIG. 9C show a population of melanocytes from the same cell line as the group labeled “1×” that has been expanded for 5 additional population doublings compared to its parent population show faster pigmentation (e.g., 13 days vs. 20 days), in some embodiments. For example, primary cells of passage number 1, 2, 3, 4, 5, 6, 7, 8, 9, or 10 are used in the fabrication of patch device 100, in many cases. Passage number can refer to the number of times a cell population has been subcultured (e.g., via enzymatic or mechanical removal from a culture surface and replating onto the same or a different culture surface). For example, primary cells that have been plated for the first time on a culture surface (e.g., after isolation from a biological source, such as a skin sample) are often referred to as passage 0 (e.g., p=0) cells. With each subsequent round of subculture, passage number increases by one integer. Melanocytes of a low passage useful in the fabrication of patch device 100 include cells of passages 0, 1, 2, 3, 4, 5, 6, 7, 8, 9, 10, 15, or 20. In some cases, at least a portion of the cells used in the fabrication of patch device 100 have been cultured for at least 1, at least 2, at least 3, at least 4, at least 5, at least 6, at least 7, at least 8, at least 9, at least 10, at least 15, or at least 20 passages. In some cases, a measure of population doublings is used to determine the age of a population of cells in culture. The number of population doublings per passage depends on the initial seeding density. Melanocytes having experienced 0, 1, 2, 3, 4, 5, 6, 7, 8, 9, 10, 11, 12, 13, 14, 15, 16, 17, 18, 19, or 20 population doublings (e.g., in culture) have strong viability and function when incorporated into patch device 100. In some cases, at least a portion of the cells (e.g., a plurality of pigment-producing cells, such as melanocytes) used in the fabrication of patch device 100 have experienced at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 population doublings in culture.

Cells derived from donors with healthy body mass index (BMI) values (e.g., from 18.5 to 25, from 18.5 to 24.9, from 19 to 24, from 20 to 23, or from 21 to 22) have good expansion potential, viability, and/or function, in many cases. In some cases, cells derived from donors who do not have (or have never had) type 2 diabetes can also offer good expansion potential, viability, and/or function. In some embodiments, allogenic cells are obtained from donors with healthy body mass index (BMI) values, such as BMI values from 18.5 to 25, from 18.5 to 24.9, from 19 to 24, from 20 to 23, or from 21 to 22, have good expansion potential, viability, and/or function. In some cases, autologous cells derived from donors who do not have (or have never had) type 2 diabetes also offer good expansion potential, viability, and/or function.

Cells to be used in the fabrication of patch device 100 are frozen (e.g., for long-term storage) prior to patch device fabrication, in some cases. For example, cells for use in patch device 100 can be frozen first at −80° C., and then transferred to liquid nitrogen. Freezing cells prior to use in the fabrication of a patch device 100 is useful in preserving expanded or unexpanded cells until needed. For example, receipt and/or isolation of primary cells (e.g., of a given MHC/HLA type) for use in fabrication of patch device 100 does not always coincide with demand for patch devices using those primary cells. Freezing (and subsequently thawing) cells to be used in patch device fabrication allows primary cell reagents to be used economically. In some cases, cells for use in patch device 100 are frozen using a freezing medium, for example, 10% serum (e.g., fetal bovine serum (FBS) or human serum), 10% dimethyl sulfoxide (DMSO), and 80% culture medium. In some cases, cells are frozen or otherwise preserved (e.g., stored at 4° C.) after incorporation into patch device 100.

In many embodiments, cells are not frozen prior to use in the fabrication of patch device 100. In some cases, using cells that have never been frozen improves the viability and/or function of cells used in patch device 100.

B. Gel Substrates

A patch device 100 comprises a gel substrate 120, in many cases (e.g., as shown in FIGS. 1A and 1B). A gel substrate 120 of a patch device 100 provides a substrate to which one or more cellular components (e.g., a plurality of cells comprising one or more cell types) of a patch device can adhere. A gel substrate 120 of a patch device 100 also provides mechanical structure and strength to the patch device, e.g., to maintain the patch device's shape after delivery and to maintain the position of cells of a patch device 100 prior to, during, and after application of the patch device 100 to a subject.

In many cases, gel substrate 120 comprises cellular component 110. For example, the cells comprising cellular component 110 are disposed within gel substrate 120, in many embodiments. Fabricating patch device 100 such that cells of cellular component 110 (e.g., cells to be transferred to a hypopigmented or depigmented target area of a subject during use of patch device 100) are within gel substrate 120 offers the advantage of controlling the distribution (e.g., spatial distribution) of the cells of cellular component 110 within patch device 100, in many cases. Control over the distribution of cells of cellular component 110 (e.g., pigment-producing cells, such as melanocytes) within gel substrate 120 allows control over the spatial distribution of the cells at a target area of a subject during and after treatment with patch device 100 (e.g., during and/or after contacting the target area with second surface 170 of gel substrate 120), in many cases. In many cases, control over the spatial distribution of cells (e.g., pigment-producing cells, such as melanocytes) at a target area of a subject during and after treatment with patch device 100 results in improved distribution (e.g., spatial distribution) of pigmentation at the target area. For example, pigment-producing cells can be maintained in an even distribution across an x-y plane 240 of gel substrate 120, resulting in even distribution of pigmentation in the target area after treatment, in many embodiments.

It is advantageous for a gel substrate 120 of a patch device 100 to be deformable. For example, a deformable gel substrate 120 allows the patch device 100 to conform to target tissue 140 (e.g., dermis of a subject being treated for vitiligo or scarring), which is advantageous in cases where the target tissue 140 is not substantially flat (e.g., a portion of a knee, elbow, or arm). In some cases, increasing the deformability of a patch device allows a greater percentage of the patch device (e.g., a greater percentage of the second surface 170 of patch device 100) to contact the target area of the subject. In some cases, increasing the percentage of the patch device in contact with the target area of the subject during treatment improves the efficiency with which cells are transferred from the patch device to the target area. In some cases, increasing the efficiency with which cells (e.g., cells of cellular component 110 of patch device 100) are transferred to from the patch device 100 to the target area increases the efficiency with which a skin condition, such as hypopigmentation or depigmentation, is treated. In conferring deformability on the patch device 100 while retaining sufficient mechanical structure and strength to maintain the structural integrity of the patch and to maintain the approximate positions of the cells comprising the cellular component 110 of patch device 100 within gel substrate 120, the gel substrate 120 allows the cells to be applied evenly to regions of a subject that are not flat in addition to regions of the subject that are flat.

The type(s) and/or ratio(s) of molecules comprising the gel substrate 120 affect the mechanical properties of the gel substrate 120, in many cases. In many cases, the type(s) and/or ratio(s) of molecules comprising the gel substrate 120 affects how the cells are distributed through the gel substrate.

A gel substrate 120 comprises at least one type of molecule. In some cases, a gel substrate 120 comprises a plurality of types of molecules. For example, a gel substrate 120 comprises 1, 2, 3, 4, 5, 6, 7, 8, 9, 10, 11, 12, 13, 14, 15, 16, 17, 18, 19, 20, or more than 20 types of molecules. In some cases, a gel substrate 120 comprises a matrix molecule. In some cases, a gel substrate 120 comprises a plurality of matrix molecules. For example, a gel substrate 120 comprises 1, 2, 3, 4, 5, 6, 7, 8, 9, 10, 11, 12, 13, 14, 15, 16, 17, 18, 19, 20, or more than 20 types of matrix molecules.

In many cases, a gel substrate 120 of a patch device 100 comprises fibrinogen. In some cases, the fibrinogen is bovine fibrinogen. In some cases, the fibrinogen is human fibrinogen. In many embodiments, a gel substrate of a patch device 100 comprises 10 mg/mL of fibrinogen. In some embodiments, the gel substrate comprises fibrinogen at a concentration of 1 mg/mL to 25 mg/mL. In some embodiments, the gel substrate comprises fibrinogen at a concentration of 1 mg/mL to 5 mg/mL, 1 mg/mL to 7.5 mg/mL, 1 mg/mL to 10 mg/mL, 1 mg/mL to 12.5 mg/mL, 1 mg/mL to 15 mg/mL, 1 mg/mL to 17.5 mg/mL, 1 mg/mL to 20 mg/mL, 1 mg/mL to 25 mg/mL, 5 mg/mL to 7.5 mg/mL, 5 mg/mL to 10 mg/mL, 5 mg/mL to 12.5 mg/mL, 5 mg/mL to 15 mg/mL, 5 mg/mL to 17.5 mg/mL, 5 mg/mL to 20 mg/mL, 5 mg/mL to 25 mg/mL, 7.5 mg/mL to 10 mg/mL, 7.5 mg/mL to 12.5 mg/mL, 7.5 mg/mL to 15 mg/mL, 7.5 mg/mL to 17.5 mg/mL, 7.5 mg/mL to 20 mg/mL, 7.5 mg/mL to 25 mg/mL, 10 mg/mL to 12.5 mg/mL, 10 mg/mL to 15 mg/mL, 10 mg/mL to 17.5 mg/mL, 10 mg/mL to 20 mg/mL, 10 mg/mL to 25 mg/mL, 12.5 mg/mL to 15 mg/mL, 12.5 mg/mL to 17.5 mg/mL, 12.5 mg/mL to 20 mg/mL, 12.5 mg/mL to 25 mg/mL, 15 mg/mL to 17.5 mg/mL, 15 mg/mL to 20 mg/mL, 15 mg/mL to 25 mg/mL, 17.5 mg/mL to 20 mg/mL, 17.5 mg/mL to 25 mg/mL, or 20 mg/mL to 25 mg/mL. In some embodiments, the gel substrate comprises fibrinogen at a concentration of 1 mg/mL, 5 mg/mL, 7.5 mg/mL, 10 mg/mL, 12.5 mg/mL, 15 mg/mL, 17.5 mg/mL, 20 mg/mL, or 25 mg/mL. In some embodiments, the gel substrate comprises fibrinogen at a concentration of at least 1 mg/mL, 5 mg/mL, 7.5 mg/mL, 10 mg/mL, 12.5 mg/mL, 15 mg/mL, 17.5 mg/mL, or 20 mg/mL. In some embodiments, the gel substrate comprises fibrinogen at a concentration of at most 5 mg/mL, 7.5 mg/mL, 10 mg/mL, 12.5 mg/mL, 15 mg/mL, 17.5 mg/mL, 20 mg/mL, or 25 mg/mL.

In many cases, a gel substrate 120 comprises fibrin. In some cases, the fibrin is bovine fibrin. In some cases, the fibrin is human fibrin. Higher concentrations of fibrin yield a more rigid gel substrate 120. In some embodiments, a more rigid gel substrate 120 (e.g., because of higher fibrin concentration) is advantageous because it is easier to manipulate before and during application of the patch device 100. In many embodiments, a gel substrate of a patch device 100 comprises 10 mg/mL of fibrin. In some embodiments, the gel substrate comprises fibrin at a concentration of 1 mg/mL to 17.5 mg/mL. In some embodiments, the gel substrate comprises fibrin at a concentration of 1 mg/mL to 5 mg/mL, 1 mg/mL to 7.5 mg/mL, 1 mg/mL to 10 mg/mL, 1 mg/mL to 12.5 mg/mL, 1 mg/mL to 15 mg/mL, 1 mg/mL to 17.5 mg/mL, 3 mg/mL to 4 mg/mL 5 mg/mL to 7.5 mg/mL, 5 mg/mL to 10 mg/mL, 5 mg/mL to 12.5 mg/mL, 5 mg/mL to 15 mg/mL, 5 mg/mL to 17.5 mg/mL, 7.5 mg/mL to 10 mg/mL, 7.5 mg/mL to 12.5 mg/mL, 7.5 mg/mL to 15 mg/mL, 7.5 mg/mL to 17.5 mg/mL, 10 mg/mL to 12.5 mg/mL, 10 mg/mL to 15 mg/mL, 10 mg/mL to 17.5 mg/mL, 10 mg/mL to 20 mg/mL, 10 mg/mL to 25 mg/mL, 12.5 mg/mL to 15 mg/mL, 12.5 mg/mL to 17.5 mg/mL, 12.5 mg/mL to 20 mg/mL, 12.5 mg/mL to 25 mg/mL, or 15 mg/mL to 17.5 mg/mL. In some embodiments, the gel substrate comprises fibrin at a concentration of 1 mg/mL, 2 mg/mL, 3 mg/mL, 4 mg/mL, 5 mg/mL, 7.5 mg/mL, 10 mg/mL, 12.5 mg/mL, 15 mg/mL, or 17.5 mg/mL. In some embodiments, the gel substrate comprises fibrin at a concentration of at least 1 mg/mL, 2 mg/mL, 3 mg/mL, 4 mg/mL, 5 mg/mL, 7.5 mg/mL, 10 mg/mL, 12.5 mg/mL, 15 mg/mL, or 17.5 mg/mL. In some embodiments, the gel substrate comprises fibrin at a concentration of at most 2 mg/mL, 3 mg/mL, 4 mg/mL, 5 mg/mL, 7.5 mg/mL, 10 mg/mL, 12.5 mg/mL, 15 mg/mL, or 17.5 mg/mL.

In many cases, a gel substrate 120 comprises thrombin. In some cases, the thrombin is bovine thrombin. In some cases, the thrombin is human thrombin. The concentration of thrombin many embodiments, a gel substrate of a patch device 100 comprises 2 U/mL of thrombin. In some embodiments, the gel substrate comprises thrombin at a concentration of 0.5 U/mL to 10 U/mL. In some embodiments, the gel substrate comprises thrombin at a concentration of 0.5 U/mL to 1 U/mL, 0.5 U/mL to 1.5 U/mL, 0.5 U/mL to 1.75 U/mL, 0.5 U/mL to 2 U/mL, 0.5 U/mL to 2.25 U/mL, 0.5 U/mL to 2.5 U/mL, 0.5 U/mL to 2.75 U/mL, 0.5 U/mL to 3 U/mL, 0.5 U/mL to 4 U/mL, 0.5 U/mL to 6 U/mL, 0.5 U/mL to 10 U/mL, 1 U/mL to 1.5 U/mL, 1 U/mL to 1.75 U/mL, 1 U/mL to 2 U/mL, 1 U/mL to 2.25 U/mL, 1 U/mL to 2.5 U/mL, 1 U/mL to 2.75 U/mL, 1 U/mL to 3 U/mL, 1 U/mL to 4 U/mL, 1 U/mL to 6 U/mL, 1 U/mL to 10 U/mL, 1.5 U/mL to 1.75 U/mL, 1.5 U/mL to 2 U/mL, 1.5 U/mL to 2.25 U/mL, 1.5 U/mL to 2.5 U/mL, 1.5 U/mL to 2.75 U/mL, 1.5 U/mL to 3 U/mL, 1.5 U/mL to 4 U/mL, 1.5 U/mL to 6 U/mL, 1.5 U/mL to 10 U/mL, 1.75 U/mL to 2 U/mL, 1.75 U/mL to 2.25 U/mL, 1.75 U/mL to 2.5 U/mL, 1.75 U/mL to 2.75 U/mL, 1.75 U/mL to 3 U/mL, 1.75 U/mL to 4 U/mL, 1.75 U/mL to 6 U/mL, 1.75 U/mL to 10 U/mL, 2 U/mL to 2.25 U/mL, 2 U/mL to 2.5 U/mL, 2 U/mL to 2.75 U/mL, 2 U/mL to 3 U/mL, 2 U/mL to 4 U/mL, 2 U/mL to 6 U/mL, 2 U/mL to 10 U/mL, 2.25 U/mL to 2.5 U/mL, 2.25 U/mL to 2.75 U/mL, 2.25 U/mL to 3 U/mL, 2.25 U/mL to 4 U/mL, 2.25 U/mL to 6 U/mL, 2.25 U/mL to 10 U/mL, 2.5 U/mL to 2.75 U/mL, 2.5 U/mL to 3 U/mL, 2.5 U/mL to 4 U/mL, 2.5 U/mL to 6 U/mL, 2.5 U/mL to 10 U/mL, 2.75 U/mL to 3 U/mL, 2.75 U/mL to 4 U/mL, 2.75 U/mL to 6 U/mL, 2.75 U/mL to 10 U/mL, 3 U/mL to 4 U/mL, 3 U/mL to 6 U/mL, 3 U/mL to 10 U/mL, 4 U/mL to 6 U/mL, 4 U/mL to 10 U/mL, or 6 U/mL to 10 U/mL. In some embodiments, the gel substrate comprises thrombin at a concentration of 0.5 U/mL, 1 U/mL, 1.5 U/mL, 1.75 U/mL, 2 U/mL, 2.25 U/mL, 2.5 U/mL, 2.75 U/mL, 3 U/mL, 4 U/mL, 6 U/mL, or 10 U/mL. In some embodiments, the gel substrate comprises thrombin at a concentration of at least 0.5 U/mL, 1 U/mL, 1.5 U/mL, 1.75 U/mL, 2 U/mL, 2.25 U/mL, 2.5 U/mL, 2.75 U/mL, 3 U/mL, 4 U/mL, or 6 U/mL. In some embodiments, the gel substrate comprises thrombin at a concentration of at most 1 U/mL, 1.5 U/mL, 1.75 U/mL, 2 U/mL, 2.25 U/mL, 2.5 U/mL, 2.75 U/mL, 3 U/mL, 4 U/mL, 6 U/mL, or 10 U/mL.

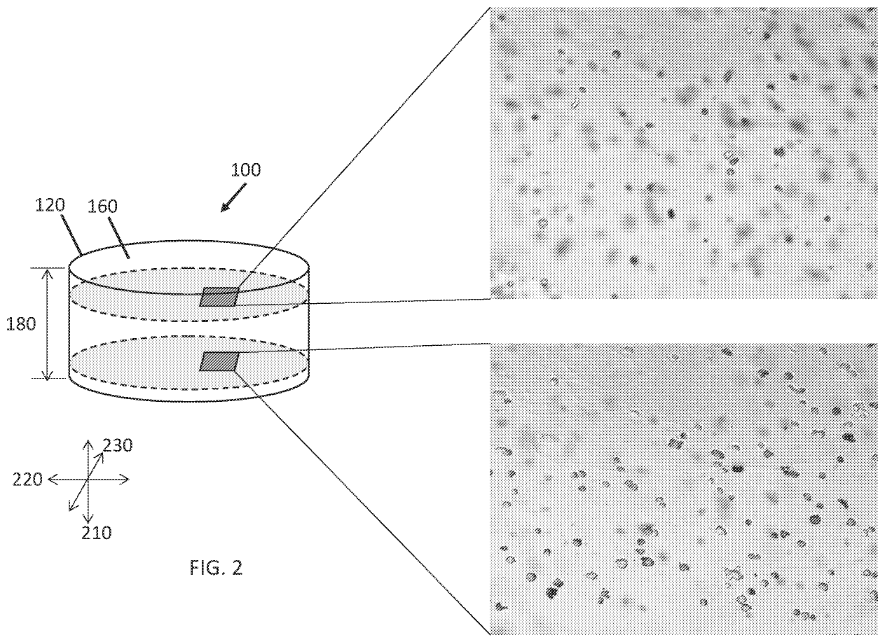

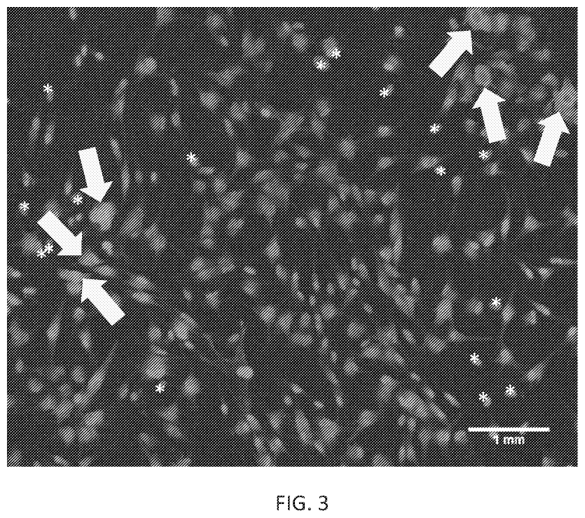

In many embodiments, the structure of a patch device 100 is controlled by the relative concentrations of fibrinogen and thrombin and/or the relative concentrations of fibrin and thrombin in gel substrate 120 during fabrication. Fibrinogen is converted to fibrin by the protease thrombin. The rate at which fibrinogen is converted to fibrin depends on the concentration of thrombin to which the fibrinogen is exposed. Because the initial concentration of fibrinogen in the gel substrate 120 impacts the amount of fibrin present in the final gel substrate 120 (e.g., after the fibrinogen is exposed to thrombin) and because fibrin provides mechanical strength to the gel substrate, the initial concentration of fibrinogen is directly related to the concentration of fibrin present in a gel substrate 120 that also comprises thrombin, in some embodiments. Furthermore, because the relative concentration of thrombin to the concentration of fibrinogen used in the fabrication of gel substrate 120 determines the rate at which fibrinogen is converted to fibrin, the ratio of fibrinogen to thrombin affects the structure of the patch device 100. For example, in cases where cells are added to the gel substrate during gel substrate fabrication, the rate at which fibrinogen is converted to fibrin (e.g., via exposure to thrombin) impacts the distribution of cells throughout the gel substrate 120 of the patch device 100, in many cases. In many embodiments, it is advantageous to form a gel substrate 120 such that the cells of the patch device are closest to the surface of the gel substrate 120 that will be applied to the target tissue 140 (e.g., the second surface 170 as shown in FIG. 1A and FIG. 1B). For example, transfer of cells comprising the cellular component 110 of patch device 100 is efficient when the cells are located near the surface of the patch device that will be applied to the target tissue 140 (e.g., second surface 170), in many embodiments. Thus, it is advantageous in many cases to add fibrinogen and thrombin during gel substrate fabrication in a ratio that allows the gel substrate 120 to completely gel (e.g., via interaction of thrombin with fibrinogen equally throughout the gel substrate during fabrication) while regulating the speed of gelation so that the cells settle to the bottom of the gel during gel substrate 120 fabrication (see, e.g., FIG. 2 showing bright field images in which the majority of cells are in focus when the microscope objective is focused on the bottom of the gel substrate and few cells are in focus when the microscope objective is focused in the middle of the gel). This is accomplished, for example, when the concentration of fibrinogen is from 10 mg/mL to 15 mg/mL and the concentration of thrombin is 2 U/mL. In some cases, the concentration of fibrinogen is from 10 mg/mL to 15 mg/mL and the concentration of thrombin is 4 U/mL. In some cases, the concentration of fibrinogen is from 10 mg/mL to 15 mg/mL, and the concentration of thrombin is 1 U/mL to 8 U/mL.

In many cases, a gel substrate 120 of a patch device 100 comprises fibrin and thrombin. In many cases, a gel substrate 120 comprises 10 mg/mL of fibrin and 2 U/mL of thrombin. In some cases, a gel substrate 120 comprises 15 mg/mL of fibrin and 2 U/mL of thrombin. In some embodiments, the gel substrate comprises 2 U/mL of thrombin and a fibrin concentration of 1 mg/mL to 17.5 mg/mL. In some embodiments, the gel substrate comprises 2 U/mL of thrombin and a fibrin concentration of 1 mg/mL to 5 mg/mL, 3 mg/mL to 4 mg/mL, 1 mg/mL to 7.5 mg/mL, 1 mg/mL to 10 mg/mL, 1 mg/mL to 12.5 mg/mL, 1 mg/mL to 15 mg/mL, 1 mg/mL to 17.5 mg/mL, 5 mg/mL to 7.5 mg/mL, 5 mg/mL to 10 mg/mL, 5 mg/mL to 12.5 mg/mL, 5 mg/mL to 15 mg/mL, 5 mg/mL to 17.5 mg/mL, 7.5 mg/mL to 10 mg/mL, 7.5 mg/mL to 12.5 mg/mL, 7.5 mg/mL to 15 mg/mL, 7.5 mg/mL to 17.5 mg/mL, 10 mg/mL to 12.5 mg/mL, 10 mg/mL to 15 mg/mL, 10 mg/mL to 17.5 mg/mL, 12.5 mg/mL to 15 mg/mL, 12.5 mg/mL to 17.5 mg/mL, or 15 mg/mL to 17.5 mg/mL. In some embodiments, the gel substrate comprises 2 U/mL of thrombin and a fibrin concentration of 1 mg/mL, 3 mg/mL, 4 mg/mL, 5 mg/mL, 7.5 mg/mL, 10 mg/mL, 12.5 mg/mL, 15 mg/mL, or 17.5 mg/mL. In some embodiments, the gel substrate comprises 2 U/mL of thrombin and a fibrin concentration of at least 1 mg/mL, 5 mg/mL, 7.5 mg/mL, 10 mg/mL, 12.5 mg/mL, 15 mg/mL, or 17.5 mg/mL. In some embodiments, the gel substrate comprises 2 U/mL of thrombin and a fibrin concentration of at most 5 mg/mL, 7.5 mg/mL, 10 mg/mL, 12.5 mg/mL, 15 mg/mL, or 17.5 mg/mL.

In many cases, a gel substrate 120 of a patch device 100 comprises fibrinogen and thrombin. In many cases, a gel substrate 120 comprises 10 mg/mL of fibrinogen and 2 U/mL of thrombin. In some cases, a gel substrate 120 comprises 15 mg/mL of fibrinogen and 2 U/mL of thrombin. In some embodiments, the gel substrate comprises 2 U/mL of thrombin and a fibrinogen concentration of 1 mg/mL to 17.5 mg/mL. In some embodiments, the gel substrate comprises 2 U/mL of thrombin and a fibrinogen concentration of 1 mg/mL to 5 mg/mL, 1 mg/mL to 7.5 mg/mL, 1 mg/mL to 10 mg/mL, 1 mg/mL to 12.5 mg/mL, 1 mg/mL to 15 mg/mL, 1 mg/mL to 17.5 mg/mL, 5 mg/mL to 7.5 mg/mL, 5 mg/mL to 10 mg/mL, 5 mg/mL to 12.5 mg/mL, 5 mg/mL to 15 mg/mL, 5 mg/mL to 17.5 mg/mL, 7.5 mg/mL to 10 mg/mL, 7.5 mg/mL to 12.5 mg/mL, 7.5 mg/mL to 15 mg/mL, 7.5 mg/mL to 17.5 mg/mL, 10 mg/mL to 12.5 mg/mL, 10 mg/mL to 15 mg/mL, 10 mg/mL to 17.5 mg/mL, 12.5 mg/mL to 15 mg/mL, 12.5 mg/mL to 17.5 mg/mL, 12.5 mg/mL to 20 mg/mL, 12.5 mg/mL to 25 mg/mL, or 15 mg/mL to 17.5 mg/mL. In some embodiments, the gel substrate comprises 2 U/mL of thrombin and a fibrinogen concentration of 1 mg/mL, 5 mg/mL, 7.5 mg/mL, 10 mg/mL, 12.5 mg/mL, 15 mg/mL, or 17.5 mg/mL. In some embodiments, the gel substrate comprises 2 U/mL of thrombin and a fibrinogen concentration of at least 1 mg/mL, 5 mg/mL, 7.5 mg/mL, 10 mg/mL, 12.5 mg/mL, 15 mg/mL, or 17.5 mg/mL. In some embodiments, the gel substrate comprises 2 U/mL of thrombin and a fibrinogen concentration of at most 5 mg/mL, 7.5 mg/mL, 10 mg/mL, 12.5 mg/mL, 15 mg/mL, or 17.5 mg/mL.

In many cases, a cellular component 110 is added to a gel substrate 120 during fabrication of the gel substrate. In some embodiments, the gel substrate comprises a first cell type at a concentration of 25,000 cells/cm2 to 150,000 cells/cm2. In some embodiments, the gel substrate comprises a first cell type at a concentration of 25,000 cells/cm2 to 50,000 cells/cm2, 25,000 cells/cm2 to 75,000 cells/cm2, 25,000 cells/cm2 to 100,000 cells/cm2, 25,000 cells/cm2 to 200,000 cells/cm2, 25,000 cells/cm2 to 300,000 cells/cm2, 25,000 cells/cm2 to 325,000 cells/cm2, 25,000 cells/cm2 to 150,000 cells/cm2, 50,000 cells/cm2 to 75,000 cells/cm2, 50,000 cells/cm2 to 100,000 cells/cm2, 50,000 cells/cm2 to 200,000 cells/cm2, 50,000 cells/cm2 to 300,000 cells/cm2, 50,000 cells/cm2 to 325,000 cells/cm2, 50,000 cells/cm2 to 150,000 cells/cm2, 75,000 cells/cm2 to 100,000 cells/cm2, 75,000 cells/cm2 to 200,000 cells/cm2, 75,000 cells/cm2 to 300,000 cells/cm2, 75,000 cells/cm2 to 325,000 cells/cm2, 75,000 cells/cm2 to 150,000 cells/cm2, 100,000 cells/cm2 to 325,000 cells/cm2, 100,000 cells/cm2 to 150,000 cells/cm2, 100,000 cells/cm2 to 200,000 cells/cm2, 100,000 cells/cm2 to 300,000 cells/cm2, or 325,000 cells/cm2 to 150,000 cells/cm2. In some embodiments, the gel substrate comprises a first cell type at a concentration of 25,000 cells/cm2, 50,000 cells/cm2, 75,000 cells/cm2, 100,000 cells/cm2, 125,000 cells/cm2, 200,000 cells/cm2, 250,000 cells/cm2, 300,000 cells/cm2, 325,000 cells/cm2, or 150,000 cells/cm2. In some embodiments, the gel substrate comprises a first cell type at a concentration of at least 25,000 cells/cm2, 50,000 cells/cm2, 75,000 cells/cm2, 100,000 cells/cm2, 125,000 cells/cm2, 200,000 cells/cm2, 250,000 cells/cm2, 300,000 cells/cm2, or 325,000 cells/cm2. In some embodiments, the gel substrate comprises a first cell type at a concentration of at most 50,000 cells/cm2, 75,000 cells/cm2, 100,000 cells/cm2, 125,000 cells/cm2, 200,000 cells/cm2, 250,000 cells/cm2, 300,000 cells/cm2, 325,000 cells/cm2, or 150,000 cells/cm2.

In some cases, all or a portion of the cellular component 110 (e.g., one or more of the cells comprising the patch device 100) are added to the reagents used to form the gel substrate 120 before or during formation (e.g., gelation) of gel substrate 120. For example, one or more cells of the patch device 100 are added to the fibrinogen and/or thrombin used to form the gel substrate 120 prior to the gelation of the gel substrate. In some cases, all or a portion of the cellular component 110 (e.g., one or more of the cells comprising the patch device 100) are added to the gel substrate 120 after the gel substrate 120 has congealed. For example, a solution of cells is seeded onto a surface of the gel substrate (e.g., a first surface and/or a second surface) by applying a volume of the solution of cells to the surface(s) of the gel substrate in droplets or aliquots, in some embodiments. In some cases, the congealed gel substrate 120 is fully or partially submerged in a solution of cells to be added to the patch device 100 to seed the gel substrate with cells.

The rate of forming a gel substrate 120 determines the distribution (e.g., spatial distribution) of cells of the cellular component 110 within the gel substrate 120, in some cases. The rate in which a gel substrate 120 is formed is controlled by changing one or more of the following parameters: (a) the amount of thrombin and/or the amount (e.g., concentration) of fibrinogen reacted to form the gel substrate; (b) the ratio of thrombin to fibrinogen in the reaction used to form the gel substrate; (c) the temperature at which the thrombin and the fibrinogen are reacted to form the gel substrate; and (d) the surface area-to-volume ratio of the gel substrate. For example, methods for forming patch devices wherein 50% to 100% of the cells of the cellular component of the patch device is located within 50% of the overall height of the device from a surface (e.g., second surface 170) of gel substrate 120 that is used to contact a target area of a subject during treatment.

In some embodiments, at least 50% of the cells of the cellular component 110 are disposed within a distance 190 of a second surface 170 of the gel substrate that is 5 percent to 50 percent of a height 180 of the gel substrate. In some embodiments, at least 50% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, at least 50% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, at least 50% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, at least 50% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments, at least 75% of the cells of the cellular component 110 are disposed within a distance 190 of a second surface 170 of the gel substrate that is 5 percent to 50 percent of a height 180 of the gel substrate. In some embodiments, at least 75% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, at least 75% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, at least 75% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, at least 75% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments, at least 80% of the cells of the cellular component 110 arm disposed within a distance 190 of a second surface 170 of the gel substrate that is 5 percent to 50 percent of a height 180 of the gel substrate. In some embodiments, at least 80% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, at least 80% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, at least 80% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, at least 80% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments, at least 90% of the cells of the cellular component 110 are disposed within a distance 190 of a second surface 170 of the gel substrate that is 5 percent to 50 percent of a height 180 of the gel substrate. In some embodiments, at least 90% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent. 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, at least 90% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, at least 90% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, at least 90% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments, at least 95% of the cells of the cellular component 110 are disposed within a distance 190 of a second surface 170 of the gel substrate that is 5 percent to 50 percent of a height 180 of the gel substrate. In some embodiments, at least 95% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, at least 95% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, at least 95% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, at least 95% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments. 100% of the cells of the cellular component 110 are disposed within a distance 190 of a second surface 170 of the gel substrate that is 5 percent to 50 percent of a height 180 of the gel substrate. In some embodiments, 100% of the cells of the cellular component 110 are disposed within a distance 190 of a second surface 170 of the gel substrate that is 50 percent to 45 percent, 50 percent to 40 percent, 50 percent to 35 percent, 50 percent to 30 percent, 50 percent to 25 percent, 50 percent to 20 percent, 50 percent to 15 percent, 50 percent to 10 percent, 50 percent to 5 percent, 45 percent to 40 percent, 45 percent to 35 percent, 45 percent to 30 percent, 45 percent to 25 percent, 45 percent to 20 percent, 45 percent to 15 percent, 45 percent to 10 percent, 45 percent to 5 percent, 40 percent to 35 percent, 40 percent to 30 percent, 40 percent to 25 percent, 40 percent to 20 percent, 40 percent to 15 percent, 40 percent to 10 percent, 40 percent to 5 percent, 35 percent to 30 percent, 35 percent to 25 percent, 35 percent to 20 percent, 35 percent to 15 percent, 35 percent to 10 percent, 35 percent to 5 percent, 30 percent to 25 percent, 30 percent to 20 percent, 30 percent to 15 percent, 30 percent to 10 percent, 30 percent to 5 percent, 25 percent to 20 percent, 25 percent to 15 percent, 25 percent to 10 percent, 25 percent to 5 percent, 20 percent to 15 percent, 20 percent to 10 percent, 20 percent to 5 percent, 15 percent to 10 percent, 15 percent to 5 percent, or 10 percent to 5 percent of a height 180 of the gel substrate. In some embodiments, 100% of the cells of the cellular component 110 are disposed within a distance 190 of a second surface 170 of the gel substrate that is 50 percent, 45 percent, 40 percent, 35 percent, 30 percent, 25 percent, 20 percent, 15 percent, 10 percent, or 5 percent of a height 180 of the gel substrate. In some embodiments, 100% of the cells of the cellular component 110 are disposed within a distance 190 of a second surface 170 of the gel substrate that is at least 50 percent, 45 percent, 40 percent, 35 percent, 30 percent, 25 percent, 20 percent, 15 percent, or 10 percent of a height 180 of the gel substrate. In some embodiments, 100% of the cells of the cellular component 110 are disposed within a distance 190 of a second surface 170 of the gel substrate that is at most 50 percent, at most 45 percent, 40 percent, 35 percent, 30 percent, 25 percent, 20 percent, 15 percent, 10 percent, or 5 percent of a height 180 of the gel substrate.

In some embodiments, the gel substrate 120 has a height 180 of 1 millimeter to 10 millimeters. In some embodiments, the gel substrate 120 has a height 180 of 1 millimeter to 2 millimeters, 1 millimeter to 3 millimeters, 1 millimeter to 4 millimeters, 1 millimeter to 5 millimeters, 1 millimeter to 10 millimeters, 2 millimeters to 3 millimeters, 2 millimeters to 4 millimeters, 2 millimeters to 5 millimeters, 2 millimeters to 10 millimeters, 3 millimeters to 4 millimeters, 3 millimeters to 5 millimeters, 3 millimeters to 10 millimeters, 4 millimeters to 5 millimeters, 4 millimeters to 10 millimeters, or 5 millimeters to 10 millimeters. In some embodiments, the gel substrate 120 has a height 180 of 1 millimeter, 2 millimeters, 3 millimeters, 4 millimeters, 5 millimeters, or 10 millimeters. In some embodiments, the gel substrate 120 has a height 180 of at least 1 millimeter, 2 millimeters, 3 millimeters, 4 millimeters, or 5 millimeters. In some embodiments, the gel substrate 120 has a height 180 of at most 2 millimeters, 3 millimeters, 4 millimeters, 5 millimeters, or 10 millimeters.

In some embodiments, the gel substrate 120 has a width (e.g., diameter or maximum cross-sectional dimension) of 1 millimeter to 50 millimeters. In some embodiments, the gel substrate 120 has a width (e.g., diameter or maximum cross-sectional dimension) of 1 millimeter to 2.5 millimeters, 1 millimeter to 5 millimeters, 1 millimeter to 7.5 millimeters, 1 millimeter to 10 millimeters, 1 millimeter to 15 millimeters, 1 millimeter to 20 millimeters, 1 millimeter to 50 millimeters, 2.5 millimeters to 5 millimeters, 2.5 millimeters to 7.5 millimeters, 2.5 millimeters to 10 millimeters, 2.5 millimeters to 15 millimeters, 2.5 millimeters to 20 millimeters, 2.5 millimeters to 50 millimeters, 5 millimeters to 7.5 millimeters, 5 millimeters to 10 millimeters, 5 millimeters to 15 millimeters, 5 millimeters to 20 millimeters, 5 millimeters to 50 millimeters, 7.5 millimeters to 10 millimeters, 7.5 millimeters to 15 millimeters, 7.5 millimeters to 20 millimeters, 7.5 millimeters to 50 millimeters, 10 millimeters to 15 millimeters, 10 millimeters to 20 millimeters, 10 millimeters to 50 millimeters, 15 millimeters to 20 millimeters, 15 millimeters to 50 millimeters, or 20 millimeters to 50 millimeters. In some embodiments, the gel substrate 120 has a width (e.g., diameter or maximum cross-sectional dimension) of 1 millimeter, 2.5 millimeters, 5 millimeters, 7.5 millimeters, 10 millimeters, 15 millimeters, 20 millimeters, or 50 millimeters. In some embodiments, the gel substrate 120 has a width (e.g., diameter or maximum cross-sectional dimension) of at least 1 millimeter, 2.5 millimeters, 5 millimeters, 7.5 millimeters, 10 millimeters, 15 millimeters, 20 millimeters, or 50 millimeters. In some embodiments, the gel substrate 120 has a width (e.g., diameter or maximum cross-sectional dimension) of at most 2.5 millimeters, 5 millimeters, 7.5 millimeters, 10 millimeters, 15 millimeters, 20 millimeters, or 50 millimeters.

In some embodiments, the gel substrate 120 has a length of 1 millimeter to 50 millimeters. In some embodiments, the gel substrate 120 has a length of 1 millimeter to 2.5 millimeters, 1 millimeter to 5 millimeters, 1 millimeter to 7.5 millimeters, 1 millimeter to 10 millimeters, 1 millimeter to 15 millimeters, 1 millimeter to 20 millimeters, 1 millimeter to 50 millimeters, 2.5 millimeters to 5 millimeters, 2.5 millimeters to 7.5 millimeters, 2.5 millimeters to 10 millimeters, 2.5 millimeters to 15 millimeters, 2.5 millimeters to 20 millimeters, 2.5 millimeters to 50 millimeters, 5 millimeters to 7.5 millimeters, 5 millimeters to 10 millimeters, 5 millimeters to 15 millimeters, 5 millimeters to 20 millimeters, 5 millimeters to 50 millimeters, 7.5 millimeters to 10 millimeters, 7.5 millimeters to 15 millimeters, 7.5 millimeters to 20 millimeters, 7.5 millimeters to 50 millimeters, 10 millimeters to 15 millimeters, 10 millimeters to 20 millimeters, 10 millimeters to 50 millimeters, 15 millimeters to 20 millimeters, 15 millimeters to 50 millimeters, or 20 millimeters to 50 millimeters. In some embodiments, the gel substrate 120 has a length of 1 millimeter, 2.5 millimeters, 5 millimeters, 7.5 millimeters, 10 millimeters, 15 millimeters, 20 millimeters, or 50 millimeters. In some embodiments, the gel substrate 120 has a length of at least 1 millimeter, 2.5 millimeters, 5 millimeters, 7.5 millimeters, 10 millimeters, 15 millimeters, 20 millimeters, or 50 millimeters. In some embodiments, the gel substrate 120 has a length of at most 2.5 millimeters, 5 millimeters, 7.5 millimeters, 10 millimeters, 15 millimeters, 20 millimeters, or 50 millimeters.

In many cases, patch device 100 or portion thereof (e.g., a non-cellular component of the patch device, such as gel substrate 120, adhesive 150, and/or backing component 130) degrades (e.g., partially or completely) during use. For example, patch device 100 or a portion thereof (e.g., gel substrate 120, adhesive 150, and/or backing component 130) degrades after being applied to the target area that is being treated with the patch device, in many embodiments. In many cases, patch device 100 or a portion thereof (e.g., gel substrate 120, adhesive 150, and/or backing component 130) degrades, completely or partially, while in contact with a target area to which it is applied (e.g., during treatment of a subject). In many cases, patch device 100 or portion thereof is substantially biodegradable while in contact with a target area to which it is applied (e.g., during treatment of a subject). In many cases, degradation of one or more components of patch device 100 (e.g., gel substrate 120, adhesive 150, and/or backing component 130) improves the ease of use of the patch device. For example, a patch device that degrades partially or completely during use reduces or eliminates the need for a practitioner to remove undegraded portions of the patch device from the subject's body after application during treatment (e.g., as compared to treatment with a device comprising non-degradable components). In some cases, reduction or elimination of the need for a practitioner to remove undegraded portions of the patch device from a subject's body after application during a treatment reduces the risk of infection or inflammation, for example because fewer interventions, which may stimulate inflammation or lead to infection, are needed at the treatment site.

In some cases, lower concentrations of fibrin in the gel substrate 120 result in faster degradation of the gel substrate 120 after application of the patch device 100 to a treatment area (e.g., target tissue 140 of a subject). Faster degradation of a gel substrate 120 that retains the benefits of fibrin is advantageous in many cases because it reduces the time that a gel substrate 120 is present on the target tissue 140 while maintaining spatial positioning of the cellular component of the patch device 100. In some cases, a gel substrate 120 degrades in less than 3 days (e.g., after application to a surface of a subject's body). In some embodiments, a gel substrate 120 degrades in 1 hour to 168 hours. In some embodiments, a gel substrate 120 degrades in 1 hour to 12 hours, 1 hour to 24 hours, 1 hour to 36 hours, 1 hour to 42 hours, 1 hour to 48 hours, 1 hour to 54 hours, 1 hour to 60 hours, 1 hour to 72 hours, 1 hour to 96 hours, 1 hour to 120 hours, 1 hour to 168 hours, 12 hours to 24 hours, 12 hours to 36 hours, 12 hours to 42 hours, 12 hours to 48 hours, 12 hours to 54 hours, 12 hours to 60 hours, 12 hours to 72 hours, 12 hours to 96 hours, 12 hours to 120 hours, 12 hours to 168 hours, 24 hours to 36 hours, 24 hours to 42 hours, 24 hours to 48 hours, 24 hours to 54 hours, 24 hours to 60 hours, 24 hours to 72 hours, 24 hours to 96 hours, 24 hours to 120 hours, 24 hours to 168 hours, 36 hours to 42 hours, 36 hours to 48 hours, 36 hours to 54 hours, 36 hours to 60 hours, 36 hours to 72 hours, 36 hours to 96 hours, 36 hours to 120 hours, 36 hours to 168 hours, 42 hours to 48 hours, 42 hours to 54 hours, 42 hours to 60 hours, 42 hours to 72 hours, 42 hours to 96 hours, 42 hours to 120 hours, 42 hours to 168 hours, 48 hours to 54 hours, 48 hours to 60 hours, 48 hours to 72 hours, 48 hours to 96 hours, 48 hours to 120 hours, 48 hours to 168 hours, 54 hours to 60 hours, 54 hours to 72 hours, 54 hours to 96 hours, 54 hours to 120 hours, 54 hours to 168 hours, 60 hours to 72 hours, 60 hours to 96 hours, 60 hours to 120 hours, 60 hours to 168 hours, 72 hours to 96 hours, 72 hours to 120 hours, 72 hours to 168 hours, 96 hours to 120 hours, 96 hours to 168 hours, or 120 hours to 168 hours. In some embodiments, a gel substrate 120 degrades in 1 hour, 12 hours, 24 hours, 36 hours, 42 hours, 48 hours, 54 hours, 60 hours, 72 hours, 96 hours, 120 hours, or 168 hours. In some embodiments, a gel substrate 120 degrades in at least 1 hour, 12 hours, 24 hours, 36 hours, 42 hours, 48 hours, 54 hours, 60 hours, 72 hours, 96 hours, or 120 hours. In some embodiments, a gel substrate 120 degrades in at most 12 hours, 24 hours, 36 hours, 42 hours, 48 hours, 54 hours, 60 hours, 72 hours, 96 hours, 120 hours, or 168 hours.

In most cases, at least a portion of the cells comprising cellular component 110 do not degrade during use of patch device 100. In some cases, 20 percent to 100 percent do not degrade during use of patch device 100. In some cases, 20 percent to 40 percent, 20 percent to 50 percent, 20 percent to 60 percent, 20 percent to 70 percent, 20 percent to 80 percent, 20 percent to 85 percent, 20 percent to 90 percent, 20 percent to 95 percent, 20 percent to 100 percent, 40 percent to 50 percent, 40 percent to 60 percent, 40 percent to 70 percent, 40 percent to 80 percent, 40 percent to 85 percent, 40 percent to 90 percent, 40 percent to 95 percent, 40 percent to 100 percent, 50 percent to 60 percent, 50 percent to 70 percent, 50 percent to 80 percent, 50 percent to 85 percent, 50 percent to 90 percent, 50 percent to 95 percent, 50 percent to 100 percent, 60 percent to 70 percent, 60 percent to 80 percent, 60 percent to 85 percent, 60 percent to 90 percent, 60 percent to 95 percent, 60 percent to 100 percent, 70 percent to 80 percent, 70 percent to 85 percent, 70 percent to 90 percent, 70 percent to 95 percent, 70 percent to 100 percent, 80 percent to 85 percent, 80 percent to 90 percent, 80 percent to 95 percent, 80 percent to 100 percent, 85 percent to 90 percent, 85 percent to 95 percent, 85 percent to 100 percent, 90 percent to 95 percent, 90 percent to 100 percent, or 95 percent to 100 percent do not degrade during use of patch device 100. In some cases, 20 percent, 40 percent, 50 percent, 60 percent, 70 percent, 80 percent, 85 percent, 90 percent, 95 percent, or 100 percent do not degrade during use of patch device 100. In some cases, at least 20 percent, 40 percent, 50 percent, 60 percent, 70 percent, 80 percent, 85 percent, 90 percent, or 95 percent do not degrade during use of patch device 100. In some cases, at most 40 percent, 50 percent, 60 percent, 70 percent, 80 percent, 85 percent, 90 percent, 95 percent, or 100 percent do not degrade during use of patch device 100.

In many cases, at least a portion of the cells comprising cellular component 110 integrate into a host tissue of the subject (e.g., a host tissue in or adjacent to a target area treated with the patch device). In some cases, 20 percent to 100 percent integrate into a host tissue of the subject (e.g., a host tissue in or adjacent to a target area treated with the patch device). In some cases, 20 percent to 40 percent, 20 percent to 50 percent, 20 percent to 60 percent, 20 percent to 70 percent, 20 percent to 80 percent, 20 percent to 85 percent, 20 percent to 90 percent, 20 percent to 95 percent, 20 percent to 100 percent, 40 percent to 50 percent, 40 percent to 60 percent, 40 percent to 70 percent, 40 percent to 80 percent, 40 percent to 85 percent, 40 percent to 90 percent, 40 percent to 95 percent, 40 percent to 100 percent, 50 percent to 60 percent, 50 percent to 70 percent, 50 percent to 80 percent, 50 percent to 85 percent, 50 percent to 90 percent, 50 percent to 95 percent, 50 percent to 100 percent, 60 percent to 70 percent, 60 percent to 80 percent, 60 percent to 85 percent, 60 percent to 90 percent, 60 percent to 95 percent, 60 percent to 100 percent, 70 percent to 80 percent, 70 percent to 85 percent, 70 percent to 90 percent, 70 percent to 95 percent, 70 percent to 100 percent, 80 percent to 85 percent, 80 percent to 90 percent, 80 percent to 95 percent, 80 percent to 100 percent, 85 percent to 90 percent, 85 percent to 95 percent, 85 percent to 100 percent, 90 percent to 95 percent, 90 percent to 100 percent, or 95 percent to 100 percent integrate into a host tissue of the subject (e.g., a host tissue in or adjacent to a target area treated with the patch device). In some cases, 20 percent, 40 percent, 50 percent, 60 percent, 70 percent, 80 percent, 85 percent, 90 percent, 95 percent, or 100 percent integrate into a host tissue of the subject (e.g., a host tissue in or adjacent to a target area treated with the patch device). In some cases, at least 20 percent, 40 percent, 50 percent, 60 percent, 70 percent, 80 percent, 85 percent, 90 percent, or 95 percent integrate into a host tissue of the subject (e.g., a host tissue in or adjacent to a target area treated with the patch device). In some cases, at most 40 percent, 50 percent, 60 percent, 70 percent, 80 percent, 85 percent, 90 percent, 95 percent, or 100 percent integrate into a host tissue of the subject (e.g., a host tissue in or adjacent to a target area treated with the patch device).

C. Backing Components

In many cases, it is advantageous to add a backing component 130 to patch device 100 to aid in manipulation and application of the patch device. For example, the gel substrate 120 of patch device 100 is soft for direct manual manipulation, in many cases. Including a backing component 130 in the patch device (e.g., directly coupled to a first surface 160 of the gel substrate) can improve the structural integrity of the patch device during manipulation and application and can reduce the likelihood of unintentional tearing of the patch device or unintentional spatial movement of cells of the patch device relative to one another and/or relative to the outer dimensions of the gel substrate.

In many cases, the backing component 130 is fabricated separately from the gel substrate. The backing component is subsequently coupled to the first surface 160 of the gel substrate 120. A backing component 130 is coupled to the gel substrate by adding a droplet of an adhesive (e.g., biocompatible adhesive, such as a droplet of fibrin glue or hyaluronic acid) to the first surface 160 of the gel substrate 120 and applying the backing component 130 to the first surface 160 of the gel substrate 120, in some embodiments.

A backing component 130 can comprise a fibrin cap. In some cases, a fibrin cap is more dense and/or more stiff than the gel substrate 120 of a patch device 100. A fibrin cap that is more stiff than the gel substrate 120 is fabricated by increasing the concentration of fibrinogen and/or thrombin relative to the concentrations of each used to form gel substrate 120, in some embodiments. For example, a fibrin cap can comprise 20 mg/mL fibrin and 10 U/mL thrombin. In some embodiments, a backing component 130 comprises 10 mg/mL to 90 mg/mL of fibrin. In some embodiments, a backing component 130 comprises 10 mg/mL to 20 mg/mL, 10 mg/mL to 30 mg/mL, 10 mg/mL to 40 mg/mL, 10 mg/mL to 50 mg/mL, 10 mg/mL to 60 mg/mL, 10 mg/mL to 70 mg/mL, 10 mg/mL to 80 mg/mL, 10 mg/mL to 90 mg/mL, 20 mg/mL to 30 mg/mL, 20 mg/mL to 40 mg/mL, 20 mg/mL to 50 mg/mL, 20 mg/mL to 60 mg/mL, 20 mg/mL to 70 mg/mL, 20 mg/mL to 80 mg/mL, 20 mg/mL to 90 mg/mL, 30 mg/mL to 40 mg/mL, 30 mg/mL to 50 mg/mL, 30 mg/mL to 60 mg/mL, 30 mg/mL to 70 mg/mL, 30 mg/mL to 80 mg/mL, 30 mg/mL to 90 mg/mL, 40 mg/mL to 50 mg/mL, 40 mg/mL to 60 mg/mL, 40 mg/mL to 70 mg/mL, 40 mg/mL to 80 mg/mL, 40 mg/mL to 90 mg/mL, 50 mg/mL to 60 mg/mL, 50 mg/mL to 70 mg/mL, 50 mg/mL to 80 mg/mL, 50 mg/mL to 90 mg/mL, 60 mg/mL to 70 mg/mL, 60 mg/mL to 80 mg/mL, 60 mg/mL to 90 mg/mL, 70 mg/mL to 80 mg/mL, 70 mg/mL to 90 mg/mL, or 80 mg/mL to 90 mg/mL of fibrin. In some embodiments, a backing component 130 comprises 10 mg/mL, 20 mg/mL, 30 mg/mL, 40 mg/mL, 50 mg/mL, 60 mg/mL, 70 mg/mL, 80 mg/mL, or 90 mg/mL of fibrin. In some embodiments, a backing component 130 comprises at least 10 mg/mL, 20 mg/mL, 30 mg/mL, 40 mg/mL, 50 mg/mL, 60 mg/mL, 70 mg/mL, or 80 mg/mL of fibrin. In some embodiments, a backing component 130 comprises at most 20 mg/mL, 30 mg/mL, 40 mg/mL, 50 mg/mL, 60 mg/mL, 70 mg/mL, 80 mg/mL, or 90 mg/mL of fibrin. In some embodiments, the fibrin cap comprises at least 3 mg/mL of fibrin, at least 5 mg/mL of fibrin, at least 6 mg/mL of fibrin, at least 7 mg/mL of fibrin, at least 8 mg/mL of fibrin, at least 9 mg/mL of fibrin, at least 10 mg/mL of fibrin, at least 11 mg/mL of fibrin, at least 12 mg/mL of fibrin, at least 13 mg/mL of fibrin, at least 14 mg/mL of fibrin, at least 15 mg/mL of fibrin, at least 16 mg/mL of fibrin, at least 17 mg/mL of fibrin, at least 18 mg/mL of fibrin, at least 19 mg/mL of fibrin, at least 20 mg/mL of fibrin, at least 25 mg/mL of fibrin, at least 30 mg/mL of fibrin, at least 17.5 mg/mL of fibrin, at least 22.5 mg/mL of fibrin, at least 17.5 mg/mL of fibrin, at least 35 mg/mL of fibrin, at least 3 mg/mL to at most 50 mg/mL of fibrin, at least 5 mg/mL to at most 40 mg/mL of fibrin, at least 7 mg/mL to at most 35 mg/mL of fibrin, at least 9 mg/mL to at least 30 mg/mL of fibrin, at least 10 mg/mL to at least 50 mg/mL of fibrin, at least 10 mg/mL to at least 40 mg/mL of fibrin, at least 10 mg/mL to at least 30 mg/mL of fibrin, at least 10 mg/mL to at least 20 mg/mL of fibrin, at least 10 mg/mL to at least 100 mg/mL of fibrin, at least 10 mg/mL to at least 75 mg/mL of fibrin, at least 10 mg/mL to at least 60 mg/mL of fibrin, at least 10 mg/mL to at least 25 mg/mL of fibrin, at least 15 mg/mL to at least 50 mg/mL of fibrin, at least 15 mg/mL to at least 100 mg/mL of fibrin, at least 15 mg/mL to at least 45 mg/mL of fibrin, at least 15 mg/mL to at least 200 mg/mL of fibrin, at least 15 mg/mL to at least 75 mg/mL of fibrin, at least 1 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, at least 10 mg/mL to at least 50 mg/mL of fibrin, about 3 mg/mL of fibrin, about 5 mg/mL of fibrin, about 6 mg/mL of fibrin, about 7 mg/mL of fibrin, about 8 mg/mL of fibrin, about 9 mg/mL of fibrin, about 10 mg/mL of fibrin, about 11 mg/mL of fibrin, about 12 mg/mL of fibrin, about 13 mg/mL of fibrin, about 14 mg/mL of fibrin, about 15 mg/mL of fibrin, about 16 mg/mL of fibrin, about 17 mg/mL of fibrin, about 18 mg/mL of fibrin, about 19 mg/mL of fibrin, about 20 mg/mL of fibrin, about 25 mg/mL of fibrin, about 30 mg/mL of fibrin, about 17.5 mg/mL of fibrin, about 22.5 mg/mL of fibrin, and/or about 17.5 mg/mL of fibrin, about 35 mg/mL of fibrin.

In some cases, a backing component can be formed by reacting 10 U/mL thrombin and 15-80 mg/mL fibrinogen. In some embodiments, a backing component 130 is formed by combining thrombin with 10 mg/mL to 90 mg/mL of fibrinogen. In some embodiments, a backing component 130 is formed by combining thrombin with 10 mg/mL to 20 mg/mL, 10 mg/mL to 30 mg/mL, 10 mg/mL to 40 mg/mL, 10 mg/mL to 50 mg/mL, 10 mg/mL to 60 mg/mL, 10 mg/mL to 70 mg/mL, 10 mg/mL to 80 mg/mL, 10 mg/mL to 90 mg/mL, 20 mg/mL to 30 mg/mL, 20 mg/mL to 40 mg/mL, 20 mg/mL to 50 mg/mL, 20 mg/mL to 60 mg/mL, 20 mg/mL to 70 mg/mL, 20 mg/mL to 80 mg/mL, 20 mg/mL to 90 mg/mL, 30 mg/mL to 40 mg/mL, 30 mg/mL to 50 mg/mL, 30 mg/mL to 60 mg/mL, 30 mg/mL to 70 mg/mL, 30 mg/mL to 80 mg/mL, 30 mg/mL to 90 mg/mL, 40 mg/mL to 50 mg/mL, 40 mg/mL to 60 mg/mL, 40 mg/mL to 70 mg/mL, 40 mg/mL to 80 mg/mL, 40 mg/mL to 90 mg/mL, 50 mg/mL to 60 mg/mL, 50 mg/mL to 70 mg/mL, 50 mg/mL to 80 mg/mL, 50 mg/mL to 90 mg/mL, 60 mg/mL to 70 mg/mL, 60 mg/mL to 80 mg/mL, 60 mg/mL to 90 mg/mL, 70 mg/mL to 80 mg/mL, 70 mg/mL to 90 mg/mL, or 80 mg/mL to 90 mg/mL of fibrinogen. In some embodiments, a backing component 130 is formed by combining thrombin with 10 mg/mL, 20 mg/mL, 30 mg/mL, 40 mg/mL, 50 mg/mL, 60 mg/mL, 70 mg/mL, 80 mg/mL, or 90 mg/mL of fibrinogen. In some embodiments, a backing component 130 is formed by combining thrombin with at least 10 mg/mL, 20 mg/mL, 30 mg/mL, 40 mg/mL, 50 mg/mL, 60 mg/mL, 70 mg/mL, or 80 mg/mL of fibrinogen. In some embodiments, a backing component 130 is formed by combining thrombin with at most 20 mg/mL, 30 mg/mL, 40 mg/mL, 50 mg/mL, 60 mg/mL, 70 mg/mL, 80 mg/mL, or 90 mg/mL of fibrinogen. A backing component 130 comprising a fibrin cap is coupled to the gel substrate by adding a droplet of fibrin glue to the first surface 160 of the gel substrate 120 and applying the backing component to the first surface 160, in some embodiments.

In some cases, a backing component 130 comprises a bandage or dressing. In some cases, a backing component 130 comprises a mesh material. For example, a backing component 130 comprises a silicone mesh dressing (e.g., such as Adaptic Touch™ Silicone Non-Adhering Dressing), in some embodiments. In many cases, backing component 130 is flexible. A flexible backing component 130 facilitates application of patch device 100 to rounded or curved surfaces and target tissues (e.g., knees, elbows, or arms of a subject), in most embodiments. A rigid (e.g., non-flexible) backing component 130 is useful in some embodiments of the patch device 100 disclosed herein. Rigid backing components 130 allow improved structural support for the gel substrate during manipulation and application, in some cases. It is contemplated that manipulation and/or application of larger patches is facilitated by a rigid backing component 130.

In many cases, a backing component 130 (e.g., comprising a fibrin cap) can be removed after the gel substrate 120 and cellular component 110 of the patch device 100 have been applied to the target tissue 140, for example, as shown in FIG. 1B.

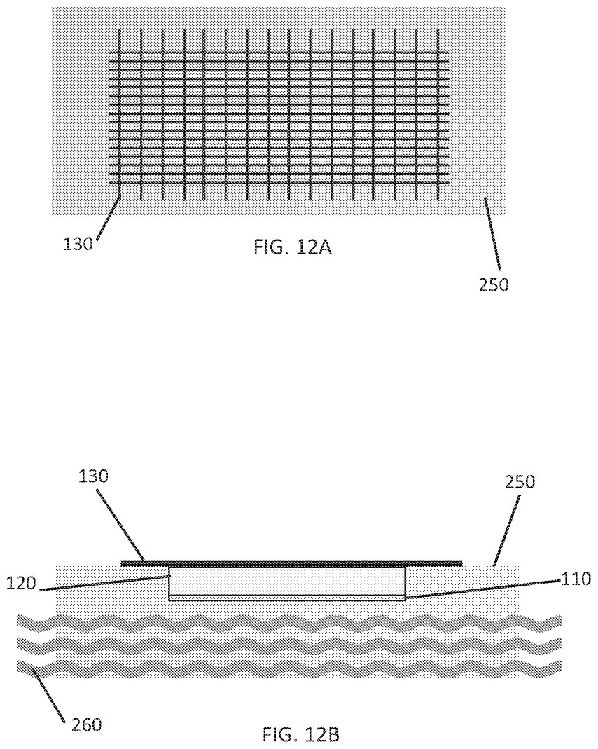

FIGS. 12A and 12B show a top view and a side view, respectively, of a patch device comprising a backing component 130 (e.g., a mesh backing component, such as a silicone mesh backing component) in a mold 250, in accordance with some embodiments. In some cases, backing component 130 is applied to a first surface of the gel substrate 120 (e.g., while the gel substrate is in mold 250). In some cases, backing component 130 is applied to a first surface of the gel substrate during the formation (e.g., gelation) of the gel substrate 120 in the mold 250. In some cases, all or a portion of the mold 250 and, optionally, the gel substrate 120 are place in a fluid 260. In some cases, fluid 260 comprises a culture medium (e.g., Melanocyte Growth Medium M2 (MGMM2)) or buffer (e.g., a cell-culture compatible buffer, such as a phosphate-buffered saline solution). In some cases, a patch device can be shipped from a first location (e.g., a manufacturing location) to a second location (e.g., a medical facility or research laboratory) in a mold 250 (e.g., as shown in FIG. 12A and/or FIG. 12B). In some cases, a kit comprises a patch device (e.g., comprising a gel substrate, and optionally, a cellular component 110 and/or a backing component 130) and a container suitable for shipping the patch device. In some cases, a container suitable for shipping the patch device is a sealable container or package (e.g., a watertight container). In some cases, a kit comprises a mold 250. In some cases, a kit comprises a patch device without a mold 250.

D. Adhesives

A patch device 100 comprises an adhesive 150 in many embodiments. An adhesive 150 causes the patch device to stick to and/or maintain its position at a target tissue. In many embodiments, an adhesive 150 is present on a second surface 170 of a patch device 100. An adhesive 150 comprises a biocompatible adhesive, in some embodiments. In many cases, an adhesive comprises a liquid or viscous reagent, such as thrombin or a fibrin glue. In some cases, the use of a biocompatible adhesive to adhere a patch device 100 to a target area of a subject (e.g., a hypopigmented or depigmented portion of the subject's skin) improves the efficiency of repigmentation of a target area. For example, an adhesive 150 of patch device 100 reduces the movement of a patch device or portion thereof relative to an area of the subject's body to which the patch device is applied (e.g., the area of skin being treated with the patch device). Reducing the movement of a patch device or portion thereof relative to the area of the subject's body to which the patch device is applied improves the efficiency of transfer of cells of cellular component 110 to the target area, in many cases. In many cases, reducing the movement of the patch device or a portion thereof relative to the area of the subject's body to which the patch device is applied improves the integration of cells of the cellular component 110 into the target area.

In some embodiments, an adhesive 150 of patch device 100 comprises fibrin in a concentration of 1 mg/mL to 10 mg/mL. In some embodiments, an adhesive 150 of patch device 100 comprises fibrin in a concentration of 1 mg/mL to 2 mg/mL, 1 mg/mL to 3 mg/mL, 1 mg/mL to 4 mg/mL, 1 mg/mL to 5 mg/mL, 1 mg/mL to 6 mg/mL, 1 mg/mL to 7 mg/mL, 1 mg/mL to 8 mg/mL, 1 mg/mL to 9 mg/mL, 1 mg/mL to 10 mg/mL, 2 mg/mL to 3 mg/mL, 2 mg/mL to 4 mg/mL, 2 mg/mL to 5 mg/mL, 2 mg/mL to 6 mg/mL, 2 mg/mL to 7 mg/mL, 2 mg/mL to 8 mg/mL, 2 mg/mL to 9 mg/mL, 2 mg/mL to 10 mg/mL, 3 mg/mL to 4 mg/mL, 3 mg/mL to 5 mg/mL, 3 mg/mL to 6 mg/mL, 3 mg/mL to 7 mg/mL, 3 mg/mL to 8 mg/mL, 3 mg/mL to 9 mg/mL, 3 mg/mL to 10 mg/mL, 4 mg/mL to 5 mg/mL, 4 mg/mL to 6 mg/mL, 4 mg/mL to 7 mg/mL, 4 mg/mL to 8 mg/mL, 4 mg/mL to 9 mg/mL, 4 mg/mL to 10 mg/mL, 5 mg/mL to 6 mg/mL, 5 mg/mL to 7 mg/mL, 5 mg/mL to 8 mg/mL, 5 mg/mL to 9 mg/mL, 5 mg/mL to 10 mg/mL, 6 mg/mL to 7 mg/mL, 6 mg/mL to 8 mg/mL, 6 mg/mL to 9 mg/mL, 6 mg/mL to 10 mg/mL, 7 mg/mL to 8 mg/mL, 7 mg/mL to 9 mg/mL, 7 mg/mL to 10 mg/mL, 8 mg/mL to 9 mg/mL, 8 mg/mL to 10 mg/mL, or 9 mg/mL to 10 mg/mL. In some embodiments, an adhesive 150 of patch device 100 comprises fibrin in a concentration of 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments, an adhesive 150 of patch device 100 comprises fibrin in a concentration of at least 1 mg/mL, 2 mg/mL, 3 mg/mL, 4 mg/mL, 5 mg/mL, 6 mg/mL, 7 mg/mL, 8 mg/mL, or 9 mg/mL. In some embodiments, an adhesive 150 of patch device 100 comprises fibrin in a concentration of at most 2 mg/mL, 3 mg/mL, 4 mg/mL, 5 mg/mL, 6 mg/mL, 7 mg/mL, 8 mg/mL, 9 mg/mL, or 10 mg/mL.

In some embodiments, an adhesive 150 of patch device 100 comprises thrombin in a concentration of 0.1 U/mL to 10 U/mL. In some embodiments, an adhesive 150 of patch device 100 comprises thrombin in a concentration of 0.1 U/mL to 1 U/mL, 0.1 U/mL to 1.5 U/mL, 0.1 U/mL to 2 U/mL, 0.1 U/mL to 2.5 U/mL, 0.1 U/mL to 3 U/mL, 0.1 U/mL to 4 U/mL, 0.1 U/mL to 5 U/mL, 0.1 U/mL to 7.5 U/mL, 0.1 U/mL to 10 U/mL, 1 U/mL to 1.5 U/mL, 1 U/mL to 2 U/mL, 1 U/mL to 2.5 U/mL, 1 U/mL to 3 U/mL, 1 U/mL to 4 U/mL, 1 U/mL to 5 U/mL, 1 U/mL to 7.5 U/mL, 1 U/mL to 10 U/mL, 1.5 U/mL to 2 U/mL, 1.5 U/mL to 2.5 U/mL, 1.5 U/mL to 3 U/mL, 1.5 U/mL to 4 U/mL, 1.5 U/mL to 5 U/mL, 1.5 U/mL to 7.5 U/mL, 1.5 U/mL to 10 U/mL, 2 U/mL to 2.5 U/mL, 2 U/mL to 3 U/mL, 2 U/mL to 4 U/mL, 2 U/mL to 5 U/mL, 2 U/mL to 7.5 U/mL, 2 U/mL to 10 U/mL, 2.5 U/mL to 3 U/mL, 2.5 U/mL to 4 U/mL, 2.5 U/mL to 5 U/mL, 2.5 U/mL to 7.5 U/mL, 2.5 U/mL to 10 U/mL, 3 U/mL to 4 U/mL, 3 U/mL to 5 U/mL, 3 U/mL to 7.5 U/mL, 3 U/mL to 10 U/mL, 4 U/mL to 5 U/mL, 4 U/mL to 7.5 U/mL, 4 U/mL to 10 U/mL, 5 U/mL to 7.5 U/mL, 5 U/mL to 10 U/mL, or 7.5 U/mL to 10 U/mL. In some embodiments, an adhesive 150 of patch device 100 comprises thrombin in a concentration of 0.1 U/mL, 1 U/mL, 1.5 U/mL, 2 U/mL, 2.5 U/mL, 3 U/mL, 4 U/mL, 5 U/mL, 7.5 U/mL, or 10 U/mL. In some embodiments, an adhesive 150 of patch device 100 comprises thrombin in a concentration of at least 0.1 U/mL, 1 U/mL, 1.5 U/mL, 2 U/mL, 2.5 U/mL, 3 U/mL, 4 U/mL, 5 U/mL, or 7.5 U/mL. In some embodiments, an adhesive 150 of patch device 100 comprises thrombin in a concentration of at most 1 U/mL, 1.5 U/mL, 2 U/mL, 2.5 U/mL, 3 U/mL, 4 U/mL, 5 U/mL, 7.5 U/mL, or 10 U/mL.

In some cases, an adhesive 150 comprises a thickening agent, such as hyaluronic acid. In some embodiments, an adhesive 150 of patch device 100 comprises hyaluronic acid in a concentration of 0.1 mg/mL to 2 mg/mL. In some embodiments, an adhesive 150 of patch device 100 comprises hyaluronic acid in a concentration of 0.1 mg/mL to 0.5 mg/mL, 0.1 mg/mL to 1 mg/mL, 0.1 mg/mL to 1.5 mg/mL, 0.1 mg/mL to 2 mg/mL, 0.5 mg/mL to 1 mg/mL, 0.5 mg/mL to 1.5 mg/mL, 0.5 mg/mL to 2 mg/mL, 1 mg/mL to 1.5 mg/mL, 1 mg/mL to 2 mg/mL, or 1.5 mg/mL to 2 mg/mL. In some embodiments, an adhesive 150 of patch device 100 comprises hyaluronic acid in a concentration of 0.1 mg/mL, 0.5 mg/mL, 1 mg/mL, 1.5 mg/mL, or 2 mg/mL. In some embodiments, an adhesive 150 of patch device 100 comprises hyaluronic acid in a concentration of at least 0.1 mg/mL, 0.5 mg/mL, 1 mg/mL, or 1.5 mg/mL. In some embodiments, an adhesive 150 of patch device 100 comprises hyaluronic acid in a concentration of at most 0.5 mg/mL, 1 mg/mL, 1.5 mg/mL, or 2 mg/mL.

An adhesive 150 comprises more than one molecule, in some cases. For example, in some cases, an adhesive 150 comprises fibrin and thrombin. In some embodiments, an adhesive 150 of patch device 100 comprises 2 U/mL of thrombin and fibrin in a concentration of 1 mg/mL to 10 mg/mL. In some embodiments, an adhesive 150 of patch device 100 comprises 2 U/mL of thrombin and fibrin in a concentration of 1 mg/mL to 2 mg/mL, 1 mg/mL to 3 mg/mL, 1 mg/mL to 4 mg/mL, 1 mg/mL to 5 mg/mL, 1 mg/mL to 6 mg/mL, 1 mg/mL to 7 mg/mL, 1 mg/mL to 8 mg/mL, 1 mg/mL to 9 mg/mL, 1 mg/mL to 10 mg/mL, 2 mg/mL to 3 mg/mL, 2 mg/mL to 4 mg/mL, 2 mg/mL to 5 mg/mL, 2 mg/mL to 6 mg/mL, 2 mg/mL to 7 mg/mL, 2 mg/mL to 8 mg/mL, 2 mg/mL to 9 mg/mL, 2 mg/mL to 10 mg/mL, 3 mg/mL to 4 mg/mL, 3 mg/mL to 5 mg/mL, 3 mg/mL to 6 mg/mL, 3 mg/mL to 7 mg/mL, 3 mg/mL to 8 mg/mL, 3 mg/mL to 9 mg/mL, 3 mg/mL to 10 mg/mL, 4 mg/mL to 5 mg/mL, 4 mg/mL to 6 mg/mL, 4 mg/mL to 7 mg/mL, 4 mg/mL to 8 mg/mL, 4 mg/mL to 9 mg/mL, 4 mg/mL to 10 mg/mL, 5 mg/mL to 6 mg/mL, 5 mg/mL to 7 mg/mL, 5 mg/mL to 8 mg/mL, 5 mg/mL to 9 mg/mL, 5 mg/mL to 10 mg/mL, 6 mg/mL to 7 mg/mL, 6 mg/mL to 8 mg/mL, 6 mg/mL to 9 mg/mL, 6 mg/mL to 10 mg/mL, 7 mg/mL to 8 mg/mL, 7 mg/mL to 9 mg/mL, 7 mg/mL to 10 mg/mL, 8 mg/mL to 9 mg/mL, 8 mg/mL to 10 mg/mL, or 9 mg/mL to 10 mg/mL. In some embodiments, an adhesive 150 of patch device 100 comprises 2 U/mL of thrombin and fibrin in a concentration of 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments, an adhesive 150 of patch device 100 comprises 2 U/mL of thrombin and fibrin in a concentration of at least 1 mg/mL, 2 mg/mL, 3 mg/mL, 4 mg/mL, 5 mg/mL, 6 mg/mL, 7 mg/mL, 8 mg/mL, or 9 mg/mL. In some embodiments, an adhesive 150 of patch device 100 comprises 2 U/mL of thrombin and fibrin in a concentration of at most 2 mg/mL, 3 mg/mL, 4 mg/mL, 5 mg/mL, 6 mg/mL, 7 mg/mL, 8 mg/mL, 9 mg/mL, or 10 mg/mL. In some cases, an adhesive 150 comprises fibrin and hyaluronic acid. In some cases, an adhesive 150 comprises thrombin and hyaluronic acid. In some cases, an adhesive 150 comprises fibrin, thrombin, and hyaluronic acid. For example, an adhesive 150 comprises fibrin and 1 mg/mL of hyaluronic acid, in some embodiments. In some embodiments, an adhesive 150 comprises thrombin and 1 mg/mL of hyaluronic acid. In some embodiments, an adhesive 150 comprises fibrin, thrombin, and 1 mg/mL of hyaluronic acid.

In the present detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### II. Methods of Manufacturing

Disclosed herein are methods of manufacturing (e.g., fabricating) a patch device 100. In some cases, providing a patch device 100 comprises manufacturing (e.g., fabricating) the patch device. In many embodiments, a method of treating a subject (e.g., a subject having hypopigmentation or depigmentation of the skin) comprises providing a patch device 100 (e.g., a patch device as disclosed above). As disclosed above, the method of manufacturing a patch device 100 can affect one or more properties of the patch device (e.g., spatial distribution of cells within the patch device, deformability of the patch device, and/or mechanical stiffness of the patch device). In many cases, manufacturing a patch device according to the present disclosure can improve efficiency of a treatment of a patient having hypopigmentation or depigmentation, for example, by improving the efficiency with which cells are transferred to a target area (e.g., by controlling the spatial distribution of cells within the patch device, by increasing the contact area between the patch device and the target area, and/or by maintaining the spatial position of the cells of the patch device and the target area during transfer of the cells from the patch device to the target area).

In some embodiments, two or more reagents disclosed herein (e.g., fibrinogen, thrombin, and/or all or a portion of cellular component 110) are mixed to form the gel substrate 120 of patch device 100. In some cases, a method of fabricating (e.g., forming) a gel substrate 120 comprises mixing two or more reagents (e.g., fibrinogen, thrombin, and or all or a portion of cellular component 110) in a container (e.g., a mold, such as a negative mold 250). In some cases, the gel substrate 120 is formed into a desired shape by the shape of the container in which it is mixed and allowed to gel. In some cases, a mold 250 for fabricating a patch device 100 or a portion thereof (e.g., gel substrate 120) has a regular cross-sectional shape (e.g., a rectangle, a square, an oval, a circle, a triangle, a pentagon, or a trapezoid). In some cases, a mold 250 for fabricating a patch device 100 or a portion thereof (e.g., gel substrate 120) has an irregular cross-sectional shape (e.g., a shape corresponding to a target area of a subject to be treated).

In some cases, a mold 250 (e.g., a negative mold) is fabricated using a positive mold 270. For example, a solution or suspension used to form a mold 250 (e.g., an agarose solution, such as a 2% agarose solution) can be added to (e.g., poured into) a positive mold 270 and allowed to set (e.g., as shown in FIG. 13A). In some cases, a mold (e.g., a negative mold 250) is allowed to set in a positive mold 270 at room temperature (e.g., 23° C.), at 4° C., at 0° C., at −20° C., or at −80° C., at a temperature greater than 23° C., at a temperature from 23° C. to 4° C., at a temperature from 4° C. to 0° C., at a temperature from 0° C. to −20° C., or at a temperature from −20° C. to −80° C. In some cases, a mold (e.g., a negative mold 250) is allowed to set in a positive mold 270 for 1 minute to 5 minutes, for 5 minutes to 10 minutes, 10 minutes to 15 minutes, 15 minutes to 30 minutes, 30 minutes to 1 hour, for 1 hour to 2 hours, for 2 hours to 4 hours, for 4 hours to 8 hours, for 8 hours to 12 hours, overnight, for 12 hours to 24 hours, for 1 minute to 24 hours, for 1 hour to 24 hours, for greater than 24 hours, or for less than 1 minute. For example, a mold 250 is allowed to set (e.g., gel) at room temperature (e.g., at a temperature from 20° C. to 25° C.) for 1 hour, in some embodiments. In some cases, a positive mold 270 is sterilizable (e.g., by autoclaving or exposure to soap, bleach, and/or alcohol solutions). In some cases, a positive mold 270 comprises a metal material. In some cases, a positive mold 270 comprises a plastic material. In some cases, a positive mold 270 comprises a shape, three-dimensional pattern, and/or surface contour that is desired for a gel substrate 120. For example, a portion of the inner surface of the positive mold 270 can comprise an indentation comprising a shape, three-dimensional pattern, and or surface contour that is desired for the final gel substrate 120 (e.g., based on the application for which the patch device is to be used, such as the location, shape, or size of the target area, as described herein), for example, as shown in FIG. 13B.

As shown in FIG. 13B, a mold 250 is removed from positive mold 270 (e.g., after gelation of mold 250), in some embodiments. In some cases, mold 250 is placed in a container (e.g., a petri dish, as shown in FIG. 13B) for subsequent patch device fabrication steps, such as formation of the gel substrate in mold 250. Positive mold 270 can be used to create a plurality of identical molds 250, for example, to allow for simultaneous comparison of patch devices comprising identical gel substrates, for fabrication of identical gel substrates to be used at different target areas (e.g., in the same or different patients), or sequentially at the same target area on a single subject. In some cases, a fluid 260 is added to the container (e.g., petri dish), for example, to keep the gel substrate and/or cellular component hydrated and/or to provide the cells of the cellular component with nutrients.

In some embodiments, fabricating a patch device 100 comprises forming a gel substrate 120 into a regular or irregular three-dimensional shape (e.g., by using a mold 250 (e.g., a negative mold) shaped to define the regular or irregular three-dimensional shape). In some cases, gel substrate 120 comprises a constant cross-sectional shape from first surface 160 to second surface 170. For example, gel substrate can comprise a substantially circular, a substantially rectangular, a substantially square, a substantially triangular, a substantially oval, or a substantially trapezoidal cross-sectional shape (e.g., in transverse x-y plane 240), in some cases. For example, a gel substrate 120 comprises a rectangular cross-sectional shape having a width in an x-axis direction of 5-10 millimeters, a length of 10 millimeters in a y-axis direction perpendicular to the x-axis direction, and a height of 2 millimeters in a z-axis direction. In some embodiments, gel substrate 120 is formed into a desired shape by mixing two or more reagents (e.g., fibrinogen, thrombin, and/or cellular component 110) in a mold 250. In some cases, forming gel substrate into a specific shape (e.g., a shape that matches all or a portion of a target area of a subject's skin) improves the efficiency of repigmentation (e.g., by decreasing waste from trimming patch device 100 to match the target area) and/or improves the quality of repigmentation (e.g., by matching the shape pigmentation of a portion of the subject's skin surrounding the target area).

In some cases, fabricating a patch device 100 comprises cutting patch device 100 (or portion thereof, such as gel substrate 120) into a desired shape after formation of the gel substrate 120. For example, a square- or rectangular-shaped patch device 100 or gel substrate 120 is cut into two or more smaller patch devices 100 or gel substrates 120, in some embodiments. In some cases, the two or more smaller patch devices 100 or gel substrates 120 are the same shape as the larger patch device or gel substrate from which they were cut. In some cases, the two or more smaller patch devices 100 or gel substrates 120 comprise one or more different shapes than the larger patch device or gel substrate from which they were cut.

In some cases, fabricating a patch device comprises mixing fibrinogen with thrombin (e.g., in quantities and ratios described herein). In many cases, fibrinogen is converted to fibrin during formation of gel substrate 120 through an enzymatic reaction with thrombin, which results from mixing fibrinogen and thrombin in the amounts and ratios disclosed herein at temperature ranges disclosed herein. The rate at which fibrinogen is enzymatically cleaved (e.g., digested) by thrombin into fibrin, the extent to which the fibrinogen is cleaved into fibrin, and the rate at which the gel substrate congeals depends on the amount of fibrinogen, the activity of thrombin that is used, the pH of the gel substrate as it congeals, the concentration of cells present, and the temperature of the mixture. For example, thrombin exhibits its highest activity at pH 8.3 to 9.5. In some cases, the formation of a gel substrate is performed at a temperature of about 4 degrees Celsius to about 30 degrees Celsius. In general, the formation of the gel substrate will proceed more slowly when the gel is formed at a temperature closer to 4 degrees Celsius than 30 degrees Celsius. In some cases, a method of fabricating patch device 100 comprises mixing fibrinogen and thrombin (e.g., to allow the thrombin to cleave the fibrinogen) at room temperature (e.g., approximately 23-25 degrees Celsius) for 1 to 15 minutes, 15 to 45 minutes, 20 to 30 minutes, or 45 to 60 minutes. In some embodiments, a method of fabricating patch device 100 or a portion thereof (e.g., an adhesive comprising a fibrin glue) comprises mixing fibrinogen and thrombin at room temperature for less than 5 seconds, less than 10 seconds, less than 20 seconds, less than 25 seconds, less than 30 seconds, less than 45 seconds, or less than 1 minute, for example, when thrombin is used at 5 to 10 U/ml. In some cases, distribution of cells within the gel substrate 120 is affected by the rate at which the gel substrate forms. For example, gel substrates formed in about a minute or less can result in cells applied to the top of the gel substrate mixture being distributed higher within the gel substrate (e.g., in a z-axis direction relative to the first surface and the second surface). Advantageous cell distribution within the gel substrate can be realized when 2 U/ml of thrombin is mixed with 10 mg/mL to 15 mg/mL of fibrinogen at room temperature for 20 to 30 minutes (e.g., before being moved to 4 degrees Celsius for storage). In many cases, a method of fabricating a patch device comprises missing 2 U/mi of thrombin with 10 mg/mL to 15 mg/mL of fibrinogen at room temperature for 20 to 30 minutes. In many cases, a method of fabricating a patch device comprises storing a patch device 100 or portion thereof (e.g., a gel substrate 120 such as a gel substrate 120 comprising cellular component 110) at a temperature of 4 degrees Celsius for less than one hour, for one hour to two hours, for two hours to four hours, for four hours to 8 hours, for 8 hours to 12 hours, for 12 hours to 16 hours, for 16 hours to 20 hours, for 20 hours to 24 hours, for 24 to 72 hours, or for more than 72 hours. In some cases, a method of fabricating a patch device comprises storing a patch device 100 or portion thereof (e.g., a gel substrate 120 such as a gel substrate 120 comprising cellular component 110) overnight (e.g., approximately 8 to 16 hours).

In some embodiments, a method of fabricating gel substrate 120 comprises adding cells (e.g., all or a portion of cellular component 110) to one or more reagents (e.g., thrombin and/or fibrinogen) used to form gel substrate 120. In some cases, a method of fabricating gel substrate 120 comprises adding cells (e.g., all or a portion of cellular component 110) to one or more reagents (e.g., thrombin and/or fibrinogen) before gel substrate 120 is formed (e.g., congealed). For example, a suspension of isolated cells (e.g., comprising pigment-producing cells, such as melanocytes) in culture medium (e.g., MGMM2 or M254) are mixed with fibrinogen (e.g., a solution of 40 mg/mL fibrinogen) and thrombin (e.g., a 4 U/mL preparation of thrombin) in a 1:1:2 volumetric ratio in a mold 250, in some embodiments of a method for forming a gel substrate 120 comprising a cellular component 110.

In some cases, a method of fabricating gel substrate 120 comprises adding cells (e.g., all or a portion of the cells comprising cellular component 110) to a surface (e.g., first surface 160) of gel substrate 120 during fabrication of patch device 100. In some cases, the cells are applied to the surface (e.g., first surface 160) of gel substrate 120 in a liquid suspension (e.g., an aqueous suspension, such as a suspension of cells in culture medium). In some cases, the fibrinogen and thrombin are mixed before a step comprising adding the cells to the surface of the gel substrate. In some cases, the gel substrate is not fully congealed when the cell suspension is added to the top of the fibrinogen and thrombin mixture. In some cases, the only cells in the cell suspension are pigment-producing cells, such as melanocytes. In some cases, the cell suspension comprises pigment-producing cells and another cell type. For example, the cell suspension can comprise melanocytes and keratinocytes.

In some cases, a method of fabricating (e.g., manufacturing) patch device 100 comprises culturing at least a portion of the cells comprising cellular component 110 (e.g., prior to adding the portion of the plurality of melanocytes to a gel substrate 120 of patch device 100). In some cases, a method of fabricating (e.g., manufacturing) patch device 100 comprises culturing at least a portion of the cells comprising cellular component 110 on tissue culture plastic. In some cases, a method of fabricating (e.g., manufacturing) patch device 100 comprises culturing at least a portion of a plurality of melanocytes (e.g., prior to adding the portion of the plurality of melanocytes to a gel substrate 120 of patch device 100). In some cases, a method of fabricating (e.g., manufacturing) patch device 100 comprises culturing at least a portion of the cells comprising cellular component 110 for at least 1, at least 2, at least 3, at least 4, at least 5, at least 6, at least 7, at least 8, at least 9, at least 10, at least 15, or at least 20 passages. In some cases, a method of fabricating (e.g., manufacturing) patch device 100 comprises culturing at least a portion of the cells comprising cellular component 110 (e.g., at least a portion of the plurality of melanocytes comprising cellular component 110) for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 population doublings.

In many cases, a method of fabricating a patch device 100 comprises applying a backing component 130 to the patch device (e.g., first surface 160 of the patch device). In some cases, a backing component 130 (e.g., a silicone dressing or fibrin cap) is applied to the first surface 160 (e.g., top surface) of the gel substrate during or after gel substrate formation using forceps. In some cases, a method of fabricating a patch device comprises applying a backing component 130 to a patch device (e.g., first surface 160 of gel substrate 120) after all or a portion of cellular component 110 has been added to gel substrate 120. In some cases, a method of fabricating a patch device comprises applying a backing component 130 to a patch device (e.g., first surface 160 of gel substrate 120) after the gel substrate has congealed. In some cases, a small amount of fibrin glue or thrombin is applied to the first surface 160 of gel substrate 120 in order to improve the coupling of backing component 130 to the first surface 160 of gel substrate 120. In some cases, a backing component 130 is applied to the patch device (e.g., to the first surface of the patch device) while the gel substrate and, optionally, the cellular component is in mold 250, as shown in FIG. 13C and FIG. 13D.

In many cases, a method of fabricating a patch device 100 comprises applying an adhesive 150 to patch device 100. For example, a method of fabricating a patch device 100 comprises applying adhesive 150 to second surface 170 of patch device 100, in many cases. In some cases, a method of fabricating a patch device 100 comprises placing the patch device 100 in a package (e.g., a package for transport or commercial sale). In some cases, a patch device comprising a gel substrate, a population of cells, and, optionally, a backing component 130 and/or a mold 250 (e.g., as shown in FIG. 13D), can be shipped from a first location to a second location (e.g., from a fabrication room, such as in a factory or a first laboratory, to a destination for use, such as a clinical facility or second laboratory in which experiments are performed). In some cases, the package is the mold 250 in which the gel substrate was formed. In some cases, the package is not the mold 250 in which the gel substrate was formed. In some cases, fabricating a patch device 100 comprises applying an adhesive 150 to patch device 100 before patch device 100 is placed in a package (e.g., a package for transport or commercial sale). In some cases, applying adhesive 150 to patch device 100 before patch device 100 is placed in a package (e.g., a package for transport or commercial sale) reduces the number of steps required when the patch device is used (e.g., by avoiding the need to apply adhesive during a procedure involving administering the patch device to a target area of a subject). In some cases, fabricating a patch device 100 comprises applying an adhesive 150 to patch device 100 (e.g., second surface 170 of patch device 100) after removing patch device 100 from a package (e.g., a package for transport or commercial sale). In some cases, applying adhesive 150 to patch device 100 after removing patch device 100 from a package reduces the amount of drying and/or solidification experienced by the adhesive 150 before use.

### III. Methods of Treating a Subject Having Skin Hypopigmentation or Depigmentation

Disclosed herein are methods and devices for treating skin depigmentation and skin hypopigmentation in a subject. For example, methods and devices disclosed herein are useful for the treatment of depigmentation and hypopigmentation resulting from disease (e.g., vitiligo, piebaldism, tinea versicolor etc.) or injury (e.g., scars, burns, or skin ulcers, such as diabetic wounds). Vitiligo is a skin condition in which portions of a subject's skin appear noticeably lighter in pigmentation (e.g., whiter) from other portions. Vitiligo results from a lack of melanocytes in a portion of the epidermis of a subject's skin (e.g., as compared to a non-affected portion of the subject's skin. Melanocytes produce melanin, which is primarily responsible for skin pigmentation.

In many cases, existing systems and methods for repigmentation of skin result in uneven or undesired distribution of pigmentation at a target area of a subject after treatment. In some cases, this results from a lack of control over the distribution of pigment-producing during and after transfer to the target area of the subject and can necessitate additional interventions (e.g., additional applications of pigment-producing cells) to obtain a desired distribution of pigmentation at the target area (e.g., a more even distribution of pigmentation at the target area).

The control over distribution of pigment-producing cells of the cellular component 110 within gel substrate 120 resulting from the methods of manufacture and structure and components of patch devices disclosed herein allow practitioners to maintain the desired spatial distribution of the pigment-producing cells within gel substrate 120, in many embodiments. In many cases, this results in improved distribution of pigmentation at a target area, which can in turn result in a reduction to the number of times patch devices disclosed herein need to be applied, for example, relative to existing skin repigmentation technologies.

In many cases, a method of treating a subject in need thereof (e.g., a subject having hypopigmentation or depigmentation of the skin) comprises providing a patch device 100 disclosed herein. In many cases, a method of treating a subject in need thereof (e.g., a subject having hypopigmentation or depigmentation of the skin) comprises fabricating a patch device 100 disclosed herein. In many cases, a method of treating a subject (e.g., a target area of a subject, such as a portion of the subject's skin having hypopigmentation or depigmentation) comprises delivering a population (e.g., all or a portion of cellular component 110) of cells (e.g., melanocytes) to the target area. In many cases, a method of treating a subject (e.g., a target area of a subject, such as a portion of the subject's skin having hypopigmentation or depigmentation) comprises delivering a population of pigment-producing cells (e.g., melanocytes) to the target area. In many cases, a method of treating a subject (e.g., a target area of a subject, such as a portion of skin having hypopigmentation or depigmentation) comprises administering a patch device 100 to the subject. In many cases, administering a patch device 100 comprises contacting a subject (e.g., a target area of the subject, such as a portion of the subject's skin having hypopigmentation or depigmentation) with at least a portion of patch device 100 (e.g., adhesive 150 and/or second surface 170 of gel substrate 120). In many cases, a method of treating a subject comprises delivering pigmented cells to a target area (e.g., skin surface) of a subject having hypopigmentation or depigmentation. In many cases, a method of treating a subject comprises substantially even distribution of pigment-producing cells to a target area of a subject (e.g., a non-pigmented portion of the subject's skin).

In some cases, a method of treating a subject in need thereof comprises culturing at least a portion of the cells comprising cellular component 110 of patch device 100 (e.g., prior to adding the portion of the plurality of melanocytes to a gel substrate 120 of patch device 100). In some cases, a method of treating a subject in need thereof comprises culturing at least a portion of the cells comprising cellular component 110 on tissue culture plastic. In some cases, a method of treating a subject in need thereof comprises culturing at least a portion of a plurality of melanocytes (e.g., prior to adding the portion of the plurality of melanocytes to a gel substrate 120 of patch device 100). In some cases, a method of treating a subject in need thereof comprises culturing at least a portion of the cells comprising cellular component 110 for at least 1, at least 2, at least 3, at least 4, at least 5, at least 6, at least 7, at least 8, at least 9, at least 10, at least 15, or at least 20 passages. In some cases, a method of treating a subject in need thereof comprises culturing at least a portion of the cells comprising cellular component 110 (e.g., at least a portion of the plurality of melanocytes comprising cellular component 110) for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 population doublings.

In many cases, a method of treating a subject in need thereof comprises maintaining the spatial distribution of cells (e.g., pigment-producing cells) comprising the cellular component 110 within gel substrate 120 of patch device 100. In many cases, maintaining the spatial distribution of cells (e.g., pigment-producing cells) of cellular component 110 within gel substrate 120 comprises fabricating (e.g., manufacturing) patch device 100 or a portion thereof (e.g., gel substrate 120) according to the specifications of the present application. For example, spatial distribution of cells (e.g., pigment-producing cells) of cellular component 110 is maintained by adding the cells (e.g., pigment-producing cells) to a gel substrate 120 comprising a combination of molecules (e.g., thrombin and fibrinogen) in amounts of each disclosed herein, in many embodiments.

In many cases, a method of treating a subject in need thereof comprises preparing a target area (e.g., a portion of a subject's skin having hypopigmentation or depigmentation) before application the of patch device 100. In some cases, preparing a target area comprises performing one or more of the following steps: (a) shaving or depilating the target area, (b) washing the target area, and/or (c) sterilizing the target area (e.g., using an antimicrobial agent, such as an agent comprising soap, chlorhexidine gluconate, iodine, an iodophor, ethanol, isopropyl alcohol, or triclosan). In some cases, preparing a target area (e.g., a skin surface of a subject) comprises de-epithelializing the target area (e.g., via dissection or mechanical abrasion) and/or treating the site with liquid nitrogen (e.g., to induce blister formation), negative pressure (e.g., to induce suction blister formation), psoralen and/or ultraviolet A light (e.g., psoralen plus ultraviolet A light), or laser light.

In some cases, a method comprises removing patch device 100 from a storage location (e.g., a package or mold 250, for example, as shown in FIG. 13E and FIG. 13F) after fabrication of a patch device 100. In some cases, a method for treating a subject comprises applying patch device 100 to at least a portion of a target area of a subject (e.g., a hypopigmented or depigmented portion of the subject's skin). In many embodiments, applying patch device 100 to at least a portion of a target area of a subject comprises contacting at least a portion of the target area with the second surface 170 and/or an adhesive 150 (e.g., fibrin glue or hyaluronic acid) applied to second surface 170 (e.g., placing second surface 170 and/or or adhesive 150 in direct contact with at least a portion of the target area). Cell transfer can be improved in cases where cells are disposed within the gel substrate 120 of patch device 100 in close proximity to a second surface 170 (e.g., as disclosed herein) that is applied to the target area. In some cases, treating a subject in need thereof comprises manufacturing a gel substrate 120 of patch device 100 such that at least 50%, at least 75%, at least 80%, at least 90%, at least 95%, at least 99%, or at least 100% of the cells of cellular component 110 are disposed within a distance 190 of second surface 170 of patch device 100 that is at most 50 percent, at most 45 percent, 40 percent, 35 percent, 30 percent, 25 percent, 20 percent, 15 percent, 10 percent, or 5 percent of a height 180 of the gel substrate, as disclosed herein. In some cases, patch device 100 is manipulated by grasping a backing component 130 of the patch device (e.g., using forceps). In some cases, a method of treating a subject comprises removing backing component 130 from the gel substrate 120 (e.g., by peeling the backing component off of first surface 160 of gel substrate 120). In some cases, removing backing component 130 from gel substrate 120 advantageously decreases the profile of the applied patch device and/or decreases the likelihood that the patch device will shift position relative to the target area if the backing component is subjected to an incidental shear force.

In some cases, a method of treating a subject in need thereof comprises applying a perpendicular force or pressure (e.g., a compressive force or pressure) from patch device 100 to the surface of the target area (e.g., during and/or after application of patch device 100 to the target area of a subject). Applying a perpendicular force or pressure (e.g., a compressive force or pressure or normal to a subject's skin surface) from patch device 100 to a target area (e.g., with even pressure across the patch device and in a direction perpendicular to a flat or curved target area at points all along the patch device) improves the efficiency and homogeneity of cell transfer from patch device 100 to the target area, in many cases. In some cases, applying a pressure or force to a patch device 100 that is perpendicular to the subject's skin surface comprises applying a pressure or force (e.g., a compressive pressure or force) to 20 percent to 100 percent of backing component 130 or first surface 160 of patch device 100. In some cases, applying a pressure or force to a patch device 100 that is perpendicular to the subject's skin surface comprises applying a pressure or force (e.g., a compressive pressure or force) to 20 percent to 40 percent, 20 percent to 50 percent, 20 percent to 60 percent, 20 percent to 70 percent, 20 percent to 80 percent, 20 percent to 85 percent, 20 percent to 90 percent, 20 percent to 95 percent, 20 percent to 100 percent, 40 percent to 50 percent, 40 percent to 60 percent, 40 percent to 70 percent, 40 percent to 80 percent, 40 percent to 85 percent, 40 percent to 90 percent, 40 percent to 95 percent, 40 percent to 100 percent, 50 percent to 60 percent, 50 percent to 70 percent, 50 percent to 80 percent, 50 percent to 85 percent, 50 percent to 90 percent, 50 percent to 95 percent, 50 percent to 100 percent, 60 percent to 70 percent, 60 percent to 80 percent, 60 percent to 85 percent, 60 percent to 90 percent, 60 percent to 95 percent, 60 percent to 100 percent, 70 percent to 80 percent, 70 percent to 85 percent, 70 percent to 90 percent, 70 percent to 95 percent, 70 percent to 100 percent, 80 percent to 85 percent, 80 percent to 90 percent, 80 percent to 95 percent, 80 percent to 100 percent, 85 percent to 90 percent, 85 percent to 95 percent, 85 percent to 100 percent, 90 percent to 95 percent, 90 percent to 100 percent, or 95 percent to 100 percent of backing component 130 or first surface 160 of patch device 100. In some cases, applying a pressure or force to a patch device 100 that is perpendicular to the subject's skin surface comprises applying a pressure or force (e.g., a compressive pressure or force) to 20 percent, 40 percent, 50 percent, 60 percent, 70 percent, 80 percent, 85 percent, 90 percent, 95 percent, or 100 percent of backing component 130 or first surface 160 of patch device 100. In some cases, applying a pressure or force to a patch device 100 that is perpendicular to the subject's skin surface comprises applying a pressure or force (e.g., a compressive pressure or force) to at least 20 percent, 40 percent, 50 percent, 60 percent, 70 percent, 80 percent, 85 percent, 90 percent, or 95 percent of backing component 130 or first surface 160 of patch device 100. In some cases, applying a pressure or force to a patch device 100 that is perpendicular to the subject's skin surface comprises applying a pressure or force (e.g., a compressive pressure or force) to at most 40 percent, 50 percent, 60 percent, 70 percent, 80 percent, 85 percent, 90 percent, 95 percent, or 100 percent of backing component 130 or first surface 160 of patch device 100. In some cases, applying a pressure or force from patch device 100 to the surface of the target area of the subject's body comprises applying a bandage (e.g., Tegaderm™), a wrap (e.g., Coban™ wrap), and/or sterile gauze to patch device 100 (e.g., to backing component 130 and/or first surface 160 of patch device 100).

In many cases, a method of treating a subject in need thereof comprises securing patch device 100 to at least a portion of a target area of the subject's body (e.g., at least a portion of the subject's skin in need of treating). In some cases, securing patch device 100 comprises applying perpendicular pressure from the patch device to the target area and/or preventing the patch device 100 from shifting position relative to the target area. In some cases, securing patch device 100 to at least a portion of a target area of the subject's body comprises contacting the portion of the target area with adhesive 150 of patch device 100. In some cases, securing patch device 100 to the subject's body comprises applying a bandage (e.g., Tegaderm™), a wrap (e.g., Coban™ wrap), and/or sterile gauze to patch device 100 and/or to the subject's body. In some cases, securing patch device 100 to the portion of the target area of the subject's body comprises contacting the portion of the target area with adhesive 150 and applying a bandage, a wrap, and/or sterile gauze to patch device 100 and/or to the subject's body.

In some cases, a method of treating a subject in need thereof comprises applying a plurality of patch devices 100 to a target area of a subject or portion(s) thereof (e.g., to a plurality of different locations of the target area), for example, to more completely cover a target area larger than each patch device of the plurality of patch devices. In some cases, a first patch device of the plurality of patch devices is of the same composition as a second patch device of the plurality of patch devices. In some cases, methods disclosed herein comprise forming (e.g., manufacturing) a first patch device of the plurality of patch devices with a lower concentration of pigment-producing cells than a second patch device of the plurality of patch devices, for example, to better match a pigmentation gradient in the target area. In some cases, methods disclosed herein comprise forming (e.g., manufacturing) a first patch device of the plurality of patch devices with a higher concentration of pigment-producing cells than a second patch device of the plurality of patch devices, for example, to better match a pigmentation gradient in the target area. In some cases, a method of treating a subject in need thereof comprises applying a second patch device to a portion of the target area while the first patch device is applied to a different portion of the same target area. In some cases, a method of treating a subject in need thereof comprises applying a first patch device to a portion of the target area before applying a second patch device to at least some of the same portion of the target area to which the first patch device was applied. For example, some embodiments of a method of treating a subject in need thereof comprise applying a second patch device to a second portion of the target area after a first patch device is removed from a first portion of the target area, wherein the second portion of the target area comprises at least some of the first portion. In many cases, increases in treatment efficiency (e.g., increased transfer efficiency of pigment-producing cells and post-transfer viability of pigment-producing cells) resulting from improvements to the structure and composition of patch devices 100 disclosed herein obviate the need for treatment of a portion of a target area with a second patch device subsequent to treatment of the portion of the target area with a first patch device. In some embodiments, a method of treating a subject in need thereof comprises applying no more than one patch device to the target area.

In many cases, a method of treating a subject comprises transferring all or a portion of the cells comprising cellular component 110 from patch device 100 to a target area of a subject's body (e.g., a hypopigmented or depigmented portion of the subject's skin). In some cases, transferring all or a portion of the cells comprising cellular component 110 from patch device 100 to the target area comprises administering patch device 100 to the patient. For example, a method of treating a subject comprises transferring all or a portion of the cells comprising cellular component 110 from patch device 100 to a target area of a subject's body by applying patch device 100 to the target area. In some cases, applying patch device 100 to a target area of a subject comprises contacting the target area with patch device 100 (e.g., second surface 170 of the patch device and/or adhesive 150).

In some cases, methods disclosed herein comprise transferring at least a portion (e.g., at least 50%, at least 60%, at least 70%, at least 80%, at least 85%, at least 90%, at least 95%, at least 96%, at least 97%, at least 98%, at least 99%, at least 99.5%, or at least 100%) of the pigment-producing cells of cellular component 110 from patch device 100 to a target area of a subject (e.g., a hypopigmented or depigmented portion of the subject's skin) during treatment.

In some cases, transferring at least a portion of the pigment-producing cells of cellular component 110 from patch device 100 to a target area of a subject results in repigmentation of at least 50%, at least 60%, at least 70%, at least 80%, at least 85%, at least 90%, at least 95%, at least 96%, at least 97%, at least 98%, at least 99%, at least 99.5%, or 100% of the target area of the subject within 3 months, wherein the target area is an area that, prior to transfer to such area, was a hypopigmented or depigmented portion of the subject's skin.

In some cases, transferring at least a portion of the pigment-producing cells of cellular component 110 from patch device 100 to a target area of a subject results in repigmentation of at least 50%, at least 60%, at least 70%, at least 80%, at least 85%, at least 90%, at least 95%, at least 96%, at least 97%, at least 98%, at least 99%, at least 99.5%, or 100% of the target area of the subject within 6 months, wherein the target area is an area that, prior to transfer to such area, was a hypopigmented or depigmented portion of the subject's skin.

In some cases, transferring at least a portion of the pigment-producing cells of cellular component 110 from patch device 100 to a target area of a subject results in repigmentation of at least 50%, at least 60%, at least 70%, at least 80%, at least 85%, at least 90%, at least 95%, at least 96%, at least 97%, at least 98%, at least 99%, at least 99.5%, or 100% of the target area of the subject within 9 months, wherein the target area is an area that, prior to transfer to such area, was a hypopigmented or depigmented portion of the subject's skin.

In some cases, transferring at least a portion of the pigment-producing cells of cellular component 110 from patch device 100 to a target area of a subject results in repigmentation of at least 50%, at least 60%, at least 70%, at least 80%, at least 85%, at least 90%, at least 95%, at least 96%, at least 97%, at least 98%, at least 99%, at least 99.5%, or 100% of the target area of the subject within 12 months, wherein the target area is an area that, prior to transfer to such area, was a hypopigmented or depigmented portion of the subject's skin.

In many cases, a method of treating a subject comprises repigmenting a target area of a subject using a patch device 100 disclosed herein.

In some cases, methods disclosed herein comprise repigmenting at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) within 3 months following application of one or more patch devices to the target area. In some cases, transferring at least a portion of the pigment-producing cells of cellular component 110 from patch device 100 to a target area of a subject results in at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) being repigmented within 3 months following application of one or more patch devices to the target area.

In some cases, methods disclosed herein comprise repigmenting at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) within 6 months following application of one or more patch devices to the target area. In some cases, transferring at least a portion of the pigment-producing cells of cellular component 110 from patch device 100 to a target area of a subject results in at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) being repigmented within 6 months following application of one or more patch devices to the target area.

In some cases, methods disclosed herein comprise repigmenting at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) within 9 months following application of one or more patch devices to the target area. In some cases, transferring at least a portion of the pigment-producing cells of cellular component 110 from patch device 100 to a target area of a subject results in at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) being repigmented within 9 months following application of one or more patch devices to the target area.

In some cases, a methods disclosed herein comprise repigmenting at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) within 12 months following application of one or more patch devices to the target area. In some cases, transferring at least a portion of the pigment-producing cells of cellular component 110 from patch device 100 to a target area of a subject results in at least 50%, at least 60%, at least 70%, at least 80%, at least 85%, at least 90%, at least 95%, at least 96%, at least 97%, at least 98%, at least 99%, at least 99.5%, or at least 100% of the target area of a subject's skin (e.g., hypopigmented or depigmented target area) being repigmented within 12 months following application of one or more patch devices to the target area.

In some cases, treating a subject (e.g., a subject having hypopigmentation or depigmentation of the skin) using patch device 100 results in a more even distribution of pigment-producing cells at the target area than an alternative repigmentation treatment, such as treatments comprising administration of cells in a non-viscous liquid suspension (e.g., an aqueous cell suspension), a viscous suspension (e.g., a paste), using a rigid stamp, using a bandage, or using a tape (e.g., as determined using a method disclosed herein, such as reflectance spectroscopy or visual inspection). For example, a method of treating a subject comprising administering patch device 100 to a target area of the subject's skin is effective to transfer the cells of cellular component 110 (e.g., pigment-producing cells of cellular component 110) more evenly than alternative repigmentation treatments, such as treatments comprising administration of cells in a non-viscous liquid suspension (e.g., an aqueous cell suspension), a viscous suspension (e.g., a paste), using a rigid stamp, using a bandage, or using a tape (e.g., as determined using a method disclosed herein, such as reflectance spectroscopy or visual inspection).

The devices and methods of the present disclosure are useful in treating a skin condition in a subject. In particular, the devices and methods disclosed herein are useful in the treatment (e.g., repigmentation) of regions of hypopigmented or depigmented skin (e.g., target areas) of a subject. In some cases, the hypopigmentation or depigmentation of the skin of a subject is caused by a disease. For example, the devices and methods disclosed herein are useful in the treatment of a subject that has vitiligo. In some cases, a target area of a subject is hypopigmented or depigmented as a result of a wound or scar resulting from a wound. For example, a target area of a subject is hypopigmented or depigmented as a result of a burn wound (or scar resulting therefrom). In some cases, a target area comprises a diabetic wound.

In many cases, a method disclosed herein comprises evaluating repigmentation of all or a portion of a target area (e.g., after use of a patch device on the target area). In some cases, repigmentation of all or a portion of a target area is performed using a quantitative method (e.g., measurement of melanin content), semi-quantitative (e.g., scoring repigmentation based on visual inspection or image analysis), a qualitative method (e.g., visual inspection by a trained technician or physician), or a combination thereof. In many cases, a method of treating a subject in need thereof comprises determining a melanin content of a portion of the subject's skin. In some cases, determining melanin content of a portion of a subject's skin comprises determining melanin content of at least a portion of a target area of the subject's skin (e.g., a portion of a target area treated with patch device 100). In some cases, determining melanin content of a portion of a subject's skin comprises determining melanin content of a reference surface of the subject's skin (e.g., a portion of the subject's skin not treated by patch device 100 or a portion of the subject's skin not having hypopigmentation or depigmentation). In some cases, the melanin content of a reference surface of the subject's skin is substantially the same as the desired melanin content in a portion of skin (e.g., the portion of a target area of the subject's skin) treated with patch device 100. In some cases, the reference surface of the subject's skin is selected based on the melanin content of the reference surface (e.g., the similarity of the melanin content of the reference surface to a desired melanin content at a portion of the subject's skin treated with patch device 100). In some cases, the melanin content of at least a portion of a target area of the subject's skin (e.g., a portion of the target area treated with patch device 100) is compared to the melanin content of a reference surface of the subject's skin (e.g., a portion of the subject's skin not treated by patch device 100 or a portion of the subject's skin not having hypopigmentation or depigmentation), for example, to determine the effectiveness (e.g., efficiency) of repigmentation resulting from treatment of the subject with patch device 100. The melanin content may be determined through evaluation of cells, such as by spectrophotometry as melanin content per cell or melanin content per culture area. Handheld microprocessor controlled reflectance spectrophotometer may be used which provides a readout of the erythema and melanin indices as a function of the absorbance characteristic of human skin. The indices of erythema and melanin increase as the skin becomes more erythematous and more pigmented, so the melanin index (M-index) can be regarded as a parameter which is mainly influenced by the melanin content. Thus skin color and therefore melanin can be quantified by reflectance photometric techniques including tristimulus colorimetry and narrow-band spectrophotometry. Narrow band spectrophotometric devices compute melanin index (MI) as an objective measure of the skin melanin content. Other methods known in the art may alternatively be used to determine the amount of melanin in the target area.

In some cases, the melanin content of at least a portion of the subject's skin treated with patch device 100 (e.g., a portion of the target area treated with patch device 100) is 50 percent to 100 percent of the melanin content of a reference surface of the subject's skin, as measured 1 month after treatment with patch device 100. In some cases, the melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the melanin content of a reference surface of the subject's skin, as measured 1 month after treatment with patch device 100. In some cases, the melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the melanin content of a reference surface of the subject's skin, as measured 1 month after treatment with patch device 100. In some cases, the melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the melanin content of a reference surface of the subject's skin, as measured 1 month after treatment with patch device 100. In some cases, the melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the melanin content of a reference surface of the subject's skin, as measured 1 month after treatment with patch device 100.

In some cases, the melanin content of at least a portion of the subject's skin treated with patch device 100 (e.g., a portion of the target area treated with patch device 100) is 50 percent to 100 percent of the melanin content of a reference surface of the subject's skin, as measured 3 months after treatment with patch device 100. In some cases, the melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the melanin content of a reference surface of the subject's skin, as measured 3 months after treatment with patch device 100. In some cases, the melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the melanin content of a reference surface of the subject's skin, as measured 3 months after treatment with patch device 100. In some cases, the melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the melanin content of a reference surface of the subject's skin, as measured 3 months after treatment with patch device 100. In some cases, the melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the melanin content of a reference surface of the subject's skin, as measured 3 months after treatment with patch device 100.

In some cases, the melanin content of at least a portion of the subject's skin treated with patch device 100 (e.g., a portion of the target area treated with patch device 100) is 50 percent to 100 percent of the melanin content of a reference surface of the subject's skin, as measured 6 months after treatment with patch device 100. In some cases, the melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the melanin content of a reference surface of the subject's skin, as measured 6 months after treatment with patch device 100. In some cases, the melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the melanin content of a reference surface of the subject's skin, as measured 6 months after treatment with patch device 100. In some cases, the melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the melanin content of a reference surface of the subject's skin, as measured 6 months after treatment with patch device 100. In some cases, the melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the melanin content of a reference surface of the subject's skin, as measured 6 months after treatment with patch device 100.

In some cases, the melanin content of at least a portion of the subject's skin treated with patch device 100 (e.g., a portion of the target area treated with patch device 100) is 50 percent to 100 percent of the melanin content of a reference surface of the subject's skin, as measured 9 months after treatment with patch device 100. In some cases, the melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the melanin content of a reference surface of the subject's skin, as measured 9 months after treatment with patch device 100. In some cases, the melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the melanin content of a reference surface of the subject's skin, as measured 9 months after treatment with patch device 100. In some cases, the melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the melanin content of a reference surface of the subject's skin, as measured 9 months after treatment with patch device 100. In some cases, the melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the melanin content of a reference surface of the subject's skin, as measured 9 months after treatment with patch device 100.

In some cases, the melanin content of at least a portion of the subject's skin treated with patch device 100 (e.g., a portion of the target area treated with patch device 100) is 50 percent to 100 percent of the melanin content of a reference surface of the subject's skin, as measured 12 months after treatment with patch device 100. In some cases, the melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the melanin content of a reference surface of the subject's skin, as measured 12 months after treatment with patch device 100. In some cases, the melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the melanin content of a reference surface of the subject's skin, as measured 12 months after treatment with patch device 100. In some cases, the melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the melanin content of a reference surface of the subject's skin, as measured 12 months after treatment with patch device 100. In some cases, the melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the melanin content of a reference surface of the subject's skin, as measured 12 months after treatment with patch device 100.

In some cases, determining melanin content of a portion of a subject's skin comprises optical evaluation. For example, determining melanin content of a portion of a subject's skin comprises measuring light reflected by the portion of the subject's skin, in some cases. In some cases, measuring light reflected by the portion of the subject's skin comprises spectrometry. In some cases, determining melanin content comprises by diffuse reflectance spectroscopy (DRS). In some cases, determining melanin content by DRS comprises measuring one or more visible wavelengths reflected by the portion of the subject's skin. In some cases, determining melanin content comprises measuring light intensity (e.g., normalized light intensity) in one or more of the red, green, and blue wavelength ranges. For example, determining melanin content comprises measuring light intensity of reflected red light in a range from about 500 nm to about 675 nm, measuring light intensity of reflected green light in a range from about 425 nm to about 625 nm, and/or measuring light intensity of reflected blue light in a range from about 375 nm to about 550 nm. In some cases, determining melanin content comprises spectral imaging. In some cases, determining melanin content comprises the use of the spectrum resolution method.

In some cases, determining repigmentation or melanin content of a target area comprises visual inspection (e.g., as performed by a technician or physician). FIG. 14 shows the imaging of pigmentation at a target area (indicated by the dotted line) treated with a patch device, in accordance with some embodiments. For example, a technician, a physician, or a computer program (e.g., employing artificial intelligence or a classifier) is used to evaluate images of all or a portion of a target area on which a patch device, system, or method disclosed herein was used (e.g., after blinding of patient or treatment group information), in some embodiments. In some cases, the technician, physician or computer program assigns a numerical score or other semi-quantitative score measured against a standard rubric is assigned based on optical (e.g., visual) inspection of the image, and the extent of repigmentation or melanin content is determined by comparing scores of images from cell-treated or control-treated groups. In some cases, repigmentation or melanin content of at least a portion of the subject's skin treated with patch device 100 (e.g., a portion of the target area treated with patch device 100) is 50 percent to 100 percent of the repigmentation or melanin content of a reference surface of the subject's skin, as measured 12 months after treatment with patch device 100 (e.g., as determined by optical or visual inspection). In some cases, repigmentation or melanin content of at least a portion of the subject's skin treated with patch device 100 (e.g., a portion of the target area treated with patch device 100) is 50 percent to 60 percent, 50 percent to 70 percent, 50 percent to 80 percent, 50 percent to 85 percent, 50 percent to 90 percent, 50 percent to 95 percent, 50 percent to 97 percent, 50 percent to 99 percent, 50 percent to 100 percent, 60 percent to 70 percent, 60 percent to 80 percent, 60 percent to 85 percent, 60 percent to 90 percent, 60 percent to 95 percent, 60 percent to 97 percent, 60 percent to 99 percent, 60 percent to 100 percent, 70 percent to 80 percent, 70 percent to 85 percent, 70 percent to 90 percent, 70 percent to 95 percent, 70 percent to 97 percent, 70 percent to 99 percent, 70 percent to 100 percent, 80 percent to 85 percent, 80 percent to 90 percent, 80 percent to 95 percent, 80 percent to 97 percent, 80 percent to 99 percent, 80 percent to 100 percent, 85 percent to 90 percent, 85 percent to 95 percent, 85 percent to 97 percent, 85 percent to 99 percent, 85 percent to 100 percent, 90 percent to 95 percent, 90 percent to 97 percent, 90 percent to 99 percent, 90 percent to 100 percent, 95 percent to 97 percent, 95 percent to 99 percent, 95 percent to 100 percent, 97 percent to 99 percent, 97 percent to 100 percent, or 99 percent to 100 percent of the repigmentation or melanin content of a reference surface of the subject's skin, as measured 12 months after treatment with patch device 100 (e.g., as determined by optical or visual inspection). In some cases, repigmentation or melanin content of at least a portion of the subject's skin treated with patch device 100 (e.g., a portion of the target area treated with patch device 100) is 50 percent, 60 percent, 70 percent, 80 percent, 85 percent, 90 percent, 95 percent, 97 percent, 99 percent, or 100 percent of the repigmentation or melanin content of a reference surface of the subject's skin, as measured 12 months after treatment with patch device 100 (e.g., as determined by optical or visual inspection). In some cases, repigmentation or melanin content of at least a portion of the subject's skin treated with patch device 100 (e.g., a portion of the target area treated with patch device 100) is at least 50 percent, 60 percent, 70 percent, 80 percent, 85 percent, 90 percent, 95 percent, 97 percent, or 99 percent of the repigmentation or melanin content of a reference surface of the subject's skin, as measured 12 months after treatment with patch device 100 (e.g., as determined by optical or visual inspection). In some cases, repigmentation or melanin content of at least a portion of the subject's skin treated with patch device 100 (e.g., a portion of the target area treated with patch device 100) is at most 60 percent, 70 percent, 80 percent, 85 percent, 90 percent, 95 percent, 97 percent, 99 percent, or 100 percent of the repigmentation or melanin content of a reference surface of the subject's skin, as measured 12 months after treatment with patch device 100 (e.g., as determined by optical or visual inspection).

In some cases, measuring light reflected by the portion of the subject's skin comprises determining a melanin index. In some cases, determining a melanin index comprises measuring the reflectance of light from a portion of a subject's skin at one or more wavelength (e.g., 905 nm and/or 635 nm) or ranges of wavelengths. In some cases, determining a melanin index comprises calculating a melanin index (MI) using the formula: MI=1000×log 10(R905 nm/R632 nm), wherein R905 nm and R632 nm represent the reflectance measured at 905 nm and at 632 nm, respectively. In some cases, measuring light reflected by the portion of the subject's skin comprises determining a hemoglobin index. In some cases, determining a melanin index comprises calculating an erythema index (EI) using the formula: EI=1000×log 10(R632 nm/R546 nm), wherein R632 nm and R546 nm represent the reflectance measured at 632 nm and at 546 nm, respectively. In some cases, determining a melanin content of a portion of a subject's skin comprises measuring light reflected by the portion of the subject's skin at certain wavelengths and/or in certain ranges of wavelengths (e.g., using a spectrometer). For example, determining a melanin content of a portion of a subject's skin comprises determining a slope of the absorbance spectrum within a range of wavelengths of light (e.g., from about 620 nm to about 720 nm in determining melanin concentration or from about 560 nm to about 580 nm in determining hemoglobin content).

In some cases, determining melanin content comprises using the ‘tristimulus’ measurement system. In some cases, determining melanin content using the ‘tristimulus’ measurement system comprises measuring light intensity from black to white (e.g., on a 0 to 100 scale, wherein 0 represents black) reflected from a portion of a subject's skin, measuring light intensity from green to red (e.g., on a scale in which green values are negative numbers, red values are positive numbers, and the scale crosses the black to white scale at a green-to-red value of 0) reflected from the portion of the subject's skin, and measuring light intensity from blue to yellow (e.g., on a scale in which blue values are negative numbers, yellow values are positive numbers, and the scale crosses the black to white scale at a blue-to-yellow values of 0). In some cases, melanin content is determined using the following formula: melanin content=180/p×(arctan(((black-to-white scale numerical value)−50)/(blue-to-yellow scale numerical value).

In some cases, determining melanin content of a portion of a subject's skin comprises an objective visual evaluation of the pigmentation of the portion of the subject's skin (e.g., by a technical analyst or a medical professional). In some cases, determining melanin content of a portion of a subject's skin comprises a subjective visual evaluation of the pigmentation of the portion of the subject's skin (e.g., by a technical analyst or a medical professional).

In many cases, the subject is a mammal. The subject is a human (e.g., a human patient) in most cases. In some cases, the methods and devices disclosed herein are also useful in treating a skin condition (e.g., hypopigmentation or depigmentation) in non-human mammals, such as a dog, a cat, a horse, a non-human primate, a pig, a cow, a rat, a mouse, a goat, or a rabbit.

A method of treating a subject comprises identifying a subject (e.g., a patient) having a skin condition comprising hypopigmentation or depigmentation at a target area of the subject's skin. In some cases, the skin condition comprising hypopigmentation or depigmentation is vitiligo. In some cases, the skin condition comprising hypopigmentation or depigmentation is scarring of the skin. In some cases, identifying a subject having a skin condition comprising hypopigmentation or depigmentation comprises evaluating the patient medically. In some cases, a subject (e.g., a patient) has been previously diagnosed with a skin condition comprising hypopigmentation or depigmentation, such as vitiligo. In some cases, identifying a subject having a skin condition comprising hypopigmentation or depigmentation comprises determining that the subject (e.g., a patient) has a family history of the skin condition, such as vitiligo. In some cases, identifying a subject having a skin condition comprising hypopigmentation or depigmentation comprises determining that the subject (e.g., a patient) has experienced a trigger event (e.g., sunburn, stress, or exposure to industrial chemicals) known to cause the skin condition, such as vitiligo.

In some cases, a method of treating a subject comprises selecting a concentration or quantity of pigment-producing cells disclosed herein for fabrication of patch device 100 based on a pigmentation of the target area of the subject's skin to be treated with the patch device or on a pigmentation of an area adjacent to the target area of the subject's skin to be treated with the patch device. The melanocytes types or quantities, or concentrations thereof may also incorporate an evaluation and determination of the types of melanin that the melanocytes produce, in order to result in a skin tone that is adapted to the individual subject's own unique skin tone, unless the subjects' own melanocytes are used. When a subjects' own melanocytes are used, the skin tone resulting will necessary align substantially for the subject. Skin tone, generally speaking, is not based only on melanocyte concentration. Rather, skin tone is based on the ratio of the types of melanin that melanocytes produce. Enough melanocytes are necessary to produce the subjects' natural skin tone, but the concentration of melanocytes is generally similar between people with different race/skin tones. Thus, use of melanocytes of the resulting target skin tone are necessary in subjects wishing to match their natural skin tone, and at sufficient quantity and concentration to achieve such result, as noted herein.

### IV. Definitions

The terminology used herein is for the purpose of describing particular cases only and is not intended to be limiting. As used herein, the singular forms “a”, “an” and “the” are intended to include the plural forms as well, unless the context clearly indicates otherwise When used in the following claims, the terms “comprise”, “include”, “have” and their conjugates mean “including but not limited to.” Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”. Furthermore, to the extent that the terms “including”, “includes”, “having”, “has”, “with”, or variants thereof are used in either the detailed description and/or the claims, such terms are intended to be inclusive in a manner similar to the term “comprising”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The term “about” or “approximately” means within an acceptable error range for the particular value as determined by one of ordinary skill in the art, which will depend in part on how the value is measured or determined, e.g., the limitations of the measurement system. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

Further, as used herein in the specification and claims, including as used in the examples and unless otherwise expressly specified, all numbers may be read as if prefaced by the word “about” or “approximately.” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring.” “evaluating,” “assessing,” “assaying.” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” refer to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be suspected as having, or of being at high risk for developing, a disease or condition. In some cases, the subject is not necessarily diagnosed or suspected of being at high risk for the disease.

In some cases, the subject is a “patient” that has been diagnosed with the disease or condition. The terms “individual.” “patient,” or “subject” are used interchangeably. None of the terms require or are limited to situation characterized by the supervision (e.g. constant or intermittent) of a health care worker (e.g, a doctor, a registered nurse, a nurse practitioner, a physician's assistant, an orderly, or a hospice worker).

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

### Example 1

### Isolation of Human Melanocytes for Patch Device

This example shows methods for isolating primary human melanocytes for use in patch devices and methods disclosed herein.

Human skin samples were collected from skin reduction surgeries and shipped from the clinical collection site to the laboratory overnight on ice in Dulbecco's Modified Eagle's Medium (DMEM) with 1% penicillin and 1% streptomycin (e.g., 1% pen/strep) and 1% amphotericin under sterile conditions. Each skin sample was cut into 1 cm² pieces after receipt at the laboratory, soaked in 70% ethanol for 1 minute and then washed twice with PBS without calcium or magnesium. Each piece of skin was incubated in 5 mL of digestion medium overnight at 37° C. Skin samples can also be digested overnight at 4° C., for example, if a digestion less strenuous on cell viability is desired. Digestion medium consisted of 2.4 U/mL of dispase (Zen-Bio, Inc.) in melanocyte growth medium (PromoCell® Melanocyte Growth Medium M2 (MGM M2)). If melanocyte growth medium is unavailable, skin samples can be digested in 5 mL of digestion medium consisting of 2.4 U/mL dispase in phosphate-buffered saline (PBS) overnight at either 4° C., or 37° C. Digestion medium consisting of 0.25% trypsin and 0.1% EDTA in PBS can be used to digest cut skin samples for 30 minutes to 85 minutes at 37° C.

TABLE 1

| | | Sample | # Cells/ | | | | Melanocytes/ |
|---|---|---|---|---|---|---|---|
| Harvest | | Area | cm² of | Viability | Melanocytes | Keratinocytes | cm² of |
| conditions | # of cells | (cm²) | Sample | (%) | (%) | (%) | Sample |
| DermaBlade, 15 | 14.6 × 10⁶ | 7.7 | 1896104 | 90.7 | 17.7 | 50.3 | 335610 |
| min trypsin | | | | | | | |
| DermaBlade, 45 | 2.82 × 10⁶ | 15.3 | 1843137 | 87.6 | 15.7 | 63.0 | 289373 |
| min trypsin | | | | | | | |
| Dispase PBS, | 6.00 × 10⁵ | 10 | 60000 | 20.7 | — | — | — |
| overnight 37° C. | | | | | | | |
| Dispase MGMM2, | 4.90 × 10⁶ | 10 | 490000 | 75.0 | 24.3 | 62.4 | 119070 |
| overnight 37° C. | | | | | | | |
| Dispase PBS, | 1.70 × 10⁶ | 12 | 141667 | 85.0 | — | — | — |
| overnight 4° C. | | | | | | | |
| Dispase MGMM2, | 7.40 × 10⁶ | 12 | 616667 | 80.0 | 22.1 | 53.2 | 136283 |
| overnight 4° C. | | | | | | | |

It was found that the epidermis can be peeled from the dermis of the skin samples using forceps after incubation in digestion medium most easily if digestion medium consisting of dispase and MGMM2 is used and the skin samples are placed in incubation vessels with the epidermis oriented downward. If difficulty in removing the epidermis from the dermis is experienced after initial incubation in digestion medium, samples can be incubated for up to 3 additional hours at room temperature in digestion medium consisting of 2.4 U/mL dispase in MGMM2, with the epidermis of the sample oriented downward. Isolated epidermis samples were subsequently subjected to cell isolation, as described below.

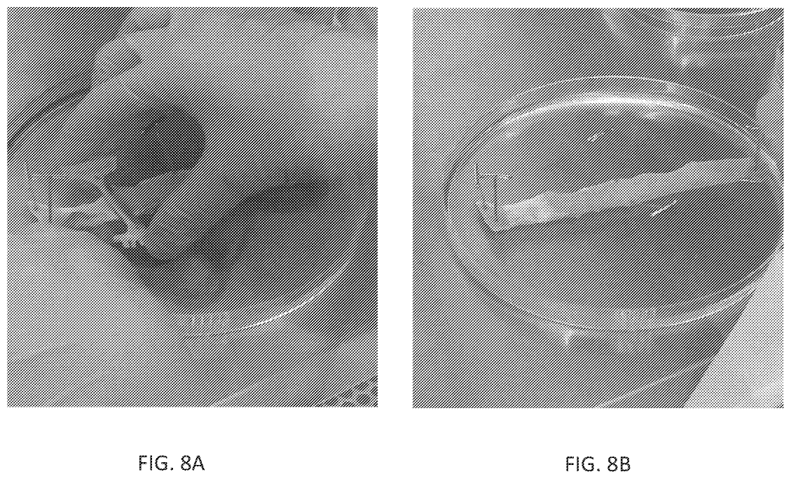

Alternately, a DermaBlade® shave biopsy instrument can be used to remove the epidermis of cut skin samples from the dermis, for example, if a sample from a particular patient or skin harvest location does not result in ideal epidermis removal. The shave biopsy instrument is passed over the cut skin sample to remove the epidermis, for example, as shown in FIGS. 8A and 8B. Scraped epidermis samples were subsequently subjected to cell isolation, as described below. Because scraping the epidermis off of the dermis using a shave biopsy instrument can result in the removal of portions of the dermis at the same time, isolated epidermal cells can be purified from isolated dermal cells prior to or subsequent to culturing, for example, using fluorescence-assisted cell sorting (FACS) or magnetic-activated cell sorting (MACS), if so desired. For example, a population of melanocytes (e.g., CD45-negative. CD49f-low, and cKit-positive cells) may be isolated or enriched, for example using FACS or MACS, for use in the cellular component of a patch device described herein. A population of keratinocytes (e.g., CD45-negative, CD49f-high cells) may be isolated or enriched, for example using FACS or MACS, for use in the cellular component of a patch device.

Following removal of the epidermis from the dermis, the epidermis was cut into smaller pieces (1 mm²-3 mm² in area) using a scalpel. Cells were isolated from the epidermis by incubating the samples in a medium comprising 0.25% trypsin and 0.1% EDTA in PBS for 15 minutes to 45 minutes at 37° C. Samples were mechanically agitated by aspirating up and down using a serological pipette to free additional cells. An equal volume of soybean trypsin inhibitor was added to neutralize the trypsin, and the resulting cell suspension was centrifuged at 450×g for 15 minutes. Tissues found to be floating in the centrifuged samples were removed using forceps, and the supernatant liquid was aspirated off of the cell pellet. Cell pellets were resuspended in MGMM2 medium, and cell viability was assessed by counting cells using trypan blue and a hemacytometer. Resuspended cells were plated at a density of 1.25×10⁵ live cells (as determined by trypan blue exclusion) in T25 culture flasks. Results of melanocyte isolation methods are shown in Table 1. Optionally, fibroblasts are eliminated from cell cultures by culturing the plated cells in basal media including 1% antibiotic/antimycotic (AA) and 150 μM geneticin for four days (e.g., days 4-7 following initial plating).

### Example 2

### Expansion of Human Melanocytes for Patch Device

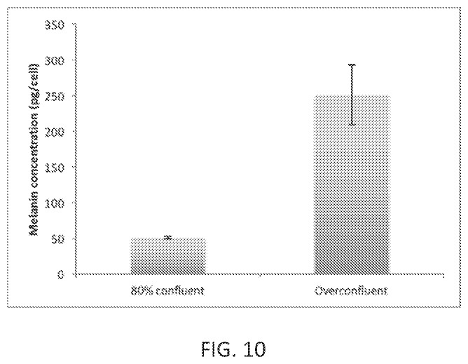

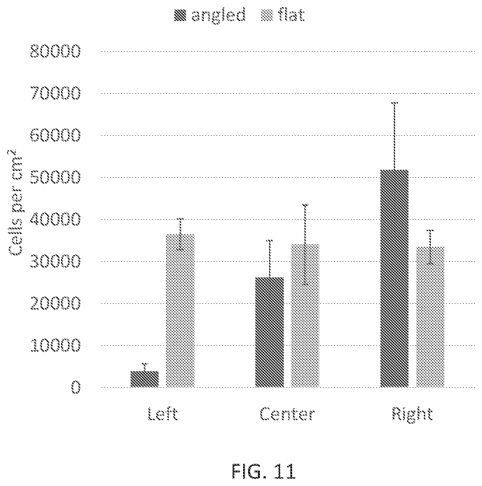

This example shows a method for expanding primary human melanocytes for use in patch devices and methods disclosed herein. Melanin concentration in cultured melanocytes was improved by allowing cultured melanocytes to exceed confluency in culture (see. FIG. 10).

Human melanocytes to be used in patch devices and related methods described herein were expanded by culturing under standard culture conditions of 37° C. and 5% CO₂ in a standard tissue incubator. Basal cell culture media (MGMM2), including 1% AA, was used to feed the cells two times per week.

After 11 days in culture, cells were passaged using differential trypsinization. Cells were passaged at approximately 70-80% confluence using differential trypsinization. Differential trypsinization involved incubating cultured cells with 0.05% trypsin every 2 minutes for 10 minutes followed by an additional incubation with 0.05% trypsin for 10 minutes. Soybean trypsin inhibitor was added to neutralize the trypsin each time it was removed at the end of each incubation period. Cells were removed for subculture at the end of each incubation period. In additional experiments, cells were passaged at 70-80% confluence using traditional one-step trypsinization wherein all cells are lifted and replated together. Subcultured cell cultures showed high melanocyte concentrations and low keratinocyte concentrations. FIG. 3 shows immunostaining results of primary human melanocyte cultures, in accordance with embodiments (scale bar indicates 1 mm, arrows indicate cells staining positive for K-14, positive staining without arrows indicates cells staining positive for TRP-1, asterisks indicate nuclear staining (DAPI) of cells not staining positively for K-14 or TRP-1 (which cells may include immune cells. Merkel cells, Langerhans, and/or fibroblasts)) showing few cells staining positive for K-14 expression (arrows, indicating keratinocyte phenotype) and many cells staining positive for TRP-1 (positive staining without arrows, indicating melanocyte phenotype).

Three different basal media were tested for melanocyte culture: PromoCell® Melanocyte Growth Medium M2 (MGMM2), Medium 254 (M254, ThermoFisher Scientific) supplemented with Human Melanocyte Growth Supplement-1 (HMGS-1, ThermoFisher Scientific), and M254 supplemented with Human Melanocyte Growth Supplement-2 (HMGS-2, ThermoFisher Scientific). Results of culture media tests showed that MGMM2 medium maintained better melanocyte morphology (e.g., more stellate and/or less rounded) and elicited higher melanocyte proliferation rates. MGMM2 culture medium also reflected fewer keratinocytes in culture over time than M254 with HMGS-1. Results of culture media tests also showed that M254 with HMGS-2 showed improved melanocyte proliferation rates and selection for melanocyte cells. These and other data from culture media experiments are shown in Table 2.

TABLE 2

| | | | Initial # | | | | | | |
|---|---|---|---|---|---|---|---|---|---|
| | | | of | Days | | | | Number of | (Mfinal − |
| | | Initial # of | Melano- | Between | Cells at | Melanocytes | Keratinocytes | Melanocytes | Minitial)/ |
| | Media | Cells | cytes | Passage | passage | at passage (%) | at passage (%) | at passage | Minitial |
| DermaBlade, | HMGS-2 | 3.125 × 10⁶ | 4.91 × 10⁵ | 17 | 1.11 × 10⁶ | 99.6 | 0.0 | 1.1 × 10⁶ | 6.51 |
| 45 min Trypsin | | | | | | | | | |
| DermaBlade, | PM2 | 3.125 × 10⁶ | 4.91 × 10⁵ | 21 | 1.60 × 10⁵ | 68.1 | 5.4 | 1.09 × 10⁵ | −0.259 |
| 45 min Trypsin | | | | | | | | | |
| Dispase | HMGS-2 | 3.125 × 10⁶ | 7.59 × 10⁵ | 21 | 1.88 × 10⁵ | 92.2 | 2.8 | 1.7 × 10⁵ | −0.241 |
| MGMM2, | | | | | | | | | |
| overnight 37° C. | | | | | | | | | |
| Dispase | HMGS-2 | 3.125 × 10⁶ | 6.91 × 10⁵ | 21 | 3.80 × 10⁵ | 99.9 | 0.0 | 3.80 × 10⁵ | 0.832 |
| MGMM2, | | | | | | | | | |
| overnight 4° C. | | | | | | | | | |
| Dispase | PM2 | 3.125 × 10⁶ | 6.91 × 10⁵ | 21 | 2.95 × 10⁵ | 25.7 | 64.6 | 7.58 × 10⁵ | −0.634 |
| MGMM2, | | | | | | | | | |
| overnight 4° C. | | | | | | | | | |

### Example 3

### Fabrication of a Cell-Seeded Gel

This example shows a method for fabricating a gel comprising primary human melanocytes for use in patch devices and methods disclosed herein.

Cultured primary human melanocytes isolated as described in Example 1 were enzymatically lifted from culture and resuspended at 4 million cells/mL in M254 with HMGS-2 medium to form a cell solution for use as the cellular component of patch devices. Fifty μL of bovine fibrinogen (Sigma, 40 mg/mL in PBS, sterile filtered) solution was combined with 50 μL of cell suspension solution and 100 mL of bovine thrombin (Sigma, 4 U/mL diluted in PBS) in centrifuge tubes and the solution was mixed with a pipette for use in forming the gel substrate of patch devices. Final formulation of the mixture was 10 mg/mL fibrin, 2 U/mL thrombin. Two hundred microliters of the mixture was deposited into ten 2% agarose molds having 1 cm wide×1 cm long×2 mm high voids with flat bottoms and allowed to set at room temperature for about 30 minutes in a closed petri dish.

### Example 4

### Fabrication of a Patch Device

This example shows a method for fabricating a patch device for applying cells to a subject.

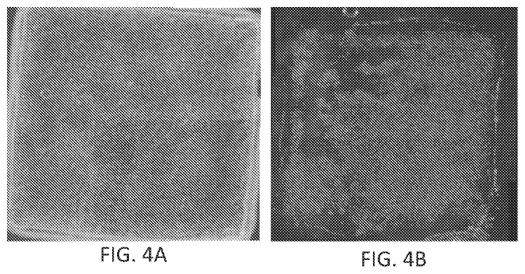

A silicone dressing (Adaptic Touch™ Silicone Non-Adhering Dressing) was placed on the top surface (e.g., the first surface) of gel substrates formed as described in Example 3 while the gel substrates were still in the agarose mold. Optionally, 20 mL of PBS was added around the agarose and the petri dish was sealed with Parafilm and stored overnight at 4° C. Patch devices were removed from the agarose mold using forceps to gently pull on the silicone dressing. An adhesive comprising 50 μL of 5 mg/mL fibrin glue mixed with 2 U/mL thrombin was applied to the second surface of the gel substrate (e.g., the bottom surface of the rectangularly-shaped gel substrate, opposite the first surface). Calcein staining of patch devices seeded with human primary melanocytes showed very good uniformity of cells across the length and width of the second surface of the patch devices (see, FIG. 4A).

### Example 5

### Additional Approach for Fabrication of a Patch Device

This example shows a method for fabricating a patch device for treating a subject as described herein.

Epidermal cells were isolated from split thickness skin obtained from surgical discard removed from a donor subject one day prior to isolation. Split thickness skin samples were shipped overnight to the lab for isolation in DMEM culture medium supplemented with 1% penicillin-streptomycin-Amphotericin B (Thermo Fisher Scientific). Cells were isolated by sectioning the tissue into pieces, disinfecting the sectioned tissue samples using a 1% antibiotic/antimycotic solution, incubating in 0.25% trypsin for 15 minutes, neutralizing trypsin activity with soybean trypsin inhibitor, aspirating the tissue with a syringe to mechanically disrupt the tissue, filtering through 70 μm filter, and centrifuging to separate the cells from the supernatant.

Cells were plated at 125,000 cells/cm² in T75 culture flasks in M254+HMGS2 culture medium (Thermo Fisher Scientific). Medium was changed four days after initial plating and every 2-3 days thereafter. After 11 days, cells were collected every 2 minutes using differential trypsinization with 0.05% trypsin at room temperature (RT). Cells were separately resuspended and plated in M254+HMGS2 culture medium at approximately 10,000 cells/cm². After 4 days, cells cultured from the 2 minute and 4 minute trypsinizations were resuspended at 1 million cells/mL in M254+HMGS2 culture medium (Passage 1 cells) and combined based on stellate morphology (assessed with respect to typical melanocyte stellate morphology in culture) before being added to the patch device.

50 μL of bovine fibrinogen solution (Sigma, 40 mg/mL diluted in phosphate-buffered saline (PBS), sterile filtered) was combined with 100 μL of bovine thrombin (Sigma, 4 U/mL diluted in PBS) in silicone molds pre-wetted with culture medium. The fibrinogen and thrombin mixture was allowed to set at room temperature for 30 minutes in a closed petri dish, 50 μL of cell suspension (prepared as described above) was added to the top (e.g., the first surface of the gel substrate) after the fibrinogen and thrombin mixture was added to the molds. Optionally, human fibrinogen and/or human thrombin are used in place of bovine fibrinogen and bovine thrombin.

Optionally, 50 μL of bovine fibrinogen solution (Sigma, 40 mg/mL diluted in phosphate-buffered saline (PBS), sterile filtered) was combined with 50 μL of cell suspension (prepared as described above) and 100 μL of bovine thrombin (Sigma, 4 U/mL diluted in PBS) in microcentrifuge tubes. The combination was mixed with a 1 mL micropipette. The final formulation of the mixture was 10 mg/mL fibrin, 2 U/mL thrombin, 200 μL of the mixture was deposited into ten 2% agarose molds (each mold measuring 1 cm×1 cm×2 mm high and having a flat bottom formed by a glass coverslip). Each agarose mold was UV sterilized for 30 minutes prior to the addition of the 200 μL mixture of cell suspension, fibrin, and thrombin.

A silicone dressing (Adaptic Touch Non-Adhering Silicone dressing mesh) was gently pressed onto the first surface of the gel substrate of the patch device while the patch was still in the agarose mold to serve as a backing component. Optionally, a fibrin-based backing component fabricated by mixing 20 mg/mL fibrinogen with 10 U/mL thrombin was used in place of silicone dressing, 20 mL of PBS was added around the agarose. The petri dish containing the patch devices and agarose molds was sealed with Parafilm and stored at 4 degrees Celsius for overnight incubation. After overnight incubation at 4 degrees Celsius (approximately 8-10 hours) patch devices were removed from the molds while holding the backing component with forceps.

A droplet of adhesive comprising 10 μL to 50 μL of 5 mg/mL fibrinogen and 2 U/mL thrombin) was applied to the second surface of the gel substrate of the patch devices. In some cases, the adhesive further comprised 0.5 mg/mL, 1.0 mg/mL, or 1.5 mg/mL hyaluronic acid to improve the adhesive's ability to retain the patch device in the position it is placed relative to the petri dish plastic while retaining the ability of the adhesive to be spread over the second surface of the gel substrate.

Efficacy of melanocyte transfer was tested by applying the patch devices to petri dishes with the adhesive-side of the patch devices oriented down, in contact with the petri dish plastic. Squares of 2% agarose gel were placed on top of the patch devices (e.g., in contact with the silicone dressing or the first surface of the gel substrate of the patch devices) to press each patch device perpendicular to the petri dish plastic. After 15 minutes of pressure. M254+HMGS2 culture medium was added to the petri dish. After approximately 24 hours of pressure, the agarose gel weights were removed from the patch devices by gently peeling the backing component off of the patch device. Optionally, the backing component is removed from the patch device after 24 hours of pressure. Culture medium was changed every 2-3 days with fresh M254+HMGS2 culture medium. Patch devices were removed from the petri dishes, and transferred cells were allowed to pigment for 2 weeks after transfer. Transferred cells were stained with calcein AM and imaged for viability and distribution using fluorescence imaging and for pigmentation and distribution using light microscopy. Cells were present in an x-y plane of the patch device at the level of the second surface of the gel substrate at a distribution density of 100,000 cells/cm². Results showed excellent transfer efficiency, viability of transferred cells, and pigmentation of transferred melanocytes. In experiments, successful transfer efficiency three days after application of the patch devices to the petri dish. Pigmentation of cells transferred to petri dishes was observed as early as seven days after patch devices were applied to the petri dish plastic. Melanocytes were found to be evenly distributed across the area contacted by the patch device at a density of approximately 100,000 cells/cm².

### Example 6

### In Vitro Transfer of Melanocytes from a Patch Device

This example shows transfer of primary human melanocytes for use in patch devices and methods disclosed herein.

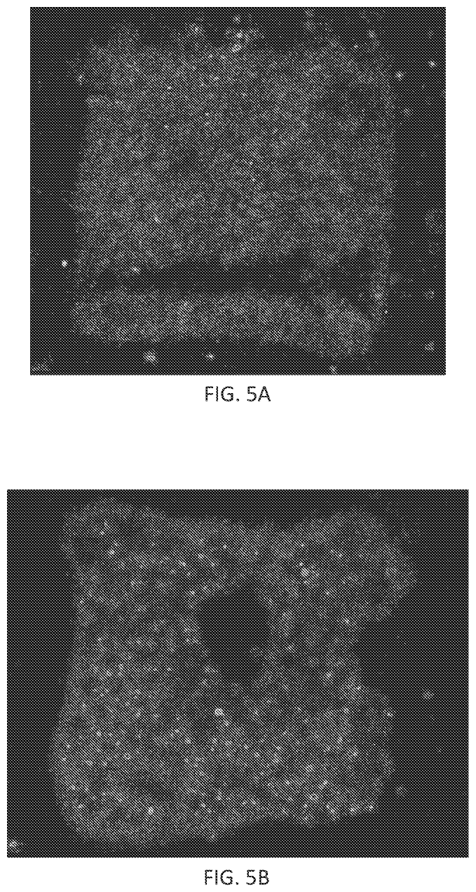

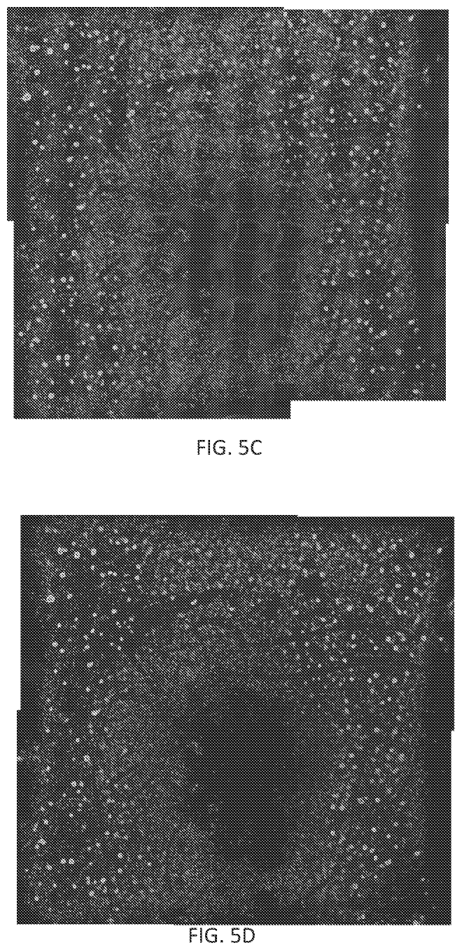

In a first instance, the melanocytes were provided pre-purified (not isolated from skin as described in Example 1), however, the methods herein may be applied to either pre-purified or melanocytes isolated from skin, as noted below. Patch devices comprising culture-expanded human melanocytes were prepared according to methods described in Examples 2-4, or Example 5. Patch devices were applied to either a plastic petri dish surface or to a decellularized dermis matrix by manipulating the patch devices using forceps on the silicone dressing backing component of each patch device. The second surface of the patch devices were applied to either the petri dish or the decellularized dermis matrix, and a square of agarose gel was placed on top of the patch devices to provide a force normal to the patch device and target area on which the patch devices were placed. After 15 minutes, M254 with HMGS-2 medium was added and the patch devices were cultured at 37° C. The agarose weights were removed the next day, and medium was changed every 2-3 days. Calcein staining showed uniform coverage of melanocytes 4 days after transfer (see, FIGS. 5A and 5B). Melanin production in transferred cells was found to be strong. In this example, decellularized dermis was used for research purposes to better mimic a skin surface. Discarded surgical skin tissue was processed to separate the dermis from the epidermis and to remove the cells from the dermis. Dispase and Triton X-100/EDTA solution were used in order to separate the dermis from epidermis and to remove the cells from the dermis, respectively.

Patch devices comprising gel substrates formed using 10 mg/mL fibrinogen and 2 U/mL thrombin were seeded with pre-purified melanocytes before a silicone mesh backing component was applied to the patch device, and patch devices were allowed to incubate overnight. Then, patch devices were used to transfer seeded melanocytes to a target area on a plastic test surface. Five days after transfer, the patch devices and transferred cells were observed and imaged (see, FIGS. 5C and 5D). The gel substrate of the patch devices had degraded by the day five time point, but melanocytes remained adherent to the target area and maintained good distribution across the target area, as determined by calcein staining of transferred cells (see, FIG. 5C). The silicone mesh was still lightly adhered to the target area but was easy to peel away. Peeling away the silicone mesh from the target area at the day five time point had a minimal impact on observed cell density and distribution across the target area, as determined by calcein staining of transferred cells on the target area (see. FIG. 5D).

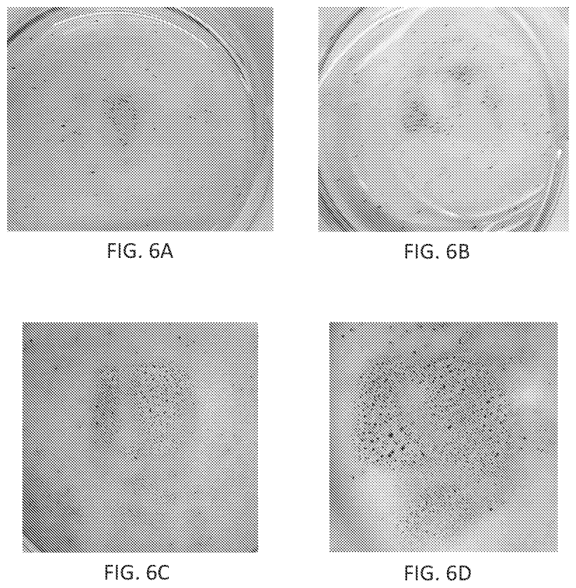

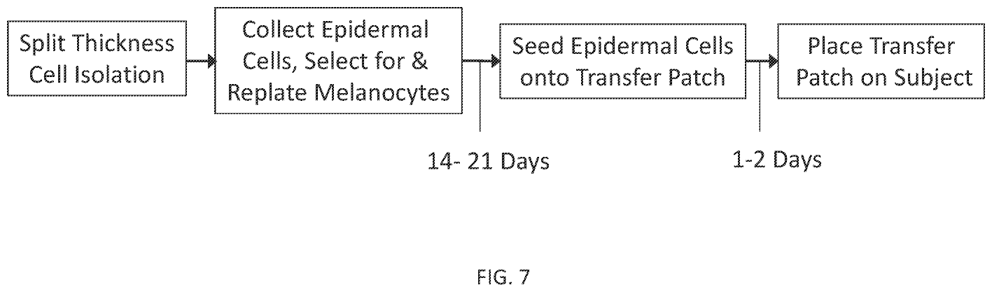

In a second instance, the melanocytes were from melanocytes isolated from skin according to Example 1, at least. Patch devices comprising culture-expanded human primary melanocytes were prepared according to methods described in Examples 1-4 or Example 5. A diagram of general workflow in patch device fabrication and use can be found in FIG. 7. Patch devices were applied to either a plastic petri dish surface or to a decellularized dermis matrix by manipulating the patch devices using forceps on the silicone dressing backing component of each patch device. The second surface of the patch devices were applied to either the petri dish or the decellularized dermis matrix, and a square of agarose gel was placed on top of the patch devices to provide a force normal to the patch device and target area on which the patch devices were placed. After 15 minutes, M254 with HMGS-2 medium was added and the patch devices were cultured at 37° C. The agarose weights were removed the next day, and medium was changed every 2-3 days. Calcein staining showed uniform coverage of melanocytes 4 days after transfer. Melanin production in transferred cells was found to be strong; some samples were evaluated qualitatively using a digital camera at seven and ten days after transfer (see, 7 days: FIGS. 6A and 6B; 10 days: FIGS. 6C and 6D, respectively).

### Example 7

### Treating a Subject Having Loss of Skin Color with a Patch Device

This example shows a method for treatment of a subject having vitiligo using a patch device comprising primary human melanocytes.

Patch devices comprising culture-expanded human primary melanocytes from a subject having vitiligo are prepared according to methods similar to those disclosed in Examples 1-4 or Example 5. Patch devices are chilled to 4° C. for transport to the clinic. The target tissue area is debrided, and patch devices are applied to the target tissue area by contacting the target tissue with the second surface of each patch device (e.g., the surface of the patch device on which adhesive was applied) until the target tissue area is covered. During the application of patch devices to the target area, forceps are used to manipulate the patch devices into position by the silicone dressing backing component before the target tissue is actually contacted by the patch devices. Silicone dressing backing components are removed from the applied patch devices without disturbing the seating of the patch devices on the target tissue area using forceps. Tegaderm bandages are used to cover and secure the patch devices in place, which allows gentle pressure to be applied to the patch devices normal to the target tissue.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A cellularized patch device, comprising:
a gel substrate having a first surface and a second surface;
a cellular component disposed within the gel substrate, the cellular component comprising a population of cells,
wherein at least 80 percent of the population of cells is disposed within a distance from the second surface of no more than 50 percent of the height of the gel substrate.

2. The device of claim 1, wherein at least 80 percent of the population of cells is disposed within a distance from the second surface of no more than 30 percent of the height of the gel substrate.

3. The device of claim 1, wherein at least 80 percent of the population of cells is disposed within a distance from the second surface of no more than 10 percent of the height of the gel substrate.

4. A cellularized patch device, comprising:
a gel substrate having a first surface and a second surface, and comprising from 10 mg/mL to 15 mg/mL fibrin and 2 U/mL thrombin;
a cellular component comprising a population of cells, the population of cells comprising a plurality of human melanocytes and wherein the population of cells is disposed within the gel substrate; and
an adhesive.

5. The device of any one of claims 1-4, wherein the population of cells is at least 90% primary human melanocytes.

6. The device of any one of claims 1-5, wherein a concentration of human melanocytes in the device is from 50,000 cells/cm² to 350,000 cells/cm².

7. The device of any one of claims 1-6, wherein the human melanocytes are applied to the first surface of the gel substrate during gel substrate formation.

8. The device of any one of claims 1-7, wherein the gel substrate comprises 10 mg/mL fibrin.

9. The device of any one of claims 1-3, further comprising an adhesive.

10. The device of any one of claims 4-9, wherein the adhesive is applied to the second surface of the gel substrate during gel substrate formation.

11. The device of any one of claims 4-10, wherein the adhesive comprises thrombin.

12. The device of claim 11, wherein a concentration of the thrombin in the adhesive is from 1 U/mL to 10 U/mL.

13. The device of claim 11 or claim 12, wherein the concentration of the thrombin in the adhesive is 2 U/mL.

14. The device of any one of claims 4-13, wherein the adhesive comprises fibrin.

15. The device of claim 14, wherein a concentration of the fibrin in the adhesive is 5 mg/mL.

16. The device of any one of claims 1-15, wherein the adhesive further comprises hyaluronic acid.

17. The device of claim 16, wherein a concentration of the hyaluronic acid in the adhesive is 1 mg/mL.

18. The device of any one of claims 1-17, further comprising a backing component.

19. The device of claim 18, wherein the backing component is coupled to the first surface of the gel substrate.

20. The device of any one of claims 18-19, wherein the backing component is flexible.

21. The device of any one of claims 18-20, wherein the backing component comprises a fibrin cap.

22. The device of claim 21, wherein the fibrin cap comprises fibrin.

23. The device of any one of claims 21-22, wherein the fibrin cap comprises at least 15 mg/mL of fibrin.

24. The device of any one of claims 21-23, wherein the fibrin cap comprises thrombin.

25. The device of any one of claims 21-24, wherein the fibrin cap comprises from 1 U/mL to 10 U/mL thrombin.

26. The device of any one of claims 21-25, wherein the fibrin cap comprises 2 U/mL of thrombin.

27. The device of any one of claims 21-26, wherein the fibrin cap comprises hyaluronic acid.

28. The device of any one of claims 21-27, wherein the fibrin cap comprises from 0.5 mg/mL to 1.5 mg/mL hyaluronic acid.

29. The device of any one of claims 18-28, wherein the backing component comprises a silicone dressing.

30. A method of fabricating a cellularized patch device, comprising:
(i) isolating a plurality of primary human melanocytes;
(ii) mixing isolated primary human melanocytes with fibrinogen and thrombin to obtain a mixture capable of forming a gel substrate, wherein concentration of the fibrinogen in the mixture is from 10 mg/mL to 15 mg/mL and concentration of the thrombin in the mixture is 2 U/mL; and
(iii) forming the gel substrate having a first surface and a second surface.

31. The method of claim 30, further comprising incubating the gel substrate for 20-30 minutes at room temperature after the mixing step.

32. The method of claim 31, wherein the gel substrate is placed in a mold during the incubating step.

33. The method of any one of claims 30-32, further comprising applying a 10 μL to 50 μL droplet of an adhesive to the second surface, wherein the adhesive comprises thrombin.

34. The method of claim 33, wherein the droplet has a volume of 50 μL.

35. The method of claim 33, wherein the droplet has a volume of 10 μL.

36. The method of any one of claims 33-35, wherein the concentration of the thrombin in the adhesive is from 1 U/mL to 10 U/mL.

37. The method of claim 35 or claim 36, wherein the concentration of the thrombin in the adhesive is 2 U/mL.

38. The method of any one of claims 35-37, wherein the adhesive comprises fibrin.

39. The method of claim 38, wherein the concentration of the fibrin in the adhesive is 5 mg/mL.

40. The method of any one of claims 38-39, wherein the adhesive comprises hyaluronic acid.

41. The method of claim 40, wherein the concentration of the hyaluronic acid in the adhesive is 1 mg/mL

42. The method of any one of claims 30-41, further comprising providing a backing component.

43. The method of claim 42, further comprising coupling the backing component to the first surface of the gel substrate.

44. The method of any one of claims 42-43, wherein the backing component is flexible.

45. The method of any one of claims 42-44, wherein the backing component comprises a fibrin cap.

46. The method of claim 45, wherein the fibrin cap comprises fibrin.

47. The method of any one of claims 45-46, wherein the fibrin cap comprises at least 15 mg/mL of fibrin.

48. The method of any one of claims 45-47, wherein the fibrin cap comprises thrombin.

49. The method of any one of claims 45-48, wherein the fibrin cap comprises from 1 U/mL to 10 U/mL thrombin.

50. The method of any one of claims 45-49, wherein the fibrin cap comprises 2 U/mL of thrombin.

51. The method of any one of claims 45-50, wherein the fibrin cap comprises hyaluronic acid.

52. The method of any one of claims 45-51, wherein the fibrin cap comprises from 0.5 mg/mL to 1.5 mg/mL hyaluronic acid.

53. The method of any one of claims 42-52, wherein the backing component comprises a silicone dressing.

54. The method of any one of claims 30-53, wherein isolating the plurality of primary human melanocytes comprises enzymatic digestion.

55. The method of any one of claims 30-54, wherein isolating the plurality of primary human melanocytes comprises dissecting an epidermis of a skin sample from a subject from a dermis of the skin sample.

56. The method of any one of claims 30-54, wherein the plurality of primary human melanocytes are isolated without a mechanical dissection step.

57. A method of treating a skin condition of a subject in need thereof, comprising:
fabricating a patch device comprising: (i) a cellular component comprising a population of cells, the population of cells comprising a plurality of human melanocytes, (ii) a gel substrate having a first surface and a second surface, and comprising from 10 mg/mL to 15 mg/mL fibrin and 2 U/mL thrombin, and (iii) an adhesive applied to the second surface of the gel substrate; and
applying the second surface to a target tissue in a treatment area of a subject.

58. A method of treating a skin condition of a subject in need thereof, comprising:
fabricating a patch device comprising: (i) a gel substrate having a first surface and a second surface, and comprising from 10 mg/mL to 15 mg/mL fibrin and 2 U/mL thrombin, and (ii) a cellular component disposed within the gel substrate, the cellular component comprising a population of cells, wherein at least 80 percent of the population of cells is disposed within a distance from the second surface of no more than 50 percent of the height of the gel substrate; and
applying the second surface to a target tissue in a treatment area of a subject.

59. The method of claim 58, wherein at least 80 percent of the population of cells is disposed within a distance from the second surface of no more than 30 percent of the height of the gel substrate.

60. The method of claim 58, wherein at least 80 percent of the population of cells is disposed within a distance from the second surface of no more than 10 percent of the height of the gel substrate.

61. The method of any one of claims 57-60, further comprising debriding the treatment area before applying the second surface of the patch device to the target tissue.

62. The method of any one of claims 57-61, further comprising applying a pressure to the patch device oriented normal to the target tissue while the second surface of the patch device is applied to the target.

63. The method of claim 62, wherein the pressure is applied to the patch device for a time of less than 1 minute.

64. The method of claim 62, wherein the pressure is applied to the patch device for a time of from 1 minute to 72 hours.

65. The method of claim 64, wherein the pressure is applied to the patch device for a time of 24 hours to 48 hours.

66. The method of any one of claims 57-65, further comprising repeating the applying step.

67. The method of claim 66, wherein the applying step is repeated using a second patch device comprising (i) a cellular component comprising a plurality of human melanocytes, (ii) a gel substrate having a first surface and a second surface, and comprising from 10 mg/mL to 15 mg/mL fibrin and 2 U/mL thrombin, and (iii) an adhesive applied to the second surface of the gel substrate.

68. The method of any one of claims 57-67, wherein a backing component is coupled to the first surface of the gel substrate.

69. The method of claim 68, further comprising removing the backing component from the first surface of the patch device during the applying step.

70. The method of claim 69, further comprising removing the backing component from the first surface of the patch device after the applying step.

71. The method of any one of claims 57-70, wherein the subject has vitiligo, piebaldism or tinea versicolor or the target area includes scars or is a portion of a scarred area.

72. The method of any one of claims 57-71, comprising controlling the spatial distribution of cells within the patch device

73. The method of any one of claims 57-72, comprising delivering pigmented cells to the target area of the subject's skin.

74. The method of claim 73, comprising maintaining the spatial distribution of the pigmented cells within the patch device during application of the patch device to the target area.

75. The method of claim 73 or claim 74, comprising controlling the spatial distribution of the pigmented cells during transfer of the pigmented cells from the patch device to the target area.

76. A method of transferring pigment-producing cells to a target area of a surface of skin of a subject comprising:
delivering a patch device comprising the pigment-producing cells to the target area of the surface of the skin of the subject;
applying perpendicular pressure to the patch device in contact with the target area of the skin of the subject; and
transferring at least 80% of the pigment-producing cells from the patch device to the target area of the surface of the skin.

77. The method of claim 76, wherein applying the patch device is effective to repigment the target area of the surface of the skin to at least 90%, at least 95%, at least 97%, or at least 99% of a reference surface of the skin, as measured by reflectance spectroscopy or as determined by visual inspection.

78. A method of maintaining spatial distribution of pigment-producing cells on a target area of a surface of skin of a subject, the method comprising:
providing a patch device having a three-dimensional gel substrate comprising pigment-producing cells, the gel substrate having a spatial distribution of the cells of from 75,000 cells/cm² to 325,000 cells/cm² in an x-y plane of the gel substrate, the x-y plane is at most 500 micrometers thick;
applying the patch device to the target area of the surface of the skin; and
delivering the pigment-producing cells to the target area of the surface of the skin, the target area of the surface of the skin having a spatial distribution of the pigment-producing cells of 75,000 cells/cm² to 325,000 cells/cm².

79. A method of treating a subject with a skin depigmentation comprising administering to a target area of a surface of skin of the subject a patch device having a gel substrate comprising pigmented cells, wherein administration of the patch device is effective to repigment the target area of the surface of the skin to at least 80% of a reference surface of the skin, as measured by reflectance spectroscopy or as determined by visual inspection.

80. The method of claim 78 or claim 79, wherein the repigmentation of the target area is determined by measuring the melanin index of the target area.

81. The method of any one of claims 78-80, wherein the repigmentation of the reference surface is determined by measuring the melanin index of the reference surface.

82. The method of any one of claims 78-81, wherein administration of the patch device is effective to repigment the target area of the surface of the skin to at least 90%, at least 95%, at least 97%, or at least 99%.

83. A method of treating a subject with skin depigmentation comprising administering to a target area of skin of the subject a patch device having a gel substrate comprising pigment-producing cells, wherein administration of the patch device is effective to transfer the pigment-producing cells to the target surface of the skin more evenly than an alternative repigmentation treatment, as determined using reflectance spectroscopy.

84. The method of claim 83, wherein the alternative repigmentation treatment method comprises administration of cells in a non-viscous suspension, in a viscous suspension, using a rigid stamp, using a bandage, or using a tape.

85. The method of any one of claims 76-84, wherein the patch device is the cellularized patch device of claim 1.

86. The method of any one of claims 76-84, wherein the patch device is the cellularized patch device of claim 4.

87. The method of any one of claims 76-84, wherein the patch device is fabricated using the method of claim 30.

88. The method of any one of claims 85-87, wherein the patch device further comprises a backing component.

89. The method of claim 88, wherein the backing component is a silicone dressing.

90. The method of any one of claims 57-89, further comprising culturing at least a portion of the population of cells.

91. The method of any one of claims 57-89, further comprising culturing at least a portion of the population of cells for at least 5 passages.

92. The method of any one of claims 30-56, further comprising culturing at least a portion of the plurality of melanocytes.

93. The method of any one of claims 30-56, further comprising culturing at least a portion of the population of melanocytes for at least 5 passages.

94. The device of any one of claims 1-29, wherein at least a portion of the population of cells has been cultured.

95. The device of any one of claims 1-29, wherein at least a portion of the population of cells has been cultured for at least 5 passages.
